(19) United States
(12) Patent Application Publication
(10) Pub. No.: US 2026/0314999 A1
(43) Pub. Date: Oct. 15, 2026

(54) BRUXISM MANAGEMENT PLATFORM

(71) Applicant: ORAL SCIENCES, INC, Lewes, DE (US)

(72) Inventors: Lawrence Robins, Miami Beach, FL (US); Anthony Vocaturo, Manalapan, NJ (US); Mario Kern, Kramsach (AT)

(73) Assignee: ORAL SCIENCES, INC, Lewes, DE (US)

(21) Appl. No.: 19/643,520

(22) Filed: Apr. 9, 2026

Related U.S. Application Data
(60) Provisional application No. 63/785,952, filed on Apr. 9, 2025.

(51) Int. Cl. A61B 5/00; A61B 5/11; A61B 90/00; A61N 1/02; A61N 1/05; A61N 1/36

(52) U.S. Cl. CPC A61B 5/4836; A61B 5/1126; A61B 5/4557; A61B 5/682; A61B 5/7264; A61B 5/7282; A61B 5/742; A61B 5/746; A61B 90/06; A61N 1/025; A61N 1/0548; A61N 1/36003; A61B 2090/064; A61B 2560/0443; A61B 2560/0462

(57) ABSTRACT

Systems, methods, and computer-readable media for managing bruxism are provided, which may enable targeted, adaptable, and efficient feedback to patients and their caregivers. By combining dual-modality stimulation with an adaptive sensor-driven algorithm, such bruxism management systems may enable dynamic adjustment for providing clinical and user benefits that may include quicker muscle relaxation, enhanced comfort, and the potential for permanent reduction in bruxism episodes without relying on passive mouthguards. In some embodiments, a dual-modality, adaptive intraoral device may be provided for automated disruption and long-term retraining of bruxism.

### CROSS-REFERENCE TO RELATED APPLICATION(S)

This application claims the benefit of prior filed U.S. Provisional Patent Application No. 63/785,952, filed Apr. 9, 2025, which is hereby incorporated by reference herein in its entirety.

### COPYRIGHT NOTICE

At least a portion of the disclosure of this patent document contains material that is subject to copyright protection. The copyright owner has no objection to the facsimile reproduction by anyone of the patent document or patent disclosure as it appears in the Patent and Trademark Office patent file or records, but otherwise reserves all copyright rights whatsoever.

### TECHNICAL FIELD

This disclosure relates to the management of bruxism.

### BACKGROUND OF THE DISCLOSURE

Bruxism (e.g., a condition characterized by teeth grinding, gnashing, and/or clenching) has long haunted both adults and adolescents and may be associated with anxiety and mental health diagnoses, bite misalignment, neurological and health conditions, and even athletic and gym performance. It is painful and can cause multiple oral health problems, including, but not limited to, tooth damage, headaches, poor sleep quality, significant dental wear, temporomandibular joint (“TMJ”) disorders, and jaw pain. Yet, real-time treatment options are still impractical and short-term.

### SUMMARY OF THE DISCLOSURE

This document describes systems, methods, and computer-readable media for managing bruxism.

For example, a method for managing bruxism is provided as described herein.

As another example, a system for managing bruxism is provided as described herein.

As yet another example, a non-transitory computer-readable storage medium storing at least one program is provided, the at least one program including instructions, which, when executed by at least one processor of an electronic subsystem, cause the at least one processor to manage bruxism as described herein.

As yet another example, there is provided an intraoral neuromodulation system, including: (a) a tooth-supported or dental-arch supported intraoral body configured to be worn within an oral cavity of an individual user; (b) a sensing subsystem integrated within the intraoral body and including: at least one occlusal force sensing element configured to detect occlusal loading; and at least one inertial sensing element configured to detect motion parameters including at least one of acceleration or angular velocity; (c) a processing subsystem configured to: receive sensor signals from the sensing subsystem; and detect an occurrence of a defined oral event based on a combined evaluation of occlusal loading and motion parameters of the received sensor signals within a predefined temporal window, wherein the defined oral event includes a dynamically classified oral event based on multi-parameter sensor fusion; (d) an intervention subsystem configured to execute a device intervention in response to the detected occurrence of the defined oral event, the intervention subsystem including: a vibration output; and a neuromodulation stimulation subsystem output configured to alter neuromuscular activity without inducing a direct muscle contraction; (e) an escalation controller configured to select between a plurality of intervention stages for the device intervention based on a severity metric derived from at least occlusal force magnitude and motion intensity, wherein the plurality of intervention stages includes at least a first stage using the vibration output and a second stage using the neuromodulation stimulation subsystem output; and (f) a safety subsystem configured to: enforce stimulation output constraints using hardware-defined safety limits that are independent of the processing subsystem and not overrideable by firmware; and maintain a default-off stimulation state and require validation of multiple independent conditions prior to enabling stimulation, wherein: the hardware-defined safety limits include at least a hard current limit and a fault-triggered latched stimulation disable state, and the defined oral event is determined using a multi-parameter sensor fusion model.

As yet another example, there is provided an intraoral neuromodulation safety architecture, including: (a) a stimulation subsystem configured to generate charge-balanced stimulation pulses; (b) an isolation barrier separating the stimulation subsystem from a sensing and control subsystem; (c) a hardware safety enforcement subsystem including: (c1) a primary current limiting circuit; (c2) an independent secondary current limiting circuit; (c3) a DC-offset detection circuit configured to detect sustained electrode polarization; and (c4) a watchdog circuit configured to detect absence of a valid control signal; and (d) a fault latch configured to force the stimulation subsystem into a disabled state upon detection of any unsafe condition, wherein: stimulation defaults to an off-state upon power-up or reset; net delivered charge is limited; mean electrode potential remains within a predefined safe range; and stimulation remains disabled until a hardware-validated reset condition is satisfied.

As yet another example, there is provided a platform architecture for intraoral sensing and intervention including: an intraoral interface structure configured to engage teeth; a sensing subsystem; an event detection subsystem configured to detect events based on a combined evaluation of occlusal force and inertial motion parameters sensed by the sensing subsystem within a predefined temporal window; an intervention subsystem including at least two feedback modalities including mechanical feedback and neuromodulation; and a safety subsystem configured to enforce hardware-defined operational limits independent of software execution.

This Summary is provided to summarize some example embodiments, so as to provide a basic understanding of some aspects of the subject matter described in this document. Accordingly, it will be appreciated that the features described in this Summary are only examples and should not be construed to narrow the scope or spirit of the subject matter described herein in any way. Unless otherwise stated, features described in the context of one example may be combined or used with features described in the context of one or more other examples. Other features, aspects, and advantages of the subject matter described herein will become apparent from the following Detailed Description, Figures, and Claims.

### BRIEF DESCRIPTION OF THE DRAWINGS

The discussion below makes reference to the following drawings, in which like reference characters may refer to like parts throughout, and in which:

FIG. 1 is a schematic view of an illustrative system for managing bruxism, according to one or more embodiments of the disclosure;

FIG. 1A is a more detailed schematic view of a subsystem of the system of FIG. 1, according to one or more embodiments of the disclosure;

FIG. 1B is a more detailed schematic view of an illustrative example of an intraoral apparatus of the system of FIG. 1, in partial exploded view, according to one or more embodiments of the disclosure;

FIG. 1C illustrates an exemplary overall system architecture of an intraoral subsystem of the system of FIG. 1, according to one or more embodiments of the disclosure;

FIG. 2 is an occlusal view of an upper jaw of a user wearing the intraoral subsystem of FIG. 1C, according to one or more embodiments of the disclosure;

FIG. 3 illustrates an exemplary architecture for dual-domain isolation using the intraoral subsystem of FIG. 1C, according to one or more embodiments of the disclosure;

FIG. 4 illustrates an exemplary biphasic electrical stimulation path that may be provided by the intraoral subsystem of FIG. 1C, according to one or more embodiments of the disclosure;

FIG. 5 illustrates an exemplary process for staged escalation by the intraoral subsystem of FIG. 1C, according to one or more embodiments of the disclosure;

FIG. 6 illustrates a scenario for impact and contact-sport monitoring of an athlete using the intraoral subsystem of FIG. 1C, according to one or more embodiments of the disclosure;

FIG. 7 illustrates another exemplary architecture for dual-domain isolation using the intraoral subsystem of FIG. 1C, according to one or more embodiments of the disclosure;

FIG. 8 is a buccal view of an upper jaw of a user wearing the intraoral subsystem of FIG. 1C, according to one or more embodiments of the disclosure;

FIG. 9 illustrates yet another exemplary architecture for dual-domain isolation using the intraoral subsystem of FIG. 1C, according to one or more embodiments of the disclosure;

FIG. 10 illustrates a semi-exploded view of a modular electronics capsule system, according to one or more embodiments of the disclosure; and

FIG. 11 illustrates an exemplary process for manufacturing an intraoral device, according to one or more embodiments of the disclosure.

### DETAILED DESCRIPTION OF THE DISCLOSURE

The detailed description set forth below is intended as a description of various configurations of the subject technology and is not intended to represent the only configurations in which the subject technology can be practiced. The appended drawings are incorporated herein and constitute a part of the detailed description. The detailed description includes specific details for the purpose of providing a thorough understanding of the subject technology. However, the subject technology is not limited to the specific details set forth herein and can be practiced using one or more other implementations. In one or more implementations, structures and components may be shown in block diagram form in order to avoid obscuring the concepts of the subject technology.

Systems, methods, and computer-readable media may be provided to manage bruxism (e.g., any grinding, gnashing, or clenching of teeth, either subconsciously or due to some external force (e.g., athletic competition or high-impact event or accident). While some bruxism management systems may be configured to monitor bruxism using a single sensor (e.g., a force or electromyography (“EMG”) sensor) and then provide a single type of feedback (e.g., vibration or low-level electrical stimulation) to a user, these approaches can be limited in effectiveness and user comfort. For example, they may also fail to deliver precise, real-time disruptions that adapt to different clenching intensities over the course of a night. Some bruxism management systems (e.g., as may be disclosed by U.S. Patent Application Publication No. 20200330261-A1, which is hereby incorporated by reference in its entirety) may be configured to detect jaw-clenching episodes and deliver some form of feedback. While other bruxism management systems, such as certain novel bruxism management systems of this disclosure (e.g., dual-modality adaptive intraoral bruxism management systems), may be configured to provide an advanced, dual-modality feedback mechanism (e.g., by combining vibration and mild electrical muscle stimulation in a novel arrangement), an adaptive control algorithm, and an integrated sensor system that may be configured to cover multiple contact points within the mouth of a user (e.g., using force, pressure, audio, photoplethysmogram or photoplethysmography (“PPG”), galvanic skin response (“GSR”), acceleration, and/or EMG sensors). Such systems may enhance user comfort, customization, and long-term “retraining” of the neuromuscular pathways associated with bruxism. Such systems may be configured not only to provide diagnostic data and instantaneous communication with a patient's medical team, but also real-time relief and treatment as well. Such systems may provide a comfortable solution that can provide effective, long-term relief for those suffering from bruxism while empowering those individuals to achieve better sleep, improved oral health, and enhanced overall well-being.

A bruxism management system of the disclosure may include a bruxism treatment oral apparatus, which may be an overnight mouthguard and/or athletic mouthguard or other suitable protective mouthguard that may be designed to detect, analyze, and treat bruxism in real-time, such as a dual-modality, adaptive intraoral device for automated disruption and long-term retraining of bruxism. Such an intraoral device may feature any suitable sensor technology, such as advanced pressure sensors for precisely monitoring teeth clenching and grinding patterns, and/or biomedical sensors for tracking key physiological indicators to provide comprehensive medical feedback. By harnessing conductive polymer-based electrostimulation technology, such a bruxism management system of the disclosure may be configured to deliver targeted, non-invasive muscle stimulation to help relax the jaw, providing immediate relief, and/or reduces bruxism episodes over time. When integrated with Bluetooth or other suitable communication connectivity, a bruxism management system of the disclosure may enable an intraoral device to synchronize seamlessly with an artificial intelligence (“AI”)-powered patient application (“app”), which may offer users real-time insights while automatically transmitting instantaneous reports to any suitable remote entities (e.g., healthcare providers). This may ensure personalized treatment adjustments and proactive care, optimizing long-term oral health and sleep quality. Biofeedback, whether via EMG sensors, audio signals, and/or intraoral pressure sensors, can reduce the frequency and intensity of bruxism episodes, at least in the short term. Muscle retraining via disruptive stimuli is achievable. Repeated external stimuli (e.g., electrical, vibratory, and/or audio) can alter muscle activation patterns and reduce unwanted muscle contractions. Such approaches may rely on the principle of neuroplasticity, where the brain and neuromuscular system can adapt in response to new feedback loops and conditioning, even when the initial trigger for the movement is involuntary. Habit disruption and neuroplastic adaptation is possible, where muscle responses may be retrained using disruptive impulses and/or biofeedback (e.g., unconscious/conscious muscle retraining). Intraoral devices may enable more direct detection of bite force and/or enable more immediate localized feedback.

In some embodiments, a bruxism management system of the disclosure may include an intraoral device (e.g., a mouthguard) that may include, but is not limited to, an intraoral shell, overmold, pressure sensors, force sensors, biomedical feedback sensors, electrostimulation components, communication components, microcontrollers, power sources, and/or the like. An intraoral shell may be provided, which ought to be comfortable for a user to wear while sleeping (e.g., using a custom imprint and/or a 3D-printed or milled housing(s) with embedded compartment(s) (e.g., a ~2-5 mm thickness shell housing electronics in one or more overmolded and/or embedded compartments, which may be lightweight and minimizing bulk to avoid jaw fatigue or breathing interference when worn by the patient)), where the exterior may present a clean, minimal aesthetic, with clear and/or neutral colors that may provide a discreet, polished look. An overmold may be provided, (e.g., a medical grade overmold (e.g., Silicone) about the intraoral shell and/or its coupled component(s)). One or more pressure sensors and/or force sensors (e.g., piezoelectric sensor(s), thin-film pressure sensor(s), resistive pressure sensor(s), strain gauge sensor(s), surface electromyographic (“EMG”) sensor(s), and/or the like, whose calibration may be app-based or initially setup to set detection thresholds) may be integrated into or otherwise coupled to the shell (e.g., possibly in occlusal surfaces or near lower molars when provided by the shell worn by a patient). One or more biomedical feedback sensor(s) (e.g., infrared sensor(s) (e.g., for heart rate monitoring), temperature sensor(s) (e.g., for measuring body temperature (e.g., an LMT70 precision analog output temperature sensor)), accelerometer and/or gyroscope sensor(s) (e.g., to track movement, sleep stages, and/or activity levels), SpO2 sensor(s) (e.g., to monitor oxygen levels), bite sensor(s) (e.g., to measure bite force and/or asymmetry), etc.) may be integrated into or otherwise coupled to the shell. One or more electrostimulation component(s) or disruption mechanism(s) (e.g., conductive (e.g., polymer-based) electrostimulation component(s), piezo-electric stimulation component(s), and/or the like, such as mini electrodes based on conductive polymers located along the intraoral device (e.g., in the rear of the mouthpiece), haptic motor(s), e-stim module(s), and/or the like) may be integrated into or otherwise coupled to the shell (e.g., a vibration unit (e.g., a small haptic motor near the teeth or alveolar ridge when worn) and/or electrical stimulation (e.g., transcutaneous electrical nerve stimulation (“TENS”) and/or electronic muscle stimulation (“EMS”) pulses (e.g., mild or otherwise), adjustable amplitude, short bursts) with automatic step-up logic (e.g., each bruxing or bruxism event may be configured (e.g., via an app or firmware of the device) to trigger an initial pulse (e.g., if that fails, the device may increment intensity until grinding stops or it reaches a user-set max))). One or more communication component(s) (e.g., Bluetooth, Bluetooth Low Energy Module (“BLE”), and/or the like (e.g., by FDK or Toshiba)) may be integrated into or otherwise coupled to the shell. One or more low-power microcontroller(s) may be integrated into or otherwise coupled to the shell. One or more power sources (e.g., any suitable battery, such as a Li-Po or Li-ion battery, that may power the device throughout a use session (e.g., a night's sleep, an athletic endeavor, etc.) and that may be rechargeable and/or cordless and/or contactless (e.g., contactless rechargeable), but that may have no or few exposed parts for ensuring moisture protection and that may include protective circuitry for maintaining temperature thresholds and battery safety) may be integrated into or otherwise coupled to the shell.

Beyond such an intraoral device, a bruxism management system of the disclosure may also include any suitable additional components, including, but not limited to, a charging station, a bruxism management system app, a local user monitoring subsystem, and/or the like. A charging station may be provided (e.g., contactless charging station) for (re)charging a power supply of the intraoral device. A bruxism management system application (“app”) may be provided (e.g., a straight-forward, user-friendly app, such as one with easy slider controls and event-tracking dashboards), where the app may be configured to provide any suitable functionality, including, but not limited to, grinding episode tracking (e.g., to count events, duration, intensity used, etc.), stimulus efficacy (e.g., to log at which intensity level bruxism ceased, etc.), user alerts and trends (e.g., to securely transmit data to the patient via the app for daily/weekly insights), and/or the like. A local user monitoring subsystem (e.g., a user's laptop computer, smart telephone, etc.) may be configured to store and run any suitable bruxism management system application and that may be configured to store, process, and communicate any suitable data/instructions/commands with the intraoral device and/or any remote subsystems (e.g., a server of a user's healthcare provider) via any suitable communication network (e.g., cellular network, etc.) for monitoring the intraoral apparatus worn by a patient. Great care may be taken to ensure (1) miniaturization (e.g., fit sensor arrays, battery, stimulation modules, and/or haptic motor(s) in a small device (e.g., in the intraoral device (e.g., within its overmold))), (2) saliva/waterproofing (e.g., to ensure durability and safety of the intraoral device in a wet environment), (3) user variability (e.g., to ensure different bite forces, mouth sizes, sensitivity levels may be accommodated), and (4) integration (e.g., to ensure coordination of electronics design, mouthguard manufacturing, and app software).

A bruxism management system of the disclosure may be configured to utilize such an intraoral device worn by a patient to take an initial assessment by capturing and processing baseline and vital signs upon initial placement for each use. Data may be transmitted to the app for storage. The app may also be communicatively coupled to the patient's medical providers to whom data may be automatically transmitted. The device in conjunction with the app may be configured to detect jaw clenching and teeth grinding and transmit the data, which may be configured to trigger another reading and transmission of biomedical data. The completion of such a process may trigger any suitable electro-stimulation by any suitable electrostimulation component(s) of the system. Biomedical data may be transmitted either throughout or periodically during the treatment. The beginning and ending of a treatment may also be transmitted. A final biomedical reading may also be transmitted. This cycle may repeat as needed throughout the night or other time frame during which the intraoral device may be worn by the patient. A level of stimulation and/or any other suitable characteristics of operation may be adjustable (e.g., mechanically or through the app). In some embodiments, the app may be configured to automatically run a treatment by starting milder and working its way up automatically based on pre-set boundaries in the app. Therefore, this may provide a next-generation, app-integrated mouthguard designed to detect and disrupt bruxism through adaptive vibration and/or electrical impulses. Auto-escalation logic may be configured to ensure clenching is stopped without waking the user, while the companion app may log and auto-transmit medical data and events and allow fine-tuning. Therefore, the system may include an intraoral wearable device (e.g., a mouthguard form factor) that may be configured to detect and disrupt bruxism episodes in real time. A disruption level (e.g., vibration and/or electrical stimulus) may be user-adjustable via a companion app. Data may be collected on grinding frequency/duration as well as other medical symptoms, and intensity of feedback may be configured to auto-escalate if bruxism persists, thereby ensuring effective interruption without fully waking the user. Initial setup and programming of the system may be accomplished in any suitable manner. In some embodiments, before first use by a patient, the patient or their caregiver may use the app to configure baseline parameters, such as stimulation intensity, vibration levels, and sensitivity thresholds. This setup may include determining the patient's comfort and pain thresholds, so the system may determine how strongly to disrupt clenching without causing excessive discomfort using the intraoral device. During use by a patient (e.g., during sleep, during an athletic event, etc.), an automatic “combo therapy” configuration may be used. For example, once such baseline parameters are set, the system may configure the intraoral device to run an automatic program (e.g., locally on the intraoral device or using instructions streamed to the intraoral device from the local user monitoring subsystem). Such a program (e.g., running app) may be configured to gradually increase or decrease the electrical and vibrational stimulation based on the patient's pre-defined tolerance levels and detected bruxism intensity (e.g., grinding force, frequency, etc.). If the patient does not manually adjust settings during the night, the app may be configured to follow this predetermined schedule, subtly ramping up or down stimulation as needed. Manual adjustments (e.g., real-time override) may be enabled by the system. For example, if the patient wakes up or experiences discomfort, they can open the app (e.g., using the local user monitoring subsystem, which may provide an intuitive user interface, or using a small user controller that may be built into the intraoral device) to “dial up” or “dial down” the intensity or length of the disruptive pulses. Such manual control may be configured to allow on-the-fly tweaks if the automatic therapy feels too strong, too weak, or is disturbing the patient's sleep. The system may be configured to enable any suitable data sharing and ongoing tuning. For example, the app may be configured to log usage and grinding data, which the patient can share with their dentist. Together, they can fine-tune the stimulation patterns, intensities, or ramp schedules for optimal long-term results.

A bruxism management system of the disclosure may be configured to provide dual-modality disruption, such as simultaneous vibration and electrical stimulation (e.g., combination therapy). However, there may be some situations that may use vibration only, and some situations that may use stimulation only, and some other situations that use both stimulation and vibration at the same time or in a quick alternating fashion according to any suitable pattern or program. Therefore, a system may be configured to provide a dual treatment with the ability to consider one or both treatments depending on the patient's needs and if they already have TMJ and/or other conditions that may require one or the other or both treatments simultaneously, as different patients may have different needs. Unlike a single feedback approach, some bruxism management systems of the disclosure may be configured to deliver two or more distinct forms of feedback, such as (1) a mechanical vibration to the patient (e.g., at or near the teeth of the patient) and (2) a targeted electrical pulse (e.g., EMS and/or TENS-like pulses) to the patient (e.g., directed at the masseter muscle insertion of the patient). This combination may more effectively interrupt ongoing bruxism episodes without fully awakening the user. There may be differences in the nature of the stimulus (e.g., mechanical vibration versus electrical pulse). For example, mechanical vibration may use a small motor or similar mechanism to create physical vibrations that may travel through the teeth or a portion of the mouthguard. Such feedback may be configured to feel more like a subtle buzzing or shaking sensation (e.g., an external, mechanical force) and/or may be configured to primarily alert the user by jostling the jaw or teeth, thereby creating an immediate tactile interruption of clenching. Alternatively, electrical pulse may be configured to use electrodes that may deliver mild electrical currents (e.g., TENS/EMS waveforms) to the jaw muscles or the nerves controlling them. This can directly affect muscle fibers by causing a brief contraction or relaxation, thereby neuromuscularly interrupting the bruxism event. Compared to vibration, electrical stimulation may be configured to engage the muscle more directly, often with less overall movement but with a distinct “tingling” or localized pulse feeling. There may be any suitable differences in positioning for vibration verses electrical pulse feedback targeting within the mouth. Vibration feedback may be targeted towards or applied to or placed on or near the occlusal (e.g., biting) surface of the teeth, or at a buccal extension close to the molars. The goal may be to mechanically disrupt the clenching by vibrating the teeth themselves or the portion of the mouthguard directly in contact with them. This approach may leverage the bone conduction of the jaw/teeth for an immediate jarring effect. Electrical pulse feedback may be targeted towards or applied to or positioned on the cheek-facing (e.g., buccal) side of the mouthguard, near the masseter muscle insertion or other jaw muscles implicated in bruxism. Electrodes may be configured to contact the inner cheek/gum tissue so they can transmit electrical impulses directly to the underlying muscle or nerve pathways. This location may focus on modulating muscle activity from within the oral environment, rather than vibrating the teeth. Therefore, vibration may be a mechanical disruption felt as a buzz or shake, whereas an electrical pulse may be a neuromuscular disruption that may more directly affect muscle firing. The vibration component may typically target the teeth or mouthguard surface for a broad jolt, while the electrical component may typically target the muscles via electrodes near the cheek or jaw insertion points, delivering localized electrical stimulation. In some embodiments, the vibration sensor and motor may be typically placed near the occlusal surfaces (e.g., biting area) of the mouthguard, especially around the molars or premolars where bruxism forces may be greatest. A small pressure or force sensor may be configured to detect when the patient clenches or grinds, and a compact vibration motor (e.g., a motor or other suitable feedback component embedded in the same area of the guard) may be configured to transmit a mild mechanical “jolt” through the teeth. This localized vibration may be configured to alert the patient's neuromuscular system and help interrupt ongoing clenching without directly contacting soft tissue. Electrical stimulation (e.g., TENS and/or EMS) may be configured to provide mild electrical pulses, on the other hand, which may come from electrodes positioned along the buccal (e.g., cheek-facing) side of the guard, near the lower molars, or in any other suitable position(s). These electrodes may be configured to rest against the inner cheek or gum area, allowing the current to pass into the nearby masseter muscle when the intraoral device is worn by the patient. By delivering brief TENS or EMS impulses, the device may specifically disrupt the masseter's contraction pattern, thereby reducing the force of clenching and helping to break the habit over time.

A bruxism management system of the disclosure may be configured to utilize a real-time adaptive control algorithm (e.g., of a system app) that may automatically adjust the intensity and/or type of feedback based on the detected severity of clenching and/or any other suitable detected characteristics of the patient. For example, if a patient begins mild clenching, only a gentle vibration may be triggered by the system, whereby if a strong clench occurs, the electrical stimulation of the system may be automatically scaled up, ensuring an immediate but tolerable disruption. In some embodiments, the algorithm may be configured also to factor in user-specific parameters (e.g., jaw size, typical clench force, etc.), which may be learned over time, thereby providing a non-static threshold system.

A bruxism management system of the disclosure may be configured to include a multi-point sensor array, such as a plurality of sensors (e.g., pressure sensors, strain gauges, integrated electromyography (“EMG”) electrodes, and/or the like) that may be embedded at strategic positions on or near the occlusal surfaces and along the buccal extension of a custom mouthguard. This multi-sensor approach may offer finer resolution of clenching activity, thereby enabling the device to detect not only overall force but also the location and/or the pattern of clench events. This may differ from a simpler single-sensor or external detection sensor system, thereby providing improved accuracy and fewer false positives.

A bruxism management system of the disclosure may be configured to include a custom-fitted, intraoral appliance, such as a custom dental-fitted mouthguard that may be configured to house or at least provide structural form for the electronics in a sealed, biocompatible casing. In some embodiments, masseter-targeting electrodes may be positioned on the mouthguard in any suitable manner so as to be proximate the buccal side near the molars when worn by a patient, such as for delivering electrical impulses directly into the muscle region and/or at the jawbone insertion point. One or more vibration motor(s) may be placed in any suitable manner so as to be near the occlusal surfaces to jolt the teeth gently when the mouthguard is worn by the patient. This specialized, fully intraoral arrangement may contrast with systems that may use either an external electrode on the cheek or a single sensor in the mouth.

A bruxism management system of the disclosure may be configured to enable long-term retraining and data logging. For example, an intraoral device may incorporate an onboard app with memory for local logging and/or a wireless data logging feature for communicating with a log on a local monitoring subsystem. In some embodiments, if there is a loss of a wireless connection, the system may be configured to store data in memory of the intraoral device until it reconnects with a monitoring subsystem, such that if the app of a monitoring subsystem is communicatively decoupled or out of reach to connect and serve real time data to the intraoral device live, a buffer on the intraoral device may be used to store some user data until it reconnects to the monitoring subsystem app. Also, this may allow the device to not need the monitoring subsystem app to run once the program is set and therefore the device may not require an active communication link with the monitoring subsystem to activate and carry out a bruxism management program on the intraoral device. Over successive uses (e.g., nights), such a system may be configured to track bruxism frequency, intensity, and response to feedback. By systematically interrupting clenching events in real time, the system may enable patients to experience progressive neuromuscular “relearning,” thereby eventually reducing the overall incidence of bruxism. This collected data can also be reviewed by dentists or clinicians to guide further treatment.

A bruxism management system of the disclosure may be configured to enable user-adjustable profiles. Through a companion system app running on a communicatively coupled processing subsystem (e.g., a user tablet or laptop or smartphone) or a small interface onboard the intraoral device, users or clinicians can be enabled to customize feedback thresholds, maximum stimulus levels, or vibration frequencies. This level of personalization may be especially critical for individuals with highly variable bruxism patterns or heightened sensitivity to certain stimuli.

A bruxism management system of the disclosure may be configured to include any suitable battery and power management capabilities. For example, the intraoral device (e.g., mouthguard) may be powered by a thin, rechargeable battery module that may be integrated into or otherwise coupled to a posterior portion of the device. Wireless charging contacts and/or a sealed micro-USB port may be provided by the battery module. The system may be designed for overnight use, featuring low-power, smart wake-sleep cycles that may be configured to conserve battery power and only fully activate upon detecting clenching. Some power savings may be software related, whereby the definition of power savings may involve running minimally or non-communicatively with the local monitoring subsystem until the intraoral device's pressure sensors or other suitable sensors are depressed or otherwise triggered by a user, which may then trigger wireless data communication to the app of the local monitoring subsystem to determine if the intraoral device ought to turn on any suitable component(s) (e.g., feedback component(s), thereby using more power) or if it ought to just continue transmitting data to the monitor subsystem without ramping up any additional components of the intraoral device (e.g., a partial sleep mode). Alternatively, this may be done entirely onboard the intraoral device without any communication with an app on a local monitoring subsystem. In some embodiments, an intraoral device may be configured to support continuous BLE connectivity for real-time communication, allowing it to transmit sensor data and receive updates instantly throughout the night from the local monitoring subsystem running a system app. This approach may facilitate immediate user input (e.g., changing vibration intensity) and instantaneous logging of bruxism events with the more user friendly and processing robust local monitoring subsystem. BLE may be relatively power-efficient, maintaining an always-on link but can still increase overall battery consumption, especially for extended nighttime usage. In some embodiments, the system may be configured to use an on-demand (e.g., event-driven) approach that can keep the BLE module in a low-power or sleep mode until a specific trigger occurs, such as the user pressing a sensor, reaching a threshold clench force, or manually activating a button. Only then may the device wake, establish a brief connection, exchange necessary data or commands with the app of the local monitoring subsystem, and return to sleep. This may significantly conserve battery life but may introduce a short delay during wake-up and reconnection. BLE chips may be configured to also offer hybrid strategies, such as running minimal background communication but sleeping deeply between intervals. The methodology to be used may depend on whether constant data logging and instant responsiveness outweigh the importance of maximizing battery longevity. Alternatively, connectivity with a local monitoring subsystem may not be necessary when the intraoral device is in use within the patient, but may instead be running its own local program app and data logging, which can be shared with and/or updated by an app running on a monitoring subsystem during intraoral device recharging or other periods of time when not using its battery for in-mouth patient treatment.

In particular embodiments, a bruxism management system of the disclosure may be configured to include a custom-fitted upper mouth guard and/or a custom-fitted lower mouthguard that may be fabricated from medical-grade thermoplastic. Some bruxism-focused treatments may target the lower jaw, where the intraoral device may include only or at least a lower mouthguard for one or more reasons. For example, the masseter muscle inserts along the ramus of the mandible, close to the lower molars. Delivering electrical pulses or vibration near that insertion point may directly impact the muscle's contraction patterns. Sensors measuring bite force or jaw clenching (e.g., pressure, strain gauges) may preferably sit in the occlusal surface (biting area) of a lower mouthguard. Electrodes for TENS/EMS can more readily contact the buccal (cheek) side of the lower molars or gingiva, offering a shorter path to the masseter's motor points. A single lower mouthpiece is typically simpler and more comfortable than a full upper-lower setup. Users can more easily adapt to one device, and design complexity (electronics, power) is lower with a single device setup. However there may be reasons to provide an intraoral device with only or at least an upper mouthguard. For example, dual-jaw contact may be useful. Some designs may be configured to measure or disrupt clenching between both arches, ensuring that whichever teeth are in contact (upper vs. lower) still trigger feedback. This may yield more comprehensive detection of grinding patterns if someone's bite alignment or jaw movement is unusual. Vibrations introduced near the upper molars can propagate differently through the craniofacial structure. If it is useful to jolt the entire jaw, some systems may include an intraoral device with both upper and lower mouthguard portions, such that both arches may receive mechanical disruption. A dual-arch design with sensors/electrodes on both the upper and lower mouthpieces may offer the highest detection accuracy but may be at the cost of more bulk, complexity, and user adjustment. A lower mouthguard intraoral device may be one potential solution for placing sensor(s) in the occlusal area to detect clenching, with electrode(s) and/or vibration module(s) near the masseter insertion on the buccal side, so as to provide most comfort for a user and direct access to the primary muscle responsible for bruxism. An upper mouthguard may be included in certain embodiments, such as if symmetrical detection is useful and/or if the user's clenching pattern is heavily biased toward the upper teeth. A lower-jaw-centric design may be generally sufficient and more straightforward for achieving effective masseter disruption. If other iterations require more robust detection or a broader contact area, the system may add or alternatively use upper-arch elements for additional feedback or sensor coverage. Embedded within the occlusal plane of the mouthguard may be force-sensitive resistors that may be arranged in a grid pattern to detect localized contact pressures. A small vibration motor may be placed near the rear molars when the mouthguard is worn to ensure a mechanical jolt can be transmitted through the teeth when activated. On the buccal extension (e.g., cheek-facing side) of a mouthguard, two or any other suitable number of flexible electrodes may be configured to contact the mucosa near the jaw's angle when the mouthguard is worn. These electrodes may be configured to deliver a mild electrical pulse (e.g., TENS or EMS parameters) set to a short, rapid burst that may be configured to interrupt the masseter contraction reflex of the patient. The amplitude, waveform, and/or pulse duration can each be adjusted digitally by the microcontroller. An onboard adaptive algorithm may be configured to continuously monitor incoming sensor data (e.g., data from one or more sensors of the mouthguard). A microcontroller and its adaptive algorithm may be configured to primarily reside onboard the intraoral device itself, rather than relying entirely on an external smartphone or tablet app. The mouthguard may be configured to house all the critical electronics (e.g., pressure sensors, electrodes, a small battery or power source, and a compact microcontroller chip). By including the hardware and core logic within the mouthpiece, the system can monitor bruxism events (e.g., clenching force, muscle activity, etc.) and respond instantly with electrical or vibrational feedback. This real-time reaction may be useful as even a brief delay waiting for local monitoring subsystem (e.g., phone-based) processing could undermine the device's ability to disrupt a clench before it causes damage or discomfort.

Meanwhile, an app running on a local monitoring subsystem (e.g., a smartphone app) may be used for configuring and guiding the mouthguard's settings. Users can adjust or fine-tune parameters, such as maximum stimulation intensity, ramp-up schedules, and/or sensor sensitivity thresholds using the app interface of a more user friendly local monitoring subsystem than any user interface that may be provided by the intraoral device itself. Such an app may be configured to send such preferences to the mouthguard's onboard microcontroller, thereby ensuring that, once in place, the device knows precisely how to respond under different clenching scenarios. Critically, if the local monitoring subsystem (e.g., smartphone) is out of reach or its battery is drained, the adaptive logic may still be configured to run independently on the mouthguard, since the essential detection and feedback loops live on the device itself. This design approach may blend convenience (e.g., via local monitoring subsystem app-based customization) with the reliability of an autonomous, microcontroller-driven system that can instantly detect and interrupt bruxism without continuous external input. When a threshold pressure or EMG signal indicates a clench event, the device may be configured to first initiate low-intensity vibration. If the force surpasses a second threshold or persists beyond a defined duration, the device may be configured to deliver an electrical pulse. This tiered approach may ensure minimal disturbance for mild grinding while reliably disrupting stronger clenching episodes. In some embodiments, stimulation and vibration may each be broken up on a scale of 1 to 10 or any other suitable range with preset incremental increases in time and or stimulation/disruption vibration. For example, the device may have a disruption vibration (e.g., if user activated in set up) 1 to 10, and electro stimulation (e.g., if user activated in set up) also 1 to 10. In terms of its pattern, there may be many options, such as a ramp up during the program or a straight consistency or even a zigzag pattern. Patterns of how stimulus delivered over time ramp up consistent or inconsistent delivery during patient data testing may be determined and used (e.g., via one or more trained models or otherwise). Time duration may be chosen by the depression of the device. So long as the device is being depressed, the program may run. So the time portion of the device may be considered only during the time of depression of the device, although other embodiments are possible. Over several weeks, consistent disruption of clenching episodes may condition the user's neuromuscular system to avoid bruxism. Any suitable data (e.g., nightly clench counts, average force, the number of feedback events, and/or the like) may be stored in onboard memory or transmitted wirelessly to a companion mobile app. Some such data that may be collected is how many times the patient is grinding and for its duration, and the result of serving various stimulus indicated above being vibration and electric stimulus having a lot to do with what the patient and physician has decided during the setup of the unit. There may be several other features and medical data that can be collected on the unit. Various types of data may be collected and processed using any suitable sensors for any suitable purpose, including determining feedback types and/or schedules. For example, any suitable SpO2 sensor may be provided to monitor oxygen levels. Any suitable bite sensor may be provided to measures bite force and asymmetry. Any suitable heart rate and oxygen saturation and/or (“PPG”) infrared sensor(s) may be provided (e.g., to track pulse via LED and photodiode, providing beats-per-minute (“BPM”) and blood oxygen (“SpO₂”), which may enable heart rate variability (“HRV”) analysis if data sampling is frequent enough). Blood pressure and/or pulse wave velocity (“PWV”) may be sensed, where estimations may be derived from arterial pulse transit times, while this may rely on stable optical/electrical pathways in the mouth. Salivary analysis (e.g., biomarker sensors), which may detect stress hormones (e.g., cortisol), glucose (e.g., for metabolic health), and/or saliva pH, where electrochemical and/or microfluidic chips may sample saliva in real time. Temperature monitoring (e.g., LMT70), which may include thermistors and/or IR sensors that may capture oral mucosal temperature, which may be useful for fever detection or circadian rhythm insights. Pressure, strain, and/or motion sensors (e.g., force-sensitive resistors (“FSRs”), accelerometers, and/or strain gauges) may be provided that may measure bite force and/or jaw movements, which may be core to bruxism detection and real-time feedback triggers. ECG/EMG-like electrical measurements may be captured, where electrodes can record muscle signals (e.g., EMG for clenching) or limited cardiac signals (e.g., ECG), and where oral ECG may be feasible with specialized electrodes. Respiration rate may be detected, which may be derived from subtle changes in PPG waveforms or accelerometer data, and which may indicate breathing patterns during sleep or while at rest. Sleep stage approximation (e.g., accelerometer +heart rate data can hint at sleep depth (e.g., light, deep, or REM), which may require stable, low-noise signal capture overnight). Activity tracking and/or calorie estimates may be detected (e.g., motion sensors may detect overall movement and can estimate energy expenditure, which may be more relevant for day-wear, but still feasible if the device is worn outside of sleep). VO2 max and/or fitness indicators may be detected (e.g., advanced analysis of heart rate plus activity data to gauge aerobic fitness, which may rely on sustained monitoring and more robust algorithms). Stress and recovery indicators may be detected, which may combine HRV, sleep quality, and possibly salivary biomarkers for a daily “stress score” or readiness metric, which may provide holistic insight into autonomic balance and recovery. Accelerometer and/or gyroscope sensors may be provided, which may track movement, sleep stages, and activity levels. Notifications and alerts may be generated (e.g., real-time triggers for anomalies (e.g., irregular heartbeat, oxygen drops, excessive clenching), which may transmit data to a companion app, dentist's dashboard, and/or direct user alerts). By integrating such sensor modules (e.g., PPG, accelerometers, strain gauges, chemical sensors, etc.) into a mouthguard form factor, a wide range of biofeedback data may be gathered beyond just bruxism detection. Clinicians and/or the app (e.g., automatically through app programming) can review this data to help fine-tune settings or track progress. In some particular embodiments, a bruxism management system of the disclosure may be configured to include an intraoral device (e.g., mouthguard) that may additionally or alternatively include one or more different sensor types (e.g., accelerometers to detect subtle jaw movements), a specialized algorithm that may be configured to modify stimulation frequency throughout the night if the user becomes desensitized to initial intensities, and/or the like. Such bruxism management system of the disclosure may be configured to provide (1) dual-modality feedback (e.g., vibration and mild EMS/TENS) employed simultaneously inside the mouth, (2) an adaptive, tiered threshold system for real-time customization of feedback intensity, (3) a multi-sensor array for more precise detection and reduced false triggers, and (4) a fully integrated, custom-fitted design with advanced data logging and user-adjustable profiles. Such an intraoral bruxism management system may provide more targeted, adaptable, and efficient feedback than single-mode devices. By combining dual-modality stimulation with an adaptive sensor-driven algorithm, intraoral bruxism management systems of this disclosure address limitations of other solutions, such as those that may rely on a single form of feedback or that may lack dynamic adjustment. Clinical and user benefits may include quicker muscle relaxation, enhanced comfort, and the potential for permanent reduction in bruxism episodes without relying on passive mouthguards alone.

The following is a description of various parts of a bruxism management system according to various embodiments of the disclosure, which may be configured as a mouthguard-style device with real-time biosensing, vibration/EMS disruption, wireless charging, and app integration, and which may include, but is not limited to, the following: (1) a microcontroller/system-on-chip (“SoC”), (2) biofeedback/biosensor module(s), (3) vibratory disruptor/motor module(s), (4) EMS/TENS driver module(s) (e.g., electrical stimulation), (5) rechargeable battery (e.g., ultra-thin Li-ion or Li-Po battery), (6) wireless charging module/coil, (7) printed circuit board (“PCB”) and interconnect, (8) encapsulation/enclosure material(s), (9) firmware/app integration, and/or the like. A microcontroller and/or SoC may be configured to coordinate sensor readings, drive the vibration/EMS outputs, manage power, and/or handle wireless communication from the intraoral device to an off-device app (e.g., a smartphone app) using any suitable communication protocol (e.g., via Bluetooth Low Energy), whereby such a component may provide ultra-low power consumption, integrated BLE for app communication, and/or enough I/O pins for any suitable sensors, EMS drivers, and/or any debugging (e.g., a Nordic Semiconductor nRF52 Series, an Espressif ESP32 (e.g., ESP32-C3 if RISC-V to be used), Dialog Semiconductor/Renesas DA14531, and/or the like). Biofeedback/biosensor module(s) may be configured to detect any suitable biometric data, such as muscle activity (e.g., EMG), heart rate, and/or other signals, and may be used to monitor clenching or user stress levels (e.g., EMG sensor(s) for muscle tension (if focusing on jaw clench detection) (e.g., Advancer Technologies' MyoWare modules may be adapted into a smaller form factor), accelerometer/pressure sensor(s) to detect bite force or jaw movement (e.g., Bosch Sensortec (e.g., BMA series) or TE Connectivity pressure sensors, or the like), pressure sensor(s) (e.g., piezoelectric sensors that may generate an electrical charge (for measurement) in response to pressure, which can be very sensitive to changes in pressure and may be useful for detecting the dynamic forces during teeth grinding, where piezoelectric materials like lead zirconate titanate (“PZT”) can be embedded in a mouthguard to measure the intensity and frequency of pressure during bruxism), resistive pressure sensor(s) that may be configured to change resistance based on the applied pressure, thin film pressure sensor(s) may be configured to measure the amount of force or pressure exerted on specific points of the mouthguard, and/or the like, where the mouthguard can be embedded with small force sensors to detect when excessive pressure is applied, which can be a clear sign of bruxism, and they can detect the location of the pressure, biomed sensor(s) (e.g., infrared sensor(s) (e.g., to monitor heart rate)), temperature sensor(s), accelerometer and gyroscope sensor(s) (e.g., to track movement, sleep stages, and activity levels to help in determining the wearer's physical activity, sleep quality, and overall movement), SpO2 sensor(s) to measure blood oxygen levels for providing insights into respiratory health, sleep quality, and potential sleep apnea, strain gauge sensor(s) to sense the pressure exerted on the teeth and gums for identifying abnormal forces, surface electromyographic sensor(s) to measure the electrical activity produced by muscle contractions for helping identify episodes of bruxism and clenching, bite sensor(s) to measures bite force and asymmetry, and/or the like. Vibratory disruptor/motor module(s) may be configured to provides a mild vibration to the mouthguard when bruxism is detected, thereby “jolting” the user's neuromuscular pattern (e.g., a coin or pancake vibration motor (e.g., with a small form factor (e.g., 4-8 mm thick), low operating voltage (e.g., 3 V), and minimal current draw). EMS/TENS driver module(s) may be configured to generate low-level electrical pulses to disrupt muscle contractions (e.g., may enable an adjustable pulse width/frequency/amplitude, may handle safe isolation from user contact, and may be powered by low DC voltage (e.g., 3.7 V nominal from Li-ion)) and/or may include TENS alternatives, such as a conductive polymer-based electrostimulation that may involve using electrically conductive polymers to deliver electrical signals for therapeutic or diagnostic purposes, offering a flexible and biocompatible platform for applications like neural tissue engineering and bioelectronic devices and/or may include piezoelectric stimulation that may be used for muscle relaxation by delivering low-intensity electrical signals to muscle fibers, promoting blood circulation, reducing muscle tension, and aiding recovery (e.g., a small piezoelectric stimulation systems may be thin, flexible, and wireless devices designed for wearable applications that may generate stimulation through body movements, external pressure, and/or tiny power sources). A rechargeable battery may be configured to power the microcontroller(s), sensor(s), and/or motor(s)/stimulation driver(s), and ought to be safe in a moist environment (e.g., thorough sealing), with any suitable capacity (e.g., 20-100 mAh), and any suitable ultra-thin profile (e.g., 3-4 mm or less), where a specific LiPo rechargeable battery may be about 10 mm×10 mm with a voltage of about 3.7 V and holding a charge up to 24 hours. A wireless charging module/coil may be configured to allow a user to place the mouthguard on a charging pad (e.g., a Qi-based or a custom inductive solution), which may eliminate external connectors, which may be important for waterproofing. A printed circuit board (“PCB”) and interconnect may be configured to house, support, and/or electrically couple the microcontroller(s), sensor(s), driver(s), battery coupling(s), and coil alignment if integrated (e.g., a flexible or rigid-flex PCB to conform to mouthguard shape, biocompatible coatings or encapsulation to protect electronics from saliva, etc.). Encapsulation / enclosure material(s) may be configured to protect electronics from moisture, ensure comfort in the mouth, compliance with medical safety standards (e.g., FDA Class II device), and/or the like (e.g., medical-grade silicone (e.g., soft, used in mouthpieces), thermoplastic polyurethane (“TPU”) for flexible enclosures, acrylic or polypropylene (e.g., for more rigid sections, if needed), and/or the like). Firmware/app integration may be configured such that firmware may run on the microcontroller to handle sensor data, disruptor actuation, and/or communication, while the app may be configured to set user preferences, log data, provide analytics, and/or the like (e.g., Nordic SDK (e.g., if using an nRF52 chip), Arduino/PlatformIO for easy prototyping, native iOS/Android app with BLE libraries to read data from the device, cloud or local data logging, etc.). The circuit board (e.g., PCB) and electronics may be provided by combining a microcontroller and wireless charging receiver and small battery and sensor(s) and motor driver(s) on a rigid-flex board, while any suitable encapsulation may be used to seal the electronics in a medical-grade silicone mold shaped like a mouthguard or embedded sub-section for the PCB(s), while a Qi or custom inductive charging station may be provided as an external wireless charger to rest the mouthguard on, while firmware may be configured to manage real-time sensing (e.g., EMG or pressure sensor sensing) and trigger any suitable vibratory or EMS disruptor(s) and enable communications via BLE with a system mobile app, which may be configured to let any suitable user(s) track usage, set intensity levels, learn how often a patient clenched, and/or the like.

Therefore, a bruxism management system according to various embodiments of the disclosure, which may be configured to carry out any suitable detection, disruption and biofeedback, mobile app integration, and comfort and wearability. Detection may provide real-time sensing (e.g., detect teeth clenching early (e.g., via force/pressure sensors, accelerometers, and/or EMG signals)) and use an adaptive threshold (e.g., user calibration for bite force to differentiate normal contact from actual bruxing). Any suitable disruption and biofeedback may provide multi-mode stimulus (e.g., vibration and/or mild electrical pulses (e.g., TENS/EMS-like)) and intensity control (e.g., automatic step-up (e.g., if the initial stimulus does not halt grinding, the device may be configured to automatically escalate intensity incrementally until it does) and/or user-defined limits (e.g., users may be enabled to set a maximum intensity in the app to avoid discomfort or full wakefulness)). Mobile app integration may provide adjustable settings (e.g., users can fine-tune intensity for vibration/e-stim via a smartphone app, ensuring the lowest effective level that still keeps them asleep), data logging (e.g., grinding frequency and duration to count how many bruxism events occur, stimulus effectiveness to log how many times stimulus was escalated before grinding stopped, and trending and reporting to display or otherwise present nightly/weekly stats (e.g., “you had 12 clenching episodes; 2 required higher intensity”), user feedback loop (e.g., if the system escalated intensity often, the user may realize they need to dial it back down or alter thresholds, which may encourage iterative user/clinical calibration for optimal results). Comfort and wearability may provide an ergonomic mouthguard (e.g., slim, snug-fitting, medical-grade materials (e.g., silicone/acrylic), etc.), with minimal disruption (e.g., gentle pulses that may break the clench reflex but do not fully awaken the user), and hygiene and durability (e.g., waterproof/water-resistant enclosure(s) for saliva exposure and easy cleaning). A bruxism management system according to various embodiments of the disclosure may be configured to take an initial assessment by processing baseline and vital signs upon initial placement for each use. Data may be transmitted to the app for storage. The app may also be communicatively coupled to the patient's medical provider(s) to whom data may be automatically transmitted. The system may be configured to detect jaw clenching and teeth grinding and transmit the data, which may trigger another reading and transmission of biomedical data. The completion of that process may trigger the electro-stimulation device.

Biomedical data may be transmitted either throughout or periodically during the treatment. The beginning and ending of the treatment may also be transmitted. A final biomedical reading may be transmitted. This cycle may repeat as needed throughout the night. A level of stimulation may be adjustable through the app. It may be configured to automatically start milder and work its way up automatically based on pre-set boundaries in the app.

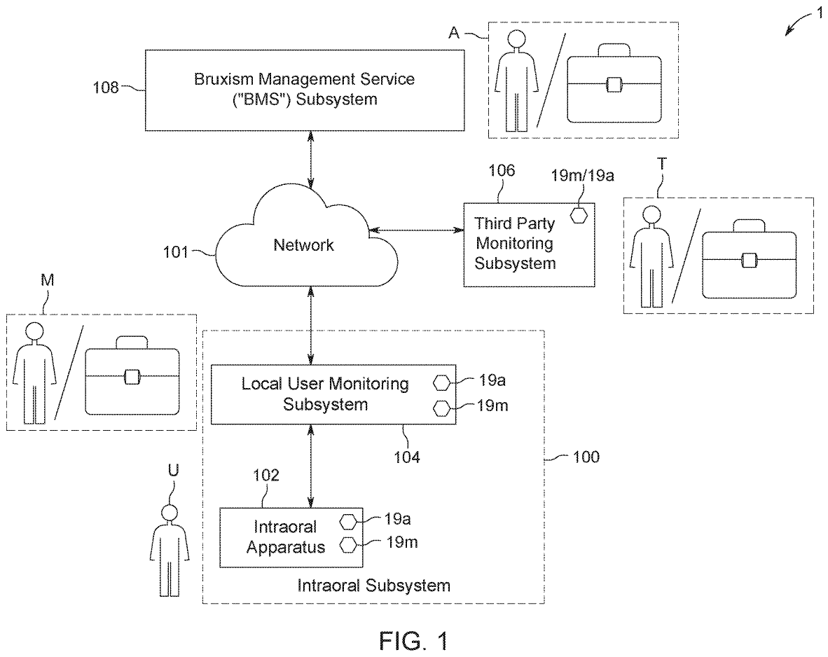

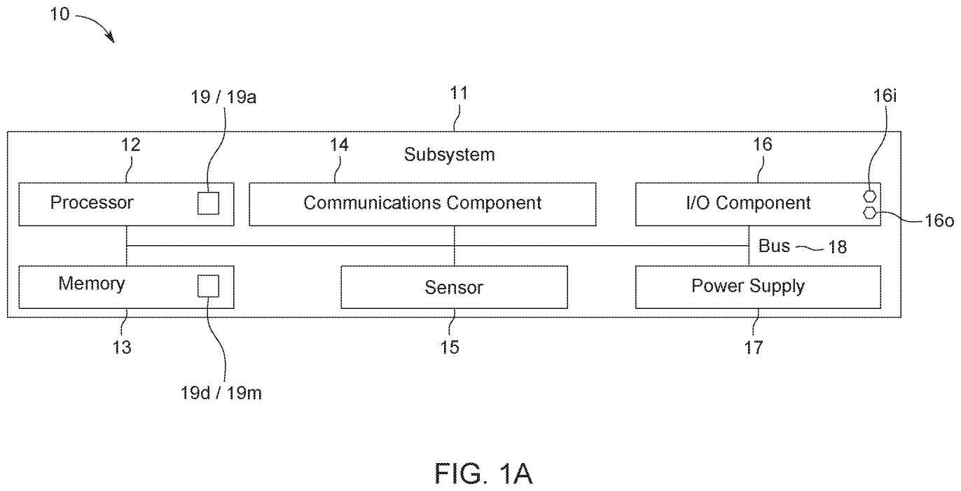

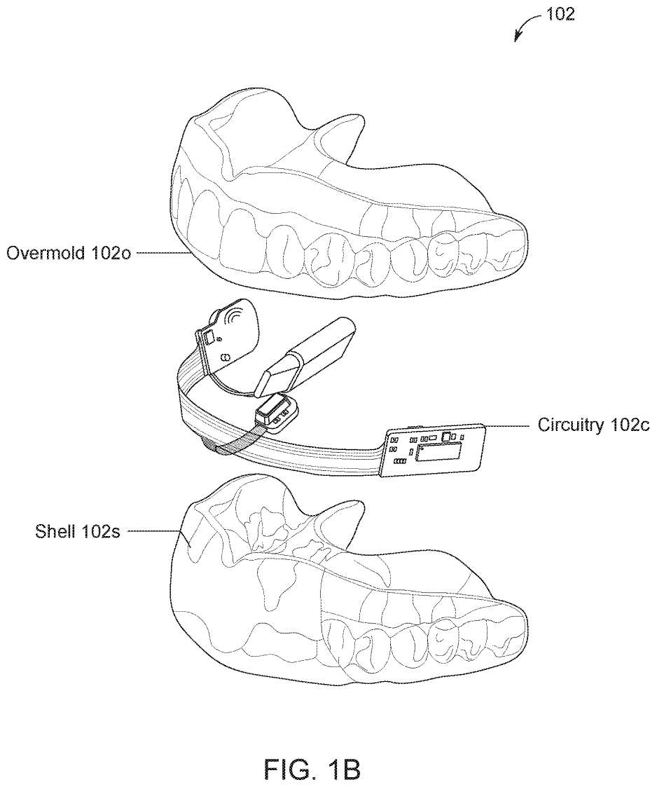

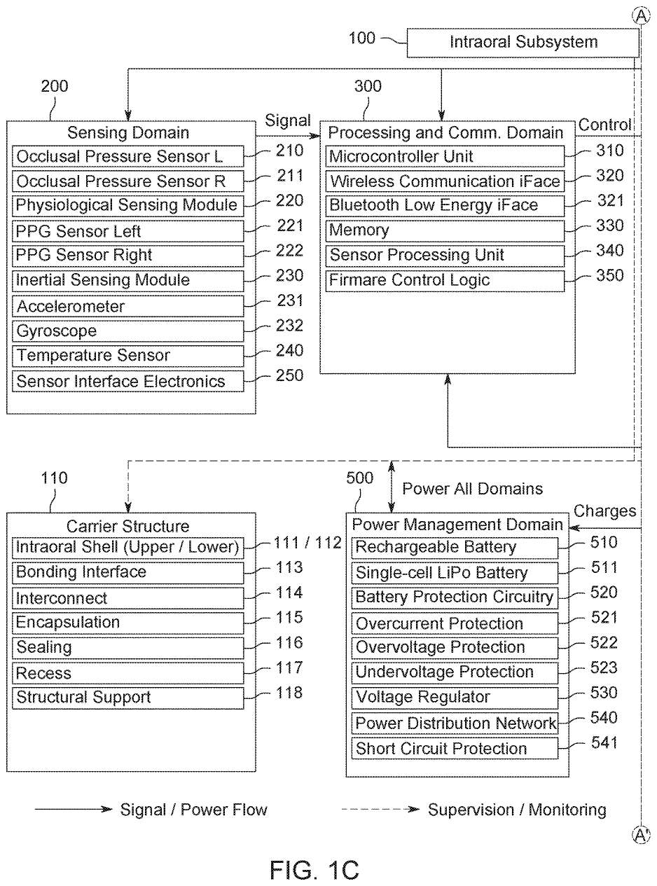

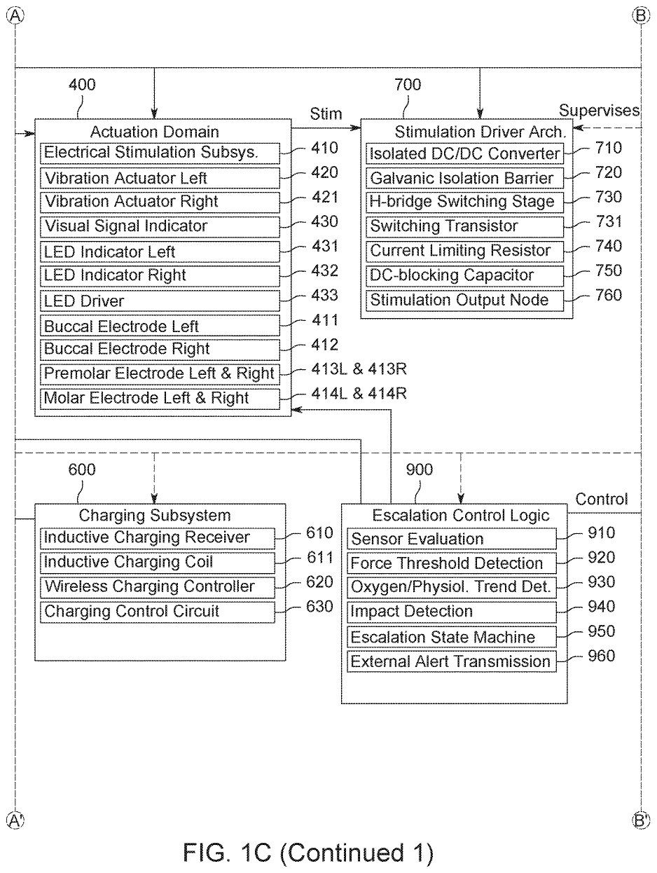

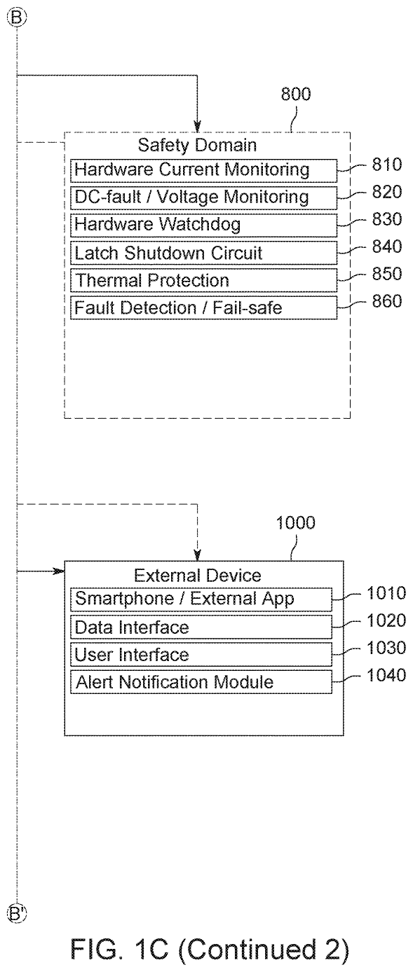

FIG. 1 is a schematic view of an illustrative system 1 in which managing bruxism may be facilitated utilizing one or more intraoral apparatuses 102, one or more local user monitoring subsystems 104, one or more third party monitoring subsystems 106, one or more bruxism management service (“BMS”) subsystems 108, and at least one communications network 101 through which any two or more subsystems of system 1, including apparatus 102, may communicate, either directly or indirectly. In some embodiments, intraoral apparatus 102 may be configured to communicate indirectly with subsystem 108 and/or subsystem 106 and/or otherwise through local user monitoring subsystem 104, which may act as a gateway device (e.g., using BLE or otherwise). In other embodiments, intraoral apparatus 102 may be configured to communicate directly, via communications network 101, with one or more of local user monitoring subsystem 104, third party monitoring subsystem 106, and/or bruxism management service subsystem 108. Direct communication may be achieved using any suitable wireless communication interface, including Bluetooth Low Energy, Wi-Fi, cellular relay, or any other network-enabled communication arrangements.

An intraoral apparatus 102 may be any mouthguard device or any other suitable intraoral device described herein, including, but not limited to, a dental guard, splint, orthodontic retainer form factor, orthodontic appliance, bite plate, partial dental structure, a custom shell configured for attachment to one or more teeth, and/or the like that may be worn by any suitable user U and that may be configured with any suitable circuitry.

A local user monitoring subsystem 104 may be any suitable subsystem that may be configured to be communicatively coupled to intraoral apparatus 102 (e.g., via BLE or any other suitable protocol) and run any suitable bruxism management system mobile app (e.g., a user's tablet, smartphone, laptop, etc.). Local user monitoring subsystem 104 may be operated, managed, or otherwise at least partially controlled by any suitable entity (e.g., a monitor M (e.g., user U or any entity that may wish to care for user U (e.g., a parent, coach, doctor, team computing system, etc.

Together, intraoral apparatus 102 and local user monitoring subsystem 104 may be referred to herein as any suitable intraoral (e.g., monitoring/neuromodulation) subsystem 100.

A third party monitoring subsystem 106 may be any suitable subsystem that may be configured to be communicatively coupled to local user monitoring subsystem 104 (e.g., via 5G, WiFi, and/or the like) for receiving any suitable patient data via the app (e.g., a patient's healthcare provider's server or smartphone or the like) and/or via any suitable subsystem of system 1. Third party monitoring subsystem 106 may be operated, managed, or otherwise at least partially controlled by any suitable entity (e.g., a third party T).

A BMS subsystem 108 may be any suitable subsystem that may be configured to (i) request and/or receive any suitable monitoring data associated with at least one apparatus 102 and/or (ii) access, configure, train, utilize, and/or otherwise manage any suitable bruxism management model(s) 19m (e.g., neural network(s) and/or learning engine(s), etc.) and/or generate, manage, host, and/or share any suitable bruxism management system application(s) 19a for communication with subsystem 104, subsystem 106, and/or apparatus 102 (e.g., via subsystem 104). BMS subsystem 108 may be operated, managed, or otherwise at least partially controlled by any suitable entity (e.g., an administrator A).

One, some, or each subsystem of system 1 may be configured communicate with any other one, some, or each subsystem of system I via any suitable wired and/or wireless communications network(s) (e.g., network 101). Network 101 may be the internet or any other suitable network, such that, when interconnected, a first subsystem may access information (e.g., monitoring data, controlling data, etc.) from a second subsystem as if such information were stored locally at that first subsystem. One, some, or each communications component or communications interface of a first subsystem and/or one, some, or each communications component or communications interface of a second subsystem may be a network interface that may include the mechanical, electrical, and/or signaling circuitry for communicating data over links (e.g., physical links) that may be coupled to network 101.

Although only a single one of each of intraoral apparatus 102 and subsystems 100, 104, 106, and 108 are shown in FIG. 1, system 1 may include two or more of one, some, or each of subsystems 100, 104, 106, and 108 and/or intraoral apparatus 102, and/or different networks 101 may be provided for enabling communication between different subsystems and/or apparatuses. Multiple intraoral apparatuses 102 may be monitored by a single local user monitoring subsystem 104, or each local user monitoring subsystem 104 may be utilized for monitoring only a single intraoral apparatus 102. In some embodiments, one or more subsystems of system 1 may be combined or omitted (e.g., a subsystem 104 and an apparatus 102 or a subsystem 106 may be combined into a single subsystem that may share one or more components and/or modules and/or other characteristics (e.g., location, memory, processing, power, etc.)).

As shown in FIG. 1A, a subsystem 10 of system 1 (e.g., one, some, or each of subsystems 100, 104, 106, and 108 and/or apparatus 102 of system 1 of FIG. 1) may include a processor component 12, a memory component 13, a communications component 14, a sensor 15, an input/output (“I/O”) component 16, a power supply component 17, a structure or housing 11, and/or a bus 18 that may provide one or more wired or wireless communication links or paths for transferring data and/or power to, from, or between various other components of subsystem 10. In some embodiments, one or more components of subsystem 10 may be combined or omitted. Moreover, subsystem 10 may include other components not combined or included in FIG. 1A and/or several instances of the components shown in FIG. 1A. For the sake of simplicity, only one of each of the components of subsystem 10 is shown in FIG. 1A.

I/O component 16 may include at least one input component 16i (e.g., button, mouse, keyboard, sensor (e.g., sensor 15), etc.) to receive information or other suitable data from a user/apparatus/subsystem/material/environment and/or at least one output component 160 (e.g., audio speaker, light, video display, haptic component, actuator, motor, heat/cooling component(s), etc.) to provide information or other suitable data to a user/apparatus/subsystem/material/environment, such as a touch screen that may receive input information through a user's touch of a display screen and that may also provide visual information to a user via that same display screen. Memory 13 may include one or more storage mediums, including for example, a hard-drive, flash memory, magnetic storage, permanent memory such as read-only memory (“ROM”), semi-permanent memory such as random access memory (“RAM”), any other suitable type of storage component, or any combination thereof (e.g., for storing data (e.g., data 19d (e.g., an application 19a and/or a management model 19m))). Memory 13 may include suitable logic, circuitry, and/or code that may enable storage of various types of information, such as received data, generated data, code, and/or configuration information.

Communications component 14 may be provided to allow subsystem 10 to communicate with one or more other subsystems 10 using any suitable communications protocol (e.g., via communications network 101). Communications component 14 can be operative to create or connect to a communications network (e.g., network 101). Communications component 14 can provide wireless communications using any suitable short-range or long-range communications protocol, such as Wi-Fi (e.g., an 802.11 protocol), Bluetooth, Bluetooth Low Energy Module (“BLE”), radio frequency systems (e.g., 1200 MHz, 2.4 GHz, and 5.6 GHz communication systems), near field communication (“NFC”), Zigbee, wireless local area network (“WLAN”), universal serial bus (“USB”), infrared, protocols used by wireless and cellular telephones and personal e-mail devices, or any other protocol supporting wireless communications. Communications component 14 can also be operative to connect to a wired communications network or directly to another data source wirelessly or via one or more wired connections. Communications component 14 may be a network interface that may include the mechanical, electrical, and/or signaling circuitry for communicating data over physical links that may be coupled to network 101. Such network interface(s) may be configured to transmit and/or receive any suitable data using a variety of different communication protocols, including, but not limited to, TCP/IP, UDP, ATM, synchronous optical networks (“SONET”), any suitable wireless protocols, Frame Relay, Ethernet, Fiber Distributed Data Interface (“FDDI”), and/or the like. In some embodiments, one, some, or each of such network interfaces may be configured to implement one or more virtual network interfaces, such as for Virtual Private Network (“VPN”) access.

Sensor 15 may be any suitable sensor that may be configured to sense any suitable data for subsystem 10 (e.g., location-based data via a GPS sensor system, image data, inertia or inertial data, motion data, environmental data, biometric data, external environment monitoring data, etc.). Sensor 15 may be a sensor assembly that may include any suitable sensor or any suitable combination of sensors operative to detect any suitable characteristic(s) of subsystem 10 and/or of a user thereof and/or of an associated apparatus (e.g., apparatus 102) and/or its environment/surroundings (e.g., a patient's mouth/teeth/jaw/etc.). Sensor 15 may include any suitable sensor(s), including, but not limited to, one or more of a GPS sensor, wireless communication sensor, image sensor, inertial sensor (e.g., inertial measurement unit (“IMU”)), accelerometer, directional sensor (e.g., compass), gyroscope, motion sensor, pedometer, passive infrared sensor, ultrasonic sensor, microwave sensor, a tomographic motion detector, camera, biometric sensor, light sensor, timer, and/or the like. Sensor 15 may include one or more image sensors for capturing video image data and/or still image data (e.g., sensor 15 may include a rear-facing camera and/or a front-facing camera and/or any other directional camera (e.g., on a gimballed and/or gyrostabilized platform and/or the like) and/or the like). Sensor 15 may include any suitable sensor components or subassemblies for detecting any suitable movement of subsystem 10 and/or of a user thereof and/or of an associated apparatus (e.g., apparatus 102). For example, sensor 15 may include one or more three-axis acceleration motion sensors (e.g., an accelerometer) that may be operative to detect linear acceleration in three directions (i.e., the x- or left/right direction, the y- or up/down direction, and the z- or forward/backward direction). As another example, sensor 15 may include one or more single-axis or two-axis acceleration motion sensors that may be operative to detect linear acceleration only along each of the x- or left/right direction and the y- or up/down direction, or along any other pair of directions. In some embodiments, sensor 15 may include an electrostatic capacitance (e.g., capacitance-coupling) accelerometer that may be based on silicon micro-machined micro electro-mechanical systems (“MEMS”) technology, including a heat-based MEMS type accelerometer, a piezoelectric type accelerometer, a piezo-resistance type accelerometer, and/or any other suitable accelerometer (e.g., which may provide a pedometer or other suitable function). Sensor 15 may be operative to directly or indirectly detect rotation, rotational movement, angular displacement, tilt, position, orientation, motion along a non-linear (e.g., arcuate) path, or any other non-linear motions. Additionally or alternatively, sensor 15 may include one or more angular rate, inertial, and/or gyro-motion sensors or gyroscopes for detecting rotational movement. For example, sensor 15 may include one or more rotating or vibrating elements, optical gyroscopes, vibrating gyroscopes, gas rate gyroscopes, ring gyroscopes, magnetometers (e.g., scalar or vector magnetometers), compasses, attitude sensors (e.g., for roll, pitch, yaw, etc.) and/or the like. Any other suitable sensors may also or alternatively be provided by sensor 15 for detecting motion or otherwise at or with subsystem 10, such as any suitable pressure sensors, altimeters, flow sensors, spin sensors, temperature sensors, thermocouples, odor sensors, gas sensors, fluid sensors, humidity sensors, opacity sensors, actuator position sensors, belt rate sensors, electrical resistivity sensors, real density sensors, and/or the like. Using sensor 15, subsystem 10 may be configured to determine a velocity, acceleration, orientation, and/or any other suitable motion attribute of subsystem 10 or an associated apparatus (e.g., apparatus 102). Sensor 15 may include any suitable sensor components or subassemblies for detecting any suitable biometric data and/or health data and/or the like of a user of user subsystem 10. For example, sensor 15 may include any suitable biometric sensor that may include, but is not limited to, one or more facial recognition sensors, fingerprint scanners, iris scanners, retinal scanners, voice recognition sensors, gait sensors, hair sensors, hand geometry sensors, signature scanners, keystroke dynamics sensors, vein matching sensors, heart beat sensors, body temperature sensors, odor or scent sensors, behavioral biometric sensors (e.g., behavioral modeling of movement, orientation, gesture, pausality, etc.), DNA sensors, sensors for any unclonable or extremely difficult to replicate personal function, and/or any other suitable sensors for detecting any suitable metrics related to any suitable characteristics of a user, which may also include health-related optical sensors, capacitive sensors, thermal sensors, electric field (“eField”) sensors, and/or ultrasound sensors, such as photoplethysmogram or photoplethysmography (“PPG”) sensors, electrocardiogramsors, EMG sensors, force sensors, galvanic skin response (“GSR”) sensors, posture sensors, stress sensors, and/or the like. Sensor 15 may include a microphone, camera, scanner (e.g., a barcode scanner or any other suitable scanner that may obtain product identifying information from a code, such as a linear barcode, a matrix barcode (e.g., a quick response (“QR”) code), or the like), proximity sensor, light detector, temperature sensor, motion sensor, biometric sensor (e.g., a fingerprint reader or other feature (e.g., facial) recognition sensor, which may operate in conjunction with a feature-processing application that may be accessible to subsystem 10 for attempting to authenticate a user), line-in connector for data and/or power, and/or combinations thereof. In some examples, each sensor can be a separate device, while, in other examples, any combination of two or more of the sensors can be included within a single device. For example, a gyroscope, accelerometer, PPG sensor, GSR sensor, and temperature sensor can be included within a wearable subsystem 10, such as a smart watch, while a scale, blood pressure cuff, blood glucose monitor, SpO2 sensor, respiration sensor, posture sensor, stress sensor, and asthma inhaler can each be separate devices. Motion sensor(s) may be used to facilitate movement and orientation related functions of subsystem 10 and/or associated apparatus, for example, to detect movement, direction, and/or orientation of subsystem 10. While specific examples are provided, it should be appreciated that other sensors can be used and other combinations of sensors can be combined into a single subsystem 10. Sensor 15 may include any suitable sensor components or subassemblies for detecting any suitable characteristics of any suitable condition of the lighting of the environment of subsystem 10 or an associated apparatus or material. For example, sensor 15 may include any suitable light sensor that may include, but is not limited to, one or more ambient visible light color sensors, illuminance ambient light level sensors, ultraviolet (“UV”) index and/or UV radiation ambient light sensors, and/or the like. Any suitable light sensor or combination of light sensors may be provided for determining the illuminance or light level of ambient light in the environment of subsystem 10 (e.g., in lux or lumens per square meter, etc.) and/or for determining the ambient color or white point chromaticity of ambient light in the environment of subsystem 10 (e.g., in hue and colorfulness or in x/y parameters with respect to an x-y chromaticity space, etc.) and/or for determining the UV index or UV radiation in the environment of subsystem 10 (e.g., in UV index units, etc.). A suitable light sensor may include, for example, a photodiode, a phototransistor, an integrated photodiode and amplifier, or any other suitable photo-sensitive device. In some embodiments, more than one light sensor may be integrated into subsystem 10. Sensor 15 may include any suitable sensor components or subassemblies for detecting any suitable characteristics of any suitable condition of the air quality of the environment of subsystem 10 or of an associated apparatus and/or material. For example, sensor 15 may include any suitable air quality sensor that may include, but is not limited to, one or more ambient air flow or air velocity meters, ambient oxygen level sensors, volatile organic compound (“VOC”) sensors, ambient humidity sensors, ambient temperature sensors, and/or the like. Any suitable ambient air sensor or combination of ambient air sensors may be provided for determining the oxygen level of the ambient air in the environment of subsystem 10 (e.g., in O₂% per liter, etc.) and/or for determining the air velocity of the ambient air in the environment of subsystem 10 (e.g., in kilograms per second, etc.) and/or for determining the level of any suitable harmful gas or potentially harmful substance (e.g., VOC (e.g., any suitable harmful gasses, scents, odors, etc.) or particulate or dust or pollen or mold or the like) of the ambient air in the environment of subsystem 10 (e.g., in HG % per liter, etc.) and/or for determining the humidity of the ambient air in the environment of subsystem 10 (e.g., in grams of water per cubic meter, etc. (e.g., using a hygrometer)) and/or for determining the temperature of the ambient air in the environment of subsystem 10 (e.g., in degrees Celsius, etc. (e.g., using a thermometer)). Sensor 15 may include any suitable sensor components or subassemblies for detecting any suitable characteristics of any suitable condition of the sound quality of the environment of subsystem 10 and/or associated apparatus and/or material. For example, sensor 15 may include any suitable sound quality sensor that may include, but is not limited to, one or more microphones or the like that may determine the level of sound pollution or noise in the environment of subsystem 10 (e.g., in decibels, etc.). Sensor 15 may also include any other suitable sensor for determining any other suitable characteristics about a user of subsystem 10 and/or the environment of subsystem 10 and/or apparatus and/or material associated with subsystem 10. In some examples, different sensors can be placed in different locations inside or on the surfaces of subsystem 10 (e.g., some located inside housing 11 and some attached to an attachment mechanism (e.g., a wrist band coupled to a housing of a wearable device), or the like). In other examples, one or more sensors can be worn by a user separately as different parts of a single subsystem 10 or as different devices (e.g., for associating with different respective components of an associated apparatus (e.g., apparatus 102)). In such cases, the sensors can be configured to communicate with subsystem 10 using a wired and/or wireless technology (e.g., via communications component 14). In some examples, sensors can be configured to communicate with each other and/or share data collected from one or more sensors.

Power supply 17 can include any suitable circuitry for receiving and/or generating power, and for providing such power to one or more of the other components of subsystem 10.

For example, power supply assembly 17 can be coupled to a power grid (e.g., when subsystem 10 is not acting as a portable device or when a battery of the subsystem is being charged at an electrical outlet with power generated by an electrical power plant). As another example, power supply assembly 17 may be configured to generate power from a natural source (e.g., solar power using solar cells). As another example, power supply assembly 17 can include one or more batteries for providing power (e.g., when subsystem 10 is acting as a portable device). Subsystem 10 may also be provided with a housing 11 that may at least partially enclose one or more of the components of subsystem 10 for protection from debris and other degrading forces external to subsystem 10. Each component of subsystem 10 may be included in the same housing 11 (e.g., as a single unitary device, such as a portable media device or server or mouthguard) and/or different components may be provided in different housings (e.g., a keyboard input component may be provided in a first housing that may be communicatively coupled to a processor component and a display output component that may be provided in a second housing, such as in a desktop computer set-up).

Processor 12 may be used to run one or more applications, such as an application 19 that may be accessible from memory 13 (e.g., as a portion of data 19d) and/or any other suitable source (e.g., from network 101 or any other subsystem and an active internet or other suitable data connection). Application 19 may include, but is not limited to, one or more operating system applications, firmware applications, communication applications (e.g., for enabling communication of data between subsystems 10), third party service applications (e.g., sensor applications, social media applications, etc.), internet browsing applications (e.g., for interacting with a website provided by a third party subsystem or other subsystem for enabling subsystem 10 to interact with an online service), application programming interfaces (“APIs”), software development kits (“SDKs”), BMS applications (e.g., a web application or a native application that may be at least partially produced by BMS subsystem 108 or otherwise for enabling subsystem 10 to interact with an online service), or any other suitable applications (e.g., a BMS application). For example, processor 12 may load an application 19 as an interface program to determine how instructions or data received via an input component of I/O component 16 or other component of subsystem 10 (e.g., sensor 15 and/or communications component 14) may manipulate the way in which information may be stored (e.g., in memory 13) and/or provided to the user or associated apparatus via an output component of I/O component 16 and/or to another subsystem via communications component 14. As one example, application 19 may provide a user or subsystem 10 with the ability to interact with a BMS platform (“BMSP”) of system 1, where application 19 may be a third party application that may be running on subsystem 10 (e.g., an application associated with BMS subsystem 108 and/or a third party subsystem or the like) that may be loaded on subsystem 10 (e.g., using communications component 14) via an application market, such as the Apple App Store or Google Play, or that may be accessed via an internet application or web browser (e.g., by Apple Safari or Google Chrome) that may be running on subsystem 10 and that may be pointed to a uniform resource locator (“URL”) whose target or web resource may be managed by or otherwise affiliated with the BMSP. Processor 12 may include suitable logic, circuitry, and/or code that may enable processing data and/or controlling operations of subsystem 10. In this regard, processor 12 may be enabled to provide control signals to various other components of subsystem 10. Processor 12 may also control transfers of data between various portions of subsystem 10. Processor 12 may further implement an operating system or may otherwise execute code to manage operations of subsystem 10.

Subsystem 10 may be configured to have any physical structure (e.g., by one or more housings 11) that may include, but is not limited to, any suitable portable, mobile, wearable, implantable, rideable, controllable, or hand-held mobile electronic device (e.g., a portable and/or handheld media player), a headset, a helmet, glasses, a wearable, an intraoral mouthguard, an earbud or other ear wearable device, a tablet computer, a laptop computer, a controller, a virtual reality (“VR”) and/or augmented reality (“AR”) and/or mixed reality (“MR”) device, a vehicle, server, sensor system, actuator system, and/or any other machine or device or housing or structure that can be utilized to manage variables of an apparatus (e.g., apparatus 102). Alternatively, subsystem 10 may not be portable during use, but may instead be generally stationary (e.g., as a type of BMS subsystem 108). Not all of the depicted components may be used in all implementations, however, and one or more implementations may include additional or different components than those shown in FIG. 1A. In one or more implementations, one or more of processor 12, memory 13, sensor(s) 15, communications interface or communications component 14, I/O component 16, and/or power supply 17, and/or one or more portions thereof, may be implemented in software (e.g., subroutines and code), may be implemented in hardware (e.g., an application specific integrated circuit (“ASIC”), a field programmable gate array (“FPGA”), a programmable logic device (“PLD”), a controller, a state machine, gated logic, discrete hardware components, or any other suitable devices), and/or a combination of both. Variations in the arrangement and type of the components may be made without departing from the spirit or scope of the claims as set forth herein. Additional components, different components, or fewer components may be provided.

FIG. 1B shows one particular implementation of intraoral apparatus 102, which may include a smart wearable (e.g., mouthguard) with an intraoral shell 102s and any suitable circuitry 102c (e.g., board(s), microcontroller(s), sensor(s), driver(s), battery(ies), etc.) that may be incorporated into or otherwise coupled to shell 102s, which may be provided with any suitable intraoral overmold 1020. In some embodiments, circuitry 102c (e.g., intraoral circuitry) may be implemented as a continuous band that may be provided along the entire curvature of shell 102s.

FIG. 1C illustrates an exemplary overall system architecture of an intraoral subsystem 100 (e.g., an intraoral monitoring/neuromodulation device or system) of the BMSP of the disclosure. As shown, intraoral subsystem 100 may include and/or support any suitable interaction between various components, including, but not limited to, a sensing domain 200, a processing and communication domain 300, an actuation domain 400, a power management domain 500, a charging subsystem 600, a stimulation driver architecture 700, a safety domain 800, escalation control logic 900, a carrier structure 110, an external device 1000, and/or the like.

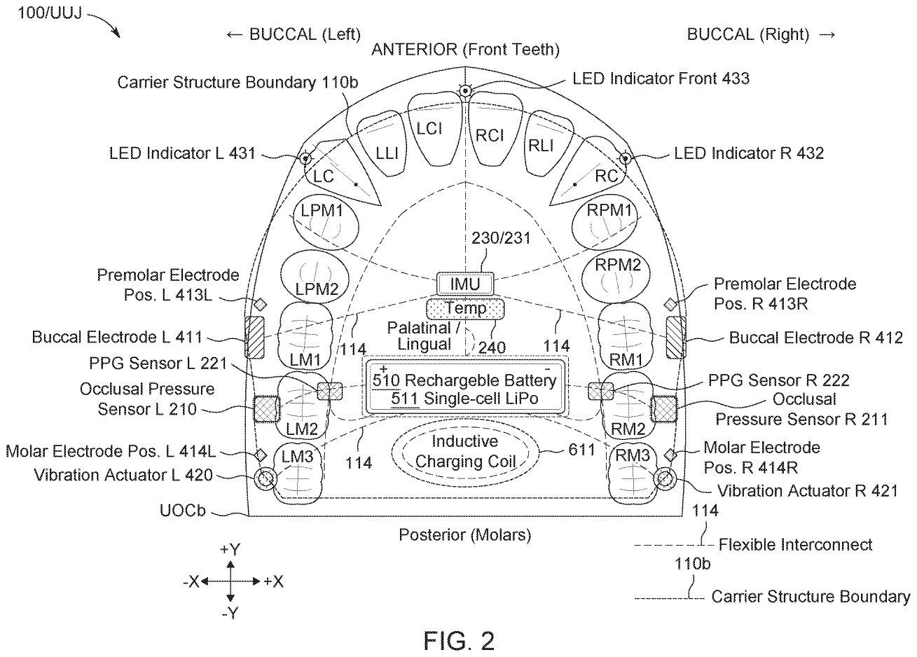

As shown in FIG. 1C, sensing domain 200, which may be referred to herein as a sensing subsystem, may be configured to send any suitable signal(s) to processing and communication domain 300 and/or to receive any suitable power from power management domain 500. Sensing domain 200 may include any suitable features, including, but not limited to, a left occlusal pressure sensor 210 (e.g., any suitable sensor 15, force sensor, pressure sensor, occlusal force sensing element, force sensing element, pressure sensor, pressure sensor data (or generator thereof), piezoelectric element, piezoresistive element, strain gauge, capacitive force sensor, force-sensitive resistor, etc.), a right occlusal pressure sensor 211 (e.g., any suitable sensor 15, force sensor, pressure sensor, occlusal force sensing element, force sensing element, pressure sensor, pressure sensor data (or generator thereof), piezoelectric element, piezoresistive element, strain gauge, capacitive force sensor, force-sensitive resistor, etc.), a physiological sensing module 220 (e.g., any suitable sensor 15, physiological sensor(s), physiological indicator sensor(s), physiological sensing element(s), any suitable impedance sensor(s), optical sensor(s), proxy sensor(s), force sensor(s), inertial sensor(s), temperature sensor(s), acoustic sensor(s), etc.), a left PPG sensor 221 (e.g., any suitable sensor 15, physiological sensing element, physiological indicator sensor, optical sensor, etc. (e.g., of module 220)), a right PPG sensor 222 (e.g., any suitable sensor 15, physiological sensing element, physiological indicator sensor, optical sensor, etc. (e.g., of module 220)), an inertial sensing module 230 (e.g., any suitable sensor 15, inertial sensing element, combined inertial measurement unit, etc.), an accelerometer 231 (e.g., any suitable sensor 15, acceleration sensing element, acceleration sensor, multi-axis accelerometer, inertial sensing element, etc. (e.g., of module 230)), a gyroscope 232 (e.g., any suitable sensor 15, gyroscopic sensor, multi-axis gyroscope, inertial sensing element, etc. (e.g., of module 230)), a temperature sensor 240 (e.g., any suitable sensor 15, temperature monitoring circuitry, physiological indicator sensor, etc. (e.g., of module 220)), any suitable sensor interface electronics 250, and/or the like. Additional sensing elements (e.g., one or more additional sensors 15) may include, but are not limited to, one or more electromyography (“EMG”) sensors that may be configured to detect muscle activity associated with masticatory muscles, one or more galvanic skin response (“GSR”) sensors or impedance-based sensors that may be adapted for intraoral tissue conductivity measurement and/or configured to detect physiological stress or tissue conductivity changes, one or more acoustic sensors (e.g., microphones, vibration pickups, etc.) that may be configured to detect any suitable intraoral or tooth contact sounds (e.g., tooth grinding, clicking, impact-related sound signatures, etc.), one or more capacitive proximity sensors that may be configured to detect intraoral positioning and/or wear state, one or more bioimpedance sensors that may be configured to detect tissue contact and/or wear-state validation (e.g., to assess tissue contact, hydration, perfusion-related parameters, etc.), one or more pressure distribution sensor arrays that may be configured to map spatial occlusal load distribution, one or more airflow or respiration-related sensors that may be configured to detect breathing patterns and/or airflow variations, one or more chemical or pH sensors that may be configured to detect intraoral environmental changes, and/or the like. Such sensor(s) may be integrated within sensing domain 200 and may be used individually or in combination for enhanced multi-modal event detection and classification. It is to be noted that only a subset of representative sensors is illustrated for clarity, and additional sensors may be integrated within carrier structure 110 of FIG. 2 without being explicitly shown.

As shown in FIG. 1C, processing and communication (“P&C”) domain 300, which may be referred to herein as a communication domain, a processing domain, a processing subsystem, a sensing and control subsystem, a low-voltage sensing and processing domain, and/or the like, may be configured to receive any suitable signal(s) from sensing domain 200 and/or from escalation control logic 900 and/or to send any suitable control signal(s) to actuation domain 400 and/or to receive any suitable power from power management domain 500. P&C domain 300 may include any suitable features, including, but not limited to, a microcontroller unit 310 (e.g., any suitable processor 12, processing subsystem, etc.), a wireless communication interface 320 (e.g., any suitable communications component 14, wireless communication link, etc.), a Bluetooth Low Energy (“BLE”) interface 321 (e.g., any suitable communications component 14, wireless communication interface, wireless communication link, etc. (e.g., of interface 320)), a memory 330 (e.g., any suitable memory 13, memory processing subsystem configured to store event data and/or data associated with an impact-related event (e.g., timestamps, severity score, peak force, peak acceleration, peak occlusal force, event duration, device intervention state, and/or the like) and/or to store a post-event record associated with a sensor processing unit, and/or the like), a sensor processing unit 340 (e.g., any suitable processor 12, processing subsystem that may be configured to compute at least one bruxism-relevant metric (e.g., accumulated occlusal force exposure, peak-to-average ratio, event duration, event frequency, burst density, right/left asymmetry, inter-event timing, and/or the like), etc. (e.g., of unit 310)), firmware control logic 350 (e.g., software control), and/or the like.

As shown in FIG. 1C, actuation domain 400, which may be referred to herein as an intervention subsystem, may be configured to receive any suitable control signal(s) from P&C domain 300 and/or from escalation control logic 900 and/or to send any suitable stimulation signal(s) to actuation domain 400 and/or to receive any suitable power from power management domain 500. Actuation domain 400 may include any suitable features, including, but not limited to, an electrical stimulation subsystem 410 (e.g., any suitable output component 160 (e.g., a vibration output and/or a neuromodulation stimulation output and/or a generator thereof) and/or processing circuitry), a left buccal electrode 411 (e.g., any suitable output component 160, any suitable electrode, electrode placement, electrode contact surface, intraoral electrode (e.g., positioned on any suitable buccal surface (e.g., left buccal surface), field shaped toward any suitable masticatory target zone, etc.), etc.), a right buccal electrode 412 (e.g., any suitable output component 160, any suitable electrode, electrode placement, electrode contact surface, intraoral electrode (e.g., positioned on any suitable buccal surface (e.g., right buccal surface), field shaped toward any suitable masticatory target zone, etc.), etc.), a left premolar-region electrode 413L (e.g., any suitable output component 160, any suitable electrode, electrode placement, electrode contact surface, intraoral electrode (e.g., positioned (e.g., adjacent a left premolar) to define the longitudinal extent of a stimulation field), etc.), a right premolar-region electrode 413R (e.g., any suitable output component 160, any suitable electrode, electrode placement, electrode contact surface, intraoral electrode (e.g., positioned (e.g., adjacent a left premolar) to define the longitudinal extent of a stimulation field), etc.), a left molar-region electrode 414L (e.g., any suitable output component 160, any suitable electrode, electrode placement, electrode contact surface, intraoral electrode (e.g., positioned (e.g., adjacent a left molar) to define the longitudinal extent of a stimulation field), etc.), a right molar-region electrode 414R (e.g., any suitable output component 160, any suitable electrode, electrode placement, electrode contact surface, intraoral electrode (e.g., positioned (e.g., adjacent a right molar) to define the longitudinal extent of a stimulation field), etc.), a left vibration actuator 420 (e.g., any suitable output component 160, vibration output and/or generator thereof, micro-motor vibration output and/or generator thereof, haptic actuator, piezoelectric vibration actuator, etc.), a right vibration actuator 421 (e.g., any suitable output component 160, vibration output and/or generator thereof, micro-motor vibration output and/or generator thereof, haptic actuator, piezoelectric vibration actuator, etc.), a visual signal indicator 430 (e.g., any suitable output component 160, visual signaling component, visual signaling information and/or generator thereof, visible signal and/or generator thereof, externally visible advisory status flag and/or generator thereof, etc.), a left light-emitting diode (“LED”) indicator 431 (e.g., any suitable output component 160, light, LED, warning LED, visual signaling component, light-emitting element, visible signal and/or generator thereof, externally visible advisory status flag and/or generator thereof, etc. (e.g., of indicator 430)), a right LED indicator 432 (e.g., any suitable output component 160, light, LED, warning LED, visual signaling component, light-emitting element, visible signal and/or generator thereof, externally visible advisory status flag and/or generator thereof, etc. (e.g., of indicator 430)), an LED driver 433 (e.g., front LED indicator (e.g., any suitable output component 160, light, LED, warning LED, visual signaling component, light-emitting element, visible signal and/or generator thereof, externally visible advisory status flag and/or generator thereof, etc. (e.g., of indicator 430))), and/or the like. Electrical stimulation subsystem 410 may include one or more controlled current and/or voltage sources that may be configured to generate biphasic, charge-balanced stimulation pulses. The neuromodulation stimulation may include biphasic, charge-balanced electrical pulses, including pulsed stimulation sequences configured to minimize DC offset and electrode polarization. Electrical stimulation subsystem 410 may include any suitable pulse shaping circuitry, waveform generation circuits, switching networks (e.g., H-bridge configurations), electrode routing or multiplexing elements, and/or the like. In some embodiments, electrical stimulation subsystem 410 may be implemented using any suitable constant-current stimulators, programmable pulse generators, pulse-width modulation circuits, and/or galvanically isolated stimulation drivers that may be configured to operate within a defined stimulation domain. Electrical stimulation subsystem 410 may be configured to provide controlled neuromodulation of intraoral and/or adjacent neuromuscular pathways. Such neuromodulation may include modulation of sensory afferent pathways, reflex pathway modulation, interruption or reduction of bruxism-related activity, inhibition of excessive muscle activation, and/or the like. In some embodiments, electrical stimulation subsystem 410 may provide stimulation that may be configured to influence neuromuscular signaling without inducing a direct or sustained muscle contraction, and/or may operate any suitable below motor activation threshold(s). Any suitable electrode(s) (e.g., one or more of electrodes 411-414R) may provide a physical interface through which stimulation signals generated by electrical stimulation subsystem 410 may be delivered to the tissue of a patient user. For example, such electrode(s) may represent terminal output elements of intervention subsystem or actuation domain 400. In some embodiments, feature 433 may correspond to an LED driver (see, e.g., FIG. 1C) and to a front-facing LED indicator (see, e.g., FIG. 2), wherein such LED driver circuitry may be integrated within an actuation domain and electrically control one or more externally visible LED elements. Additional actuators of intraoral subsystem 100 (e.g., of actuation domain 400) may include, but are not limited to, one or more acoustic transducers for generating audible signals (e.g., miniature speakers, bone conduction transducers, etc.), one or more haptic actuators based on piezoelectric or linear resonant actuator technology (e.g., linear resonant actuators, piezo haptic elements, etc.), one or more electro-tactile stimulation elements, one or more thermal feedback elements for temperature-based signaling (e.g., thermal actuators (e.g., controlled heating elements for sensory feedback)), one or more other optical indicators beyond LEDs (e.g., light guides, diffused illumination elements, etc.), one or more multi-modal feedback units combining vibration, electrical stimulation, and visual signaling, and/or the like. Such actuators may be integrated within actuation domain 400 and/or distributed along carrier structure 110 (e.g., depending on functional requirements). It is to be noted that only a subset of representative actuators is illustrated for clarity, and additional actuators may be integrated within carrier structure 110 of FIG. 2 without being explicitly shown.

As shown in FIG. 1C, power management domain 500, which may be referred to herein as a low-voltage sensing and processing domain, sensing and control subsystem, and/or the like, may be configured to be charged by charging subsystem 600 and/or to provide power to one, some, or all suitable component(s) of FIG. 1C. Power management domain 500 may include any suitable features, including, but not limited to, a battery 510 (e.g., any suitable power supply 17, rechargeable battery, single-cell lithium-based rechargeable battery, etc.), a single-cell lithium polymer (“LiPo”) battery 511 (e.g., any suitable power supply 17, a lithium-based rechargeable battery, etc.), battery protection circuitry 520 (e.g., any suitable power supply 17, any suitable battery protection component(s), etc.), any suitable over-current protection 521 (e.g., any suitable power supply 17, any suitable over-current protection circuitry (e.g., of circuitry 520), etc.), any suitable over-voltage protection 522 (e.g., any suitable power supply 17, any suitable over-voltage protection circuitry (e.g., of circuitry 520), etc.), any suitable under-voltage protection 523 (e.g., any suitable power supply 17, any suitable under-voltage protection circuitry (e.g., of circuitry 520), etc.), any suitable voltage regulator 530, any suitable power distribution network 540 (see, e.g., power distribution lines 1173, etc.), any suitable short-circuit protection 541, and/or the like.

As shown in FIG. 1C, charging subsystem 600, which may be referred to herein as a wireless charging subsystem (see, e.g., wireless charging receiver 1161, wireless charging system 1243, etc.), may be configured to charge power management domain 500. Charging subsystem 600 may include any suitable features, including, but not limited to, an inductive charging receiver 610 (e.g., any suitable power supply 17, wireless charging receiver, etc.), any suitable inductive charging coil 611 (e.g., any suitable power supply 17), any suitable wireless charging controller 620, any suitable charging control circuit 630 (e.g., any suitable power supply 17, wireless charging control circuitry, etc.), and/or the like.

As shown in FIG. 1C, stimulation driver architecture 700, which may be referred to herein as a stimulation subsystem, may be configured to be stimulated by actuation domain 400, supervised by safety domain 800, and/or powered by power management domain 500. Stimulation driver architecture 700 may include any suitable features, including, but not limited to, any suitable direct current (“DC”)/DC converter 710 (e.g., any suitable isolated DC/DC converter, isolated supply, isolated converter, hardware safety circuitry, isolated power source, isolated conversion component, transformer-based isolated converter, low-voltage sensing and processing domain, stimulation subsystem, etc.), any suitable galvanic isolation barrier 720 (e.g., any suitable hardware safety circuitry, isolation barriers, low-voltage sensing and processing domain, and/or the like that may be configured to define (i) a low-voltage domain (e.g., a low-voltage processing domain 791 of an electrical architecture 790 for dual-domain isolation) that may include a sensing and control subsystem (e.g., domain(s) 200, 300, 500, etc.) and (ii) an isolated stimulation domain (e.g., a galvanically isolated stimulation domain 799 of architecture 790) that may include a stimulation subsystem (e.g., electrical stimulation subsystem 410, isolated supply 710, etc.), etc.), an H-bridge switching stage 730 (e.g., any suitable discrete switching elements), any suitable switching transistor 731 (e.g., any suitable discrete switching elements), any suitable current limiting resistor 740 (e.g., any suitable independent series resistance), any suitable DC-blocking capacitor 750 (e.g., any suitable DC-blocking components, DC-blocker, DC-block capacitor, etc.), any suitable stimulation output node 760, and/or the like. Stimulation driver architecture 700 may represent an implementation of electrical stimulation subsystem 410. Subsystem 410 may define the functional capability of delivering neuromodulation stimulation, whereas architecture 700 may illustrate one possible hardware realization of such functionality.

As shown in FIG. 1C, safety domain 800, which may be referred to herein as a hardware safety domain, hardware-enforced limits generator, safety subsystem, hardware safety enforcement subsystem, and/or the like, may be configured to supervise stimulation driver architecture 700 and/or be powered by power management domain 500. Safety domain 800 may include any suitable features, including, but not limited to, any suitable hardware current monitor 810 (e.g., any suitable hardware for current monitoring, hardware safety circuitry, current limiting hardware, hardware-enforced limits, hardware current limiting circuits, independent comparator threshold or enforcer thereof, hard current limit or enforcer thereof, and/or the like), any suitable component(s) 820 for DC-fault/voltage monitoring (e.g., any suitable DC-fault monitor, DC-fault, voltage monitoring, hardware safety circuitry, DC-fault detection, hardware-enforced limits, hardware voltage monitoring circuits, DC-blocking capacitor, DC-fault detection circuit, unsafe-condition detector, and/or the like), any suitable hardware watchdog 830 (e.g., any suitable hardware safety circuitry, hardware-enforced limits, watchdog supervision interval, watchdog circuit, unsafe-condition detector, watchdog, and/or the like, any suitable latch shutdown circuit 840 (e.g., any suitable hardware safety circuitry, latch-based shutdown, hardware-enforced limits, latch circuits, latch-based shut-down path, latched shut-down mechanism, latch shutdown, fault-triggered stimulation disable state, latched shut-down state, fault latch, and/or the like), any suitable thermal protection 850 (e.g., any suitable temperature monitoring circuitry), any suitable component(s) 860 for fault detection/fail-safe (e.g., fail-safe disable, fail-safe disable logic, hardware-enforced limits, unsafe-condition detector, and/or the like), and/or the like. Safety domain 800 may be illustrated in FIG. 1C using a broken line to indicate functional independence from firmware-controlled domains and to emphasize that safety enforcement may operate as a separate hardware-supervised domain that cannot be overridden by software execution. Certain coupling lines of FIG. 1C may be illustrated using a broken line to indicate supervision and/or monitoring, while other coupling lines may be illustrated using a solid line to indicate signal and/or power flow.

As shown in FIG. 1C, escalation control logic 900, which may be referred to herein as a software control, escalation controller, and/or the like, may be configured to provide any suitable control signal(s) to external device 1000 and/or P&C domain 300 and/or actuation domain 400 and/or be powered by power management domain 500. Escalation control logic 900 may include any suitable features, including, but not limited to, any suitable sensor evaluation component 910, force threshold detection component 920, oxygen/physiological trend detection component 930, impact detection component 940, escalation state machine component 950 (e.g., any suitable escalation controller or central deterministic logic (e.g., for normal monitoring (e.g., no intervention), vibration (e.g., vibrating actuator(s) 420/421), stimulation (e.g., stimulating with electrical stimulation subsystem 410), combined vibration and stimulation, cooldown/lockout (e.g., timed recovery), etc.)), external alert transmission component 960, and/or the like.

As shown in FIG. 1C, external device 1000, which may be referred to herein as an external computing device, local user monitoring subsystem (e.g., subsystem 104), and/or the like, may be configured to receive any suitable control signal(s) from escalation control logic 900 and/or be powered by power management domain 500. External device 1000 may correspond to local user monitoring subsystem 104 of FIG. 1, although additional external devices may also be supported. External device 1000 may be configured not only to receive (e.g., any suitable control signal(s) from escalation control logic 900 and/or power from power management domain 500) but also to transmit data to other portions of subsystem 100 and/or system 1 (e.g., for initially configuring, updating, or customizing the use of the intraoral device), including, but not limited to, configuration parameters, firmware updates, threshold adjustments, personalization settings, intervention parameter tuning, and/or the like. Such bidirectional communication may occur via any suitable interface(s), including any suitable wireless interfaces, such as BLE, Wi-Fi, and/or other communication protocols. External device 1000 may include any suitable features, including, but not limited to, any suitable smartphone/external application 1010 (e.g., any suitable application(s) 19a, external application interaction, external application, application interface, smartphone app, etc.), any suitable data interface 1020 (e.g., any suitable communications component 14, I/O component 16, etc.), any suitable user interface 1030 (e.g., any suitable communications component 14, I/O component 16, etc., such as an advisory display for a coach/trainer, application interface, and/or the like), any suitable alert notification module 1040, and/or the like. In some embodiments, data interface 1020 may include any suitable wireless transceivers, Bluetooth modules, USB interfaces, cloud connectivity interfaces, and/or the like. In some embodiments, user interface 1030 may include any suitable graphical user interfaces, dashboards, mobile application displays, clinician monitoring panels, coaching interfaces, and/or the like. In some embodiments, alert notification module 1040 may generate or provide any suitable push notifications, visual alerts, haptic alerts, audible alarms, cloud-based alert forwarding, and/or the like (e.g., to any suitable third-party systems (e.g., any suitable third party monitoring subsystem 106)).

As shown in FIG. 1C, carrier structure 110, which may be referred to herein as a carrier plate or tooth-supported or dental-arch supported intraoral body or otherwise, may be provided (e.g., for defining a carrier boundary that may be routed to match or support any suitable module placeholders). Carrier structure 110 may include or be provided with any suitable features, including, but not limited to, an intraoral shell (e.g., an upper intraoral shell 111 (e.g., any suitable intraoral shell 102s, upper jaw shell (e.g., for maxillary variants), etc.) and/or a lower intraoral shell 112 (e.g., any suitable intraoral shell 102s, lower jaw shell (e.g., for mandibular variants), etc.) that may be user-specific (e.g., via digital scan and/or analog impression of a specific patient) or standardized (e.g., single size or various standard sizes (e.g., small, medium, large)), any suitable bonding interface 113, any suitable flexible interconnect 114, any suitable encapsulation component or encapsulation layer 115 (e.g., any suitable overmold 1020 or portion thereof, biocompatible material, elastomeric or polymeric material (e.g., for providing moisture resistance, electrical insulation, mechanical buffering, etc. (e.g., battery 510 may be mechanically supported and encapsulated within carrier structure 110 using encapsulation component 115))), any suitable biocompatible sealing component or biocompatible sealing layer 116 (e.g., any suitable overmold 1020 or portion thereof, biocompatible material, elastomeric or polymeric material (e.g., for providing moisture resistance, electrical insulation, mechanical buffering, etc. (e.g., battery 510 may be mechanically supported and encapsulated within carrier structure 110 using biocompatible sealing component 116))), any suitable module recess(es) or cavity(ies) 117 (e.g., any suitable placeholders or cavities or recesses (e.g., that may be any suitable computer-aided design (“CAD”)-defined, pre-defined, etc.), any suitable removable, common, standardized electronics modules, etc.), any suitable structural support element 118, and/or the like. Carrier structure 110 and intraoral shell 111/112 of FIG. 1C and FIG. 2 may correspond to shell 102s of FIG. 1B. Accordingly, shell 102s may represent a structural embodiment of carrier structure 110 in an assembled and/or encapsulated form. In some embodiments, the system may include a single-shell configuration that may be configured for either the upper jaw or the lower jaw. In other embodiments, separate shells may be provided for both jaws. A combined dual-jaw shell may also be provided. However, single-jaw configurations may be able to provide improved comfort, reduced occlusal interference, simplified insertion, and/or reduced mechanical loading during mastication (e.g. as compared to other possible configurations). Bonding interface 113 may include any suitable mechanical and/or chemical interface that may be configured to secure components within carrier structure 110, including, but not limited to, adhesive bonding regions, mechanical retention features, overmold anchoring surfaces, interfacial coupling structures between modules and the carrier, and/or the like. Flexible interconnect 114 may include one or more suitable flexible electrical interconnections, including, but not limited to, one or more flex-PCB traces, conductive films, wire-based connections, and/or the like that may be configured to electrically couple distributed components of subsystem 100 while accommodating anatomical curvature and deformation of the intraoral structure. Encapsulation layer 115 and biocompatible sealing layer 116 may correspond to overmold 1020 of FIG. 1B. In some embodiments, layer 115 and/or layer 116 may represent distinct functional sublayers of overmold 1020, including a structural encapsulation layer and a biocompatible sealing interface layer. Element 117 may represent any suitable CAD-defined module cavities, recesses, placeholders, and/or the like that may be configured to receive standardized electronics modules or any other suitable electronics modules. Accordingly, element 117 may refer to the structural interface (e.g., cavity), while the removable electronics modules (e.g., standardized modules) may be distinct components that may be configured to be received within such cavities. Structural support element 118 may include one or more reinforcement structures that may be configured to provide mechanical stability, load distribution, and/or deformation resistance of carrier structure 110, including, but not limited to, ribs, frames, embedded stiffeners, localized reinforcement regions, and/or the like. Element(s) 111-118 may be associated with carrier structure 110, as FIG. 1C may represent a functional system architecture and FIG. 2 may represent a spatial embodiment. Accordingly, these elements may be considered structural and functional components integrated within or supported by carrier structure 110.

It is to be understood that any suitable electronic components (e.g., one, some, or each electronic component of one, some, or each domain (e.g., one, some, or each of domains 300, 500, and 900)) may alternatively be housed in an extraoral unit that may be communicatively coupled (e.g., via any suitable wired or wireless connection(s)) to the intraoral module. One or more functional domains (e.g., processing, power management, communication, etc.) may be located outside the oral cavity. External device 1000 may represent such an extraoral unit. Carrier structure 110 may represent the intraoral unit. In some embodiments, sensing components may remain intraoral, while processing, power, and/or communication components may be partially or fully located extraorally and communicatively coupled to the intraoral structure.

FIG. 2 is an occlusal view of an upper jaw UUJ of a user wearing subsystem 100 (e.g., illustrating an exemplary intraoral placement of an intraoral apparatus of subsystem 100 within an upper jaw). As shown, the upper jaw UUJ may extend from a posterior (e.g., molars) side to an anterior (e.g., front teeth) side (e.g., in the +Y direction) and from a left buccal side to a right buccal side (e.g., in the +X direction), and may include any suitable arrangement of teeth, such as left central incisor LCI, left lateral incisor LLI, left canine LC, left first premolar LPM1, left second premolar LPM2, left first molar LM1, left second molar LM2, left third molar LM3, right central incisor RCI, right lateral incisor RLI, right canine RC, right first premolar RPM1, right second premolar RPM2, right first molar RM1, right second molar RM2, and right third molar RM3. FIG. 2 may represent an occlusal view of upper jaw UUJ, illustrating the occlusal surfaces (i.e., the biting surfaces) of the teeth. The terms “palatinal” and/or “lingual” may refer to the inner side of the dental arch facing the palate (e.g., upper jaw) or tongue (e.g., lower jaw), in contrast to the buccal side facing the cheeks. However, it is to be understood that any other suitable arrangement of teeth may be provided by a jaw wearing subsystem 100. FIG. 2 shows an illustrative relative positioning of various components of subsystem 100 (e.g., sensors 210, 211, 221, 222, 230, 231, and 240, electrodes 411, 412, 413L, 413R, 414L, and 414R, actuators 420 and 421, LEDs 431, 432, and 433, battery 510/511, charging coil 611, and at least a portion of an outer periphery boundary 110b of carrier structure 110) relative to the upper jaw UUJ. However, it is to be understood that a similar arrangement may be enabled for placement relative to an opposing lower jaw (e.g., for a mandibular arrangement).

Components of subsystem 100 may be positioned within carrier structure 110 such that they may be located between the occlusal plane of the teeth and the soft tissue surfaces when worn by the user. Certain components, such as pressure sensors 210/211, may be positioned near or slightly below the occlusal contact plane to detect force transmission, while other components may be embedded within or below the structural thickness of carrier structure 110. Thus, component positioning along the Z-axis may vary depending on function, such as with sensing elements positioned closer to interaction zones and electronics embedded deeper within the structure.

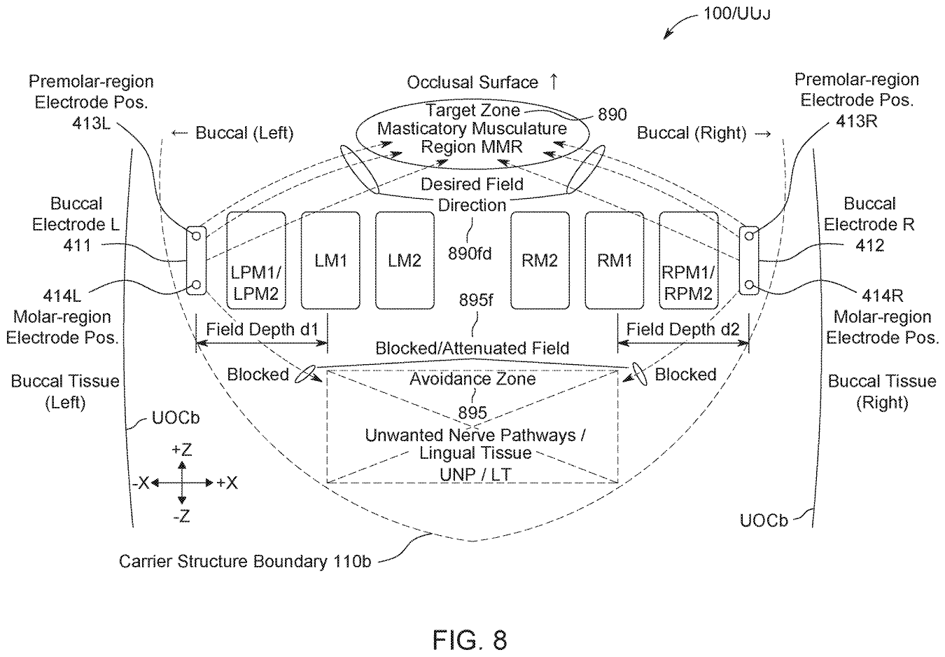

Carrier structure boundary 110b may be defined based on anatomical geometry derived from digital scans, standard dental arch models, and/or the like. The shape and extent of boundary 110b may be optimized to ensure mechanical retention and stability, minimize interference with occlusion and speech, position sensors and actuators at functionally relevant anatomical locations, distribute mechanical loads across the dental arch, and/or the like. An anatomical boundary UOCb of the oral cavity of the user may be shown (see, e.g., FIG. 2 and/or FIG. 8). Boundary UOCb may be a schematic outline of the dental arch and surrounding soft tissue regions (e.g., buccal, gingival, and vestibular boundaries). Boundary UOCb may be provided for spatial reference only, to illustrate the relative positioning of boundary 110b of intraoral carrier structure 110 with respect to the user's anatomical structures. Carrier structure boundary 110b may correspond to the physical extent of the intraoral device, whereas boundary UOCb may represent surrounding anatomical context.

The anterior and posterior extent of boundary 110b may be selected to balance retention and comfort, such as for avoiding excessive extension into anterior regions while ensuring stable anchoring in premolar and molar regions where mechanical support may be highest.

Occlusal pressure sensors 210/211 may be positioned in molar regions where peak occlusal forces may occur, thereby enabling detection of high-load events, such as bruxism or impact-related clenching. Their positioning near the outer boundary may allow efficient force transmission detection while maintaining structural support within carrier structure 110.

PPG sensors 221/222 may be positioned along inner (e.g., palatal/lingual) regions, such as to maximize contact with vascularized mucosal tissue, thereby improving signal quality for physiological measurements, such as pulse or perfusion-related signals.

Vibration actuators 420/421 may be positioned in posterior regions to maximize perceptibility and coupling to the dental arch, even in cases where third molars are absent, as stimulation may be transmitted through adjacent structures.

Premolar electrodes (e.g., electrodes 413L and 413R) may be positioned within the boundary of carrier structure 110 to define intermediate stimulation regions and contribute to shaping of the stimulation field along the dental arch.

Molar electrodes (e.g., electrodes 414L and 414R) may be positioned in posterior regions to define the longitudinal extent of the stimulation field and enable deeper field penetration toward target masticatory structures, while partial extension toward the boundary may improve field shaping and directional control.

Buccal electrodes 411/412 may be positioned along the buccal side of the dental arch, typically in proximity to molar or premolar regions, and within or near outer boundary 110b of carrier structure 110. Such positioning may be selected to maximize proximity to masticatory muscle insertion regions, enable effective current coupling through buccal tissue, provide directional field shaping toward target zones, minimize stimulation of lingual or undesired neural pathways, and/or the like. The positioning may be slightly inward from outer boundary 110b to maintain mechanical protection and structural integration while preserving effective tissue contact.

LEDs 431-433 may not always be positioned fully beyond boundary 110b. In some embodiments, LEDs may be partially embedded within carrier structure 110 and coupled to light-guiding elements or transparent/translucent regions of the encapsulation. This may allow optical output to be visible externally while maintaining structural integrity and sealing. The positioning may be selected to enable visibility to external observers, maintain mechanical robustness and sealing, avoid interference with occlusion and soft tissue, allow controlled light emission through defined optical windows, and/or the like.

Any suitable flexible interconnect(s) 114 may be provided to couple (e.g., communicatively, electrically, etc.) various other component(s) of subsystem 100. Flexible interconnect 114 may electrically couple sensors, actuators, electrodes, LEDs, power components, processing elements, and/or the like. Connections may include, but are not limited to, sensor-to-processing connections, processing-to-actuation connections, power distribution lines, stimulation signal routing to electrodes, communication lines between modules, and/or the like. Additional interconnects may extend from electrodes 413/414 and LEDs 431-433 to central processing and stimulation circuitry that may be located within carrier structure 110. Such interconnects may be implemented as flex-PCB traces, embedded conductive pathways routed within the carrier geometry, and/or the like.

LED 433 in FIG. 2 may represent a visible front-facing indicator, whereas LED driver 433 in FIG. 1C may represent the electronic driving circuitry. These may correspond to distinct functional elements, or alternatively may be represented using a shared reference number depending on context.

Carrier structure 110 may be represented in FIG. 2 by boundary 110b. In some embodiments, additional images (e.g., 3D views (e.g., similar to FIG. 1B)) may explicitly illustrate the full geometry of carrier structure 110 for clarity.

Upper jaw shell 111 may be represented in FIG. 2 as a structural body corresponding to carrier structure 110. In some embodiments, shell 111 may be explicitly illustrated and labeled in FIG. 2 or in additional images (e.g., 3D views (e.g., similar to FIG. 1B)).

In some embodiments, substantially all intraoral components of subsystem 100 may be integrated within shell 111. However, certain components, such as electrode contact surfaces, optical output regions, and/or the like, may be partially exposed to enable functional interaction with tissue or external visibility.

Bonding interface 113 may be represented as internal structural regions and may not be explicitly visible in FIG. 2. In some embodiments, it may be illustrated in sectional or exploded views.

Encapsulation layer 115 may correspond to an internal or intermediate layer and may not be explicitly visible in FIG. 2. It may be shown in cross-sectional or 3D representations (e.g., additional images (e.g., 3D views (e.g., similar to FIG. 1B)))

Biocompatible sealing layer 116 may form part of the outer surface and may be implicitly represented in FIG. 2. Explicit illustration may be provided in sectional or detailed figures.

Cavities 117 may be internal features and are not necessarily visible in FIG. 2. They may be illustrated in exploded or cross-sectional views.

Structural support element 118 may be integrated within carrier structure 110 and may not be explicitly visible in FIG. 2. It may be illustrated in structural or sectional diagrams.

Additional sensing elements, such as module 220, gyroscope 232, interface electronics 250, and/or the like, may be included but are not necessarily shown in FIG. 2 for clarity. These components may be embedded within the structure and may be represented in schematic FIG. 1C rather than spatial FIG. 2.

Components of processing domain 300 are not shown in FIG. 2, as FIG. 2 focuses on spatial placement of external-facing and/or anatomically relevant components. Processing components may be embedded within carrier structure 110.

Electrical stimulation subsystem 410 and visual signal indicator 430 may be functionally present but not explicitly shown in FIG. 2. Only their physical interfaces (e.g., electrodes, LEDs, etc.) may be illustrated in FIG. 2.

Beyond battery 510/511, additional power components are not shown in FIG. 2 to avoid clutter. They may be embedded within carrier structure 110.

Charging components (e.g., coil 611) may be selectively shown in FIG. 2. Other elements of subsystem 600 may be embedded and not explicitly illustrated in FIG. 2.

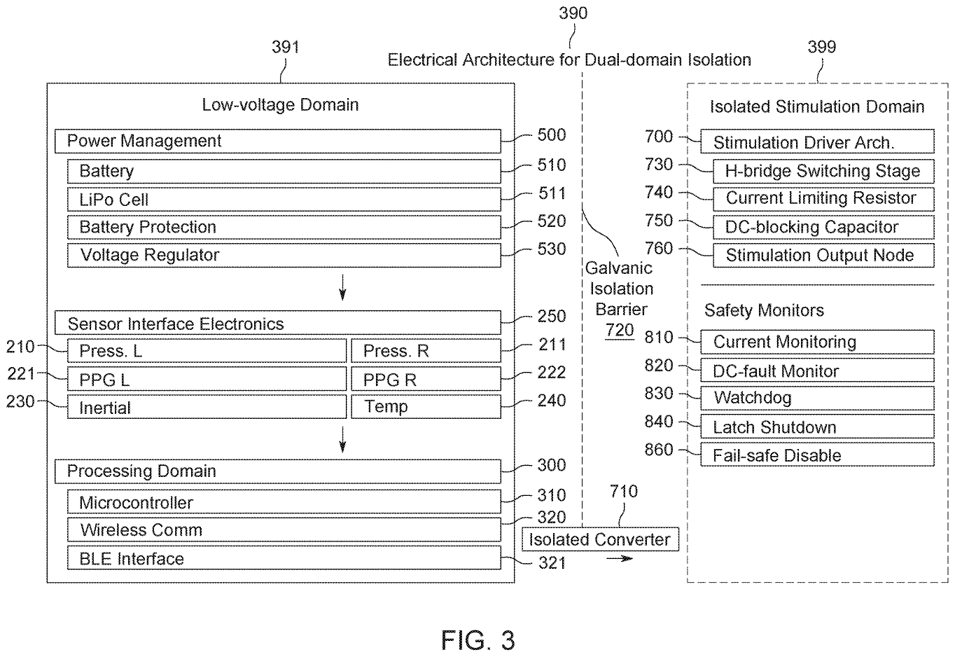

Stimulation driver architecture 700 is not shown in FIG. 2, as it represents internal circuitry rather than spatially relevant external elements. It is illustrated schematically in FIG. 1C and FIG. 3.

Safety domain 800 is not shown in FIG. 2 because it represents internal hardware safety circuitry. Such components may be embedded and not spatially exposed.

Escalation control logic 900 is not shown in FIG. 2, as it represents functional control logic rather than spatially relevant structure. It is represented schematically in FIG. 1C.

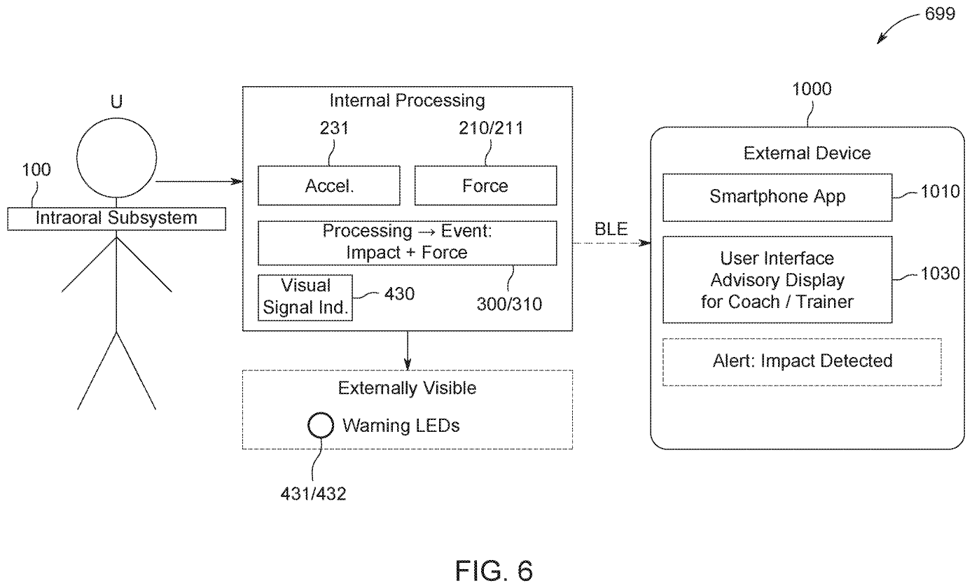

External device 1000 is generally not shown in FIG. 2, because FIG. 2 is intended to illustrate the intraoral spatial arrangement of subsystem 100 relative to the dental arch. External device 1000 is extraoral and functionally connected, but not anatomically positioned within the upper jaw arrangement shown in FIG. 2. External device 1000 may be illustrated in a separate system-level diagram, such as FIG. 1 and/or FIG. 6, where communication links between the intraoral subsystem and the external device may be viewed more clearly.

FIG. 3 illustrates an exemplary electrical architecture 390 for dual-domain isolation between any suitable domains, such as a low-voltage domain 391 and an isolated stimulation domain 399, using any suitable isolation barrier, such as galvanic isolation barrier 720 and/or isolated converter 710. The solid line of box 391 may indicate a physically implemented low-voltage domain, whereas the broken line of box 399 may indicate a functionally isolated stimulation domain whose exact physical implementation may vary. The broken line may further emphasize that the isolated stimulation domain may be defined by electrical isolation and safety partitioning rather than solely by physical enclosure. The horizontal arrow at converter 710 indicates transfer of energy across the isolation boundary from low-voltage domain 391 to isolated stimulation domain 399. Converter 710 may provide isolated power transfer between domains, whereas barrier 720 may provide galvanic isolation for signaling and control communication. Thus, converter 710 may enable energy transfer into isolated stimulation domain 399, while barrier 720 may prevent direct conductive coupling and/or may allow only isolated command, timing, and/or status transfer. As shown, low-voltage domain 391 may include any suitable component(s) of power management domain 500 (e.g., battery 510, LiPo cell 511, battery protection 520, voltage regulator 530, and/or the like), any suitable sensor interface electronics 250 and/or otherwise of sensing domain 200 (e.g., pressure sensors 210 and 211, PPG sensors 221 and 222, inertial sensing module 230, temperature sensor 240, and/or the like), any suitable component(s) of processing domain 300 (e.g., microcontroller 310, wireless communication interface 320, BLE interface 321, and/or the like), and/or the like. Certain components may be omitted from FIG. 3 for schematic clarity. FIG. 3 is intended to illustrate the principal isolation concept rather than an exhaustive circuit diagram. Accordingly, only representative components may be shown. The arrow between domains 500 and 200 may indicate power delivery from power management domain 500 to sensing components of sensing domain 200. The arrow between domains 200 and 300 may indicate signal flow from sensing domain 200 to processing domain 300 for evaluation and event classification. As shown, isolated stimulation domain 399 may include any suitable component(s) of stimulation driver architecture 700 (e.g., H-bridge switching stage 730, current limiting resistor 740, DC-blocking capacitor 750, stimulation output node 760, and/or the like), any suitable component(s) of safety domain 800 (e.g., any suitable safety monitor(s), current monitoring 810, DC-fault monitor 820, watchdog 830, latch shutdown 840, fail-safe disable 860, and/or the like), and/or the like. Component 731 may be omitted as an individual element in FIG. 3 because it may be understood as part of H-bridge switching stage 730. Thermal protection 850 may be omitted from FIG. 3 because FIG. 3 focuses primarily on stimulation-domain electrical isolation rather than battery thermal supervision. The horizontal line between components 700 and 800 may indicate supervisory interaction, whereby safety domain 800 monitors and can override operation of stimulation driver architecture 700. Microcontroller 310 may be configured to provide control signals only, and domain 399 may be galvanically isolated. In some embodiments, intraoral neuromodulation stimulation subsystem 410 may be considered part of isolated stimulation domain 399, as the stimulation pulses may be generated and delivered within the patient-side isolated domain. The stimulation request may be generated in the low-voltage processing domain and transferred across barrier 720 as an isolated control signal, timing signal, encoded command, heartbeat-supervised request, and/or the like. The isolated domain may then locally generate stimulation output subject to hardware-enforced safety limits. Galvanic isolation barrier 720 may be configured to protect the patient-side domain (e.g., isolated stimulation domain 399), which may include the stimulation output path and electrodes that may interface with intraoral tissue. In some embodiments, stimulation driver architecture 700 and at least part of safety domain 800 may be intraoral so as to remain within the isolated patient-side domain close to the electrodes. However, in alternative embodiments, portions of the circuitry may be extraoral, provided that patient-contacting stimulation outputs remain galvanically isolated. Sensing domain 200 is preferably intraoral because it may detect occlusal, physiological, and motion-related conditions associated with the oral cavity.

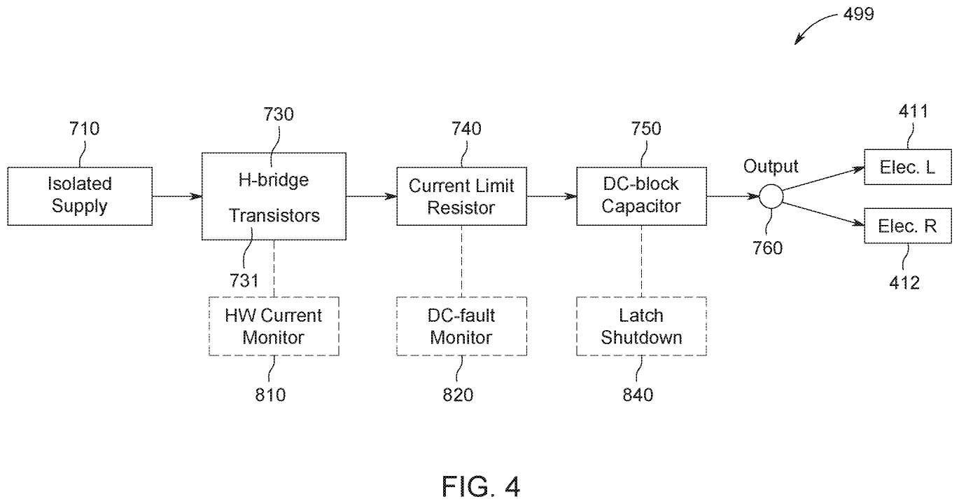

FIG. 4 illustrates an exemplary biphasic electrical stimulation path 499 that may include isolated supply 710 and then an H-bridge switching stage 730 (e.g., with any suitable switching transistor(s) 731), which may be monitored by hardware current monitor 810, and then a current limit resistor 740, which may be monitored by DC-fault monitor 820, and then a DC-block capacitor 750, which may be monitored by latch shutdown circuit 840, and then a stimulation output node 760 and then electrode(s) (e.g., left buccal electrode 411 and right buccal electrode 412). The dashed lines in FIG. 4 may indicate functional supervision relationships rather than direct current-carrying conduction paths. Overall, FIG. 4 may illustrate a safe biphasic stimulation chain in which isolated power may be converted into a controlled pulse output, monitored for overcurrent and DC-fault conditions, and coupled to tissue through a DC-blocking architecture. This may provide unique combination of galvanically isolated stimulation generation, biphasic charge-balanced delivery, independent hardware current monitoring, DC-fault monitoring across a blocking capacitor, and latch-based fail-safe shutdown. This architecture may provide a hardware-enforced patient protection chain that may be particularly suited for intraoral neuromodulation.

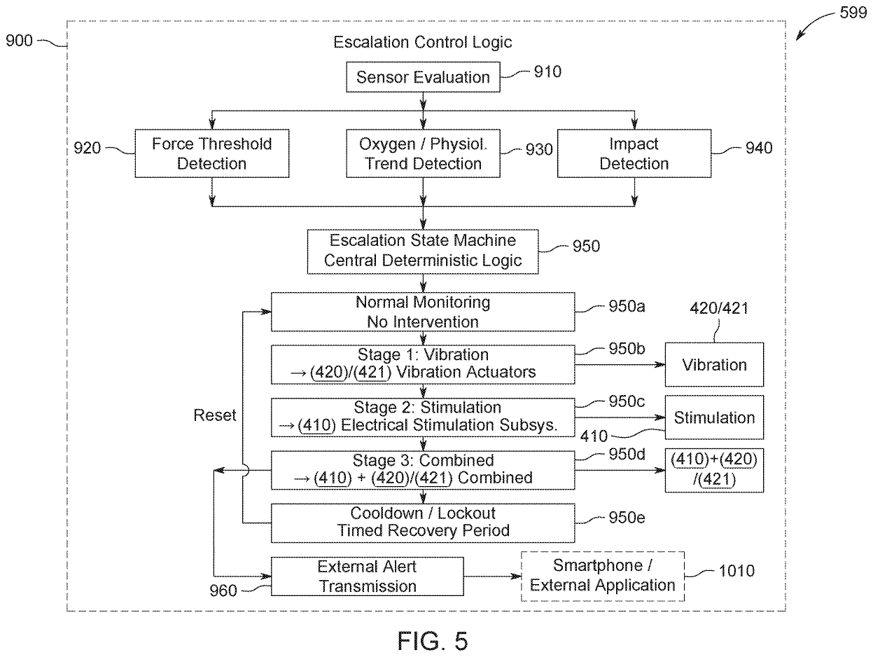

FIG. 5 illustrates an exemplary functional flow or process 599 for staged escalation based on detected any suitable mechanical and/or physiological parameters using escalation control logic 900. As shown in FIG. 5, escalation control logic 900 may endeavor process 599 by including any suitable sensor evaluation operation 910 that may lead, in parallel, to each one of a force threshold detection operation 920, an oxygen/physiological trend detection operation 930, and an impact detection operation 940. Next, process 599 may include using any suitable escalation state machine 950 (e.g., central deterministic logic), which might include any suitable normal monitoring operation 950a (e.g., no intervention), and then any suitable stage 1 vibration operation 950b (e.g., for actuating any suitable vibration (e.g., with any suitable vibration actuator(s) 420/421)), and then any suitable stage 2 stimulation operation 950 c (e.g., for actuating any suitable stimulation (e.g., with any suitable electrical stimulation subsystem 410)), and then any suitable stage 3 combined operation 950d (e.g., for actuating any suitable vibration and stimulation (e.g., with any suitable vibration actuator(s) 420/421 and any suitable electrical stimulation subsystem 410)), and then any suitable cooldown/lockout operation 950e (e.g., for enduring any suitable timed recovery period), and then resetting by returning to operation 950a. During cooldown/lockout operation 950e, stimulation may be inhibited for a predefined or adaptive time period, vibration may be reduced or stopped, event accumulation may continue to be monitored, and/or escalation to a higher stage may be temporarily blocked. The cooldown duration may be fixed, timer-based, dose-based, or adaptive based on cumulative exposure, severity, or reduction in event activity. During reset return to operation 950a, the system may clear temporary escalation states, maintain safety supervision, preserve historical event data, and/or resume baseline monitoring without active intervention. In some embodiments, after operation 950d, process 599 may also proceed to any suitable external alert transmission operation 960 (e.g., for carrying out any suitable control on any suitable smartphone/external application 1010 (e.g., of external device 1000)). Operation 960 may include transmission of an alert, advisory state, event summary, severity level, threshold exceedance notification, and/or the like to external device 1000. An advisory device state may correspond to a condition in which predefined thresholds are exceeded and may trigger non-diagnostic alerts, recommendations, or notifications via an external application. In some embodiments, such external alerting may occur after operation 950d, as stage 3 combined intervention may represent a sufficiently persistent or severe event to justify external reporting. In other embodiments, alert transmission may occur at earlier stages depending on configuration. Process 599 may involve any suitable deterministic state transitions, process 599 may not involve any AI-based or adaptive decision logic, and/or all thresholds of process 599 may be firmware-configured. This may provide various unique aspects, including, but not limited to, combined evaluation of occlusal force and inertial motion, staged escalation across multiple intervention modalities, integration of physiological trend inputs, cooldown/lockout behavior linked to cumulative exposure or dose, safety-constrained intervention logic in a closed-loop intraoral system, and/or the like.

It is understood that the operations shown in process 599 of FIG. 5 are only illustrative and that existing operations may be modified or omitted, additional operations may be added, and the order of certain operations may be altered.

FIG. 6 illustrates a scenario 699 for impact and contact-sport monitoring of an athlete user U that may be using any suitable intraoral subsystem 100, where any suitable impact event experienced by the user may lead to any suitable internal classification and external warning (e.g., external visible LED warning on an intraoral apparatus) and external app notification. For example, any suitable internal processing may be carried out by any suitable portion(s) of sensing domain 200, P&C domain 300, and/or actuation domain 400 (e.g., any suitable combination of acceleration sensing 231, occlusal pressure or force sensing 210 and/or 211, any suitable event processing with microcontroller unit 310 based on any suitable sensing domain signal(s) (e.g., impact and/or force)) for determining any suitable actionable event. In some embodiments, event detection may be based on a multi-operation processing flow that may include (1) acquisition of occlusal force data and inertial motion data, (2) threshold evaluation for each modality, (3) temporal correlation of threshold exceedances, (4) consistency evaluation across multiple detection cycles, (5) suppression of event classification when conflicting or non-correlated signals are detected, and (6) assignment of a severity level or protective device state. This processing flow may be used to distinguish impact-related events from normal chewing, speech, or isolated sensor artifacts. Such internal processing may also include any suitable visual signal indicator(s) 430 that may be triggered by microcontroller unit 310 in response to such event detection (e.g., for adjusting any suitable externally visible features of intraoral subsystem 100 (e.g., LED(s) 431, 432, 433, etc.)) for generating any suitable local visual warning(s) to a user. Visual signal indicator 430 may refer to a functional signaling subsystem, whereas LEDs 431-433 may refer to specific physical light-emitting elements that may implement such signaling. In some embodiments, local warning may additionally or alternatively be provided by any suitable audible indicator(s), acoustic transducer(s), haptic actuator(s), and/or any suitable combination(s) thereof, which may be integrated into carrier structure 110 or otherwise coupled thereto. Additionally or alternatively, such internal processing may be configured to trigger any control (e.g., via BLE) by microcontroller unit 310 in response to such event detection (e.g., for adjusting any suitable component(s) of external device 1000 (e.g., any suitable smartphone app 1010, any suitable user interface 1030 (e.g., advisory display for any suitable coach/trainer of the user), any suitable alert (e.g., impact event detected) may be presented by external device 1000 (e.g., any suitable output component(s) 160), and/or the like (e.g., for enabling any suitable third-party intervention).

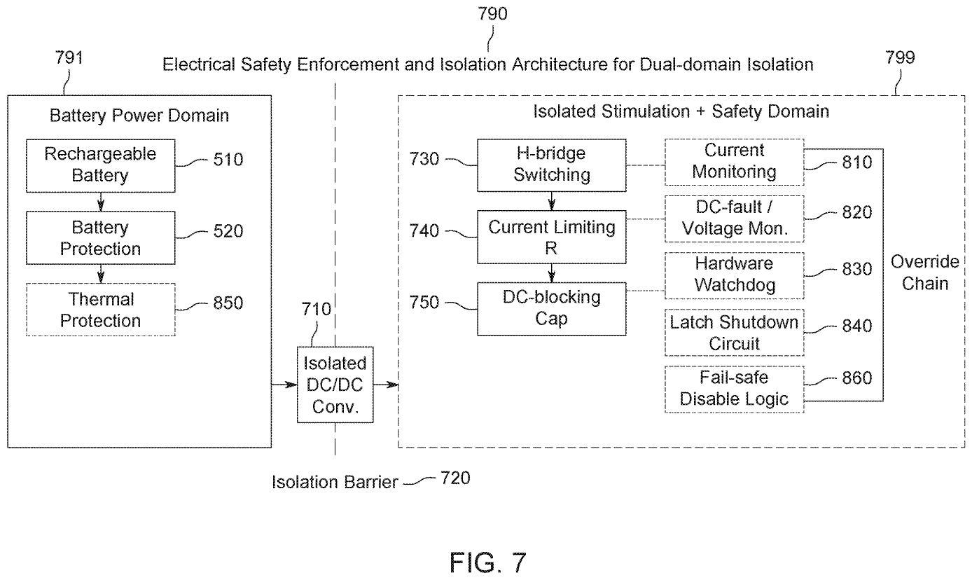

FIG. 7 illustrates an exemplary electrical safety enforcement and isolation architecture 790 for dual-domain isolation between any suitable domains, such as a battery/power domain 791 and an isolated stimulation and safety domain 799, using any suitable isolation barrier, such as isolation barrier 720 and/or isolated DC/DC converter 710. The solid line of box 791 may represent a conventional low-voltage battery/power domain, whereas the broken line of box 799 may indicate a functionally isolated patient-side stimulation and safety domain whose implementation may vary but may remain electrically separated. Converter 710 may be configured to transfer power across the isolation boundary, while barrier 720 may be configured to provide signal isolation and protect against direct conductive coupling between domains. As shown, battery/power domain 791 may include any suitable component(s) of power management domain 500 (e.g., rechargeable battery 510, battery protection 520, and/or the like), any suitable component(s) of safety domain 800 (e.g., thermal protection 850), and/or the like. Components may be omitted from FIG. 7 for schematic clarity, such that only representative safety-relevant elements may be illustrated. Thermal protection 850 may be shown in domain 791 because battery-related thermal monitoring may be closely associated with the battery/power domain. As shown, isolated stimulation and safety domain 799 may include any suitable component(s) of stimulation driver architecture 700 (e.g., H-bridge switching stage 730, current limiting resistor 740, DC-blocking capacitor 750, and/or the like), any suitable component(s) of safety domain 800 (e.g., any suitable current monitoring 810, DC-fault/voltage monitor 820, hardware watchdog 830, latch shutdown circuit 840, fail-safe disable 860, and/or the like (e.g., as an override chain (e.g., a safety supervision path in which any one of the safety monitors may trigger a disabling condition that may override a software stimulation request and force shutdown through latch 840)), and/or the like. Arrows between components may indicate power flow or signal flow within the stimulation chain. Horizontal lines from stimulation path elements to safety monitors may indicate supervisory monitoring relationships rather than primary current flow. Galvanic isolation barrier 720 may be configured to protect the patient-side domain (e.g., isolated stimulation and safety domain 799), which may contain a stimulation output path and safety elements associated with patient-contacting stimulation delivery. In some embodiments, stimulation driver architecture 700 and safety domain 800 may be intraoral. In other embodiments, portions may be externalized, provided patient-contacting stimulation remains within an isolated, safety-supervised domain. Battery domain 500 may be intraoral or extraoral depending on implementation.

FIG. 3 may primarily illustrate general dual-domain electrical separation between low-voltage electronics and an isolated stimulation domain, while FIG. 7 may emphasize safety enforcement within the isolated stimulation path, including the safety override chain and the relationship between the stimulation driver path and the safety monitors. Therefore, FIG. 3 may focus on architectural isolation, whereas FIG. 7 may focus on safety enforcement architecture.

FIG. 8 is a buccal view of upper jaw UUJ of a user wearing subsystem 100 (e.g., illustrating an exemplary intraoral placement of an intraoral apparatus of subsystem 100 within an upper jaw). As shown, the upper jaw UUJ may have a masticatory musculature region MMR that may be an objective of a target zone 890 of subsystem 100, and unwanted nerve pathways UNP and/or lingual tissue LT that may be an objective of an avoidance zone 895 of subsystem 100, and may extend from left buccal tissue to right buccal tissue (e.g., in the +X direction), and may include any suitable arrangement of teeth, such as left central incisor LCI, left lateral incisor LLI, left canine LC, left first premolar LPM1, left second premolar LPM2, left first molar LM1, left second molar LM2, left third molar LM3, right central incisor RCI, right lateral incisor RLI, right canine RC, right first premolar RPM1, right second premolar RPM2, right first molar RM1, right second molar RM2, and right third molar RM3 (e.g., FIG. 2 may show the bottoms of these teeth of upper jaw UUJ, while FIG. 8 may show the outer surfaces of these teeth facing respective left and right buccal tissue (e.g., a cross-section coronal plane)). However, it is to be understood that any other suitable arrangement of teeth may be provided by a jaw wearing subsystem 100, and a similar arrangement of subsystem 100 may be enabled for placement relative to an opposing lower jaw (e.g., for a mandibular arrangement (e.g., geometry may be equally applicable to mandibular (lower jaw) placement with mirrored electrode positions)). While FIG. 2 may be understood as an occlusal/top view of upper jaw UUJ, FIG. 8 may be understood as a buccal-side view or sectional side view showing the relationship of electrodes to buccal tissue and adjacent anatomical target zones of upper jaw UUJ, where the X-axis may extend left-right across the buccal surfaces, the Y-axis may extend posterior-anterior along the dental arch, and the Z-axis may extend inward-outward relative to the tissue and electrode depth. FIG. 8 shows an illustrative relative positioning of various components of subsystem 100 (e.g., electrodes 411, 412, 413, and 414, and at least a portion of an outer periphery boundary 110b of carrier structure 110) relative to the upper jaw UUJ. FIG. 8 shows selective stimulation geometry and anatomical targeting, such as buccal electrode placement for electrodes 411, 412, 413, and 414, which may be configured to affect preferentially any suitable target region(s) (e.g., target zone 890) with any suitable desired field direction 890fd and/or to minimize field spread toward undesired nerve pathways UNP (e.g., avoidance zone 895) with any blocked/attenuated field(s) 895f, which may have any suitable field depth(s) (e.g., field depth d1 and/or field depth d2). Targeting may be achieved by the relative positioning, spacing, polarity selection, and geometric extent of electrodes 411, 413, and 414, such that current density may be directed toward the intended buccal-side target region while limiting deeper or lingual spread. Avoidance may be achieved by constraining the stimulation field geometry, selecting electrode spacing, controlling current amplitude, and positioning electrodes to reduce field extension toward undesired nerve pathways or lingual tissue. Field depths d1 and d2 may illustrate different penetration depths or field extents associated with different electrode configurations, amplitudes, or stimulation modes. These depths help teach that electrode geometry may be used to control how superficially or deeply stimulation is delivered. While electrodes 413R and 414R may be shown in FIG. 8 (e.g., if FIG. 8 is intended to illustrate bilateral symmetry or mirrored electrode placement), only the representative side may be shown for clarity if FIG. 8 is intended as a sectional or unilateral conceptual targeting view.

Extraoral sensors may generally not replace intraoral occlusal sensing for direct detection of tooth-loading events. However, extraoral sensors may supplement intraoral sensing (e.g., by detecting head acceleration, jaw motion, physiological state, and/or contextual information using any suitable wearable device(s) (e.g., ear-worn units, headbands, wrist-worn devices, etc.)). Thus, force/impact detection may remain intraoral or hybrid, while extraoral sensors provide supplemental data.

FIG. 2 and FIG. 8 may depict different perspectives and serve different explanatory purposes. FIG. 2 may emphasize spatial placement relative to the dental arch, whereas FIG. 8 may emphasize field shaping and anatomical targeting. Accordingly, the apparent overlap or enclosure of electrodes may be schematic and not intended to imply contradictory physical geometry.

Premolar and molar electrodes 413 and 414 may define the longitudinal extent of the stimulation field by setting spatial boundaries for current flow along the dental arch. By selecting which electrodes are active and how they are paired with buccal electrodes, the effective field can be lengthened, shortened, shifted, or focused. Therefore, intraoral electrode geometry may be used not merely for stimulation delivery, but for selective anatomical targeting and controlled field shaping within a safety-constrained neuromodulation platform.

Avoidance zone 895 may be achieved by selecting electrode geometry, spacing, relative polarity, and field orientation such that current density may be preferentially directed toward target zone 890 and attenuated toward lingual or unwanted nerve regions. This selective geometry may reduce unintended activation by limiting field spread beyond desired anatomical boundaries.

Boundary 110b may be designed based on anatomical scan data, comfort constraints, retention requirements, and/or target component placement. The boundary may be selected to retain the intraoral structure securely, provide sufficient space for sensors, electrodes, power, and interconnects, avoid unnecessary extension into sensitive or obstructive regions, support targeted stimulation geometry, and/or the like. If the extent shown in FIG. 2 runs from in front of premolar LPM1 to behind the canine region, FIG. 8 could be made consistent with that geometry to the extent practicable. An additional 3D perspective image might be provided to show the relationship between teeth, carrier structure 110, and boundary 110b in various detail. Converter 710 and isolation barrier 720 are not intended to be depicted as discrete macroscopic physical structures in FIGS. 1B, 2, and/or 8, as they may be implemented as integrated electrical components within the internal electronics architecture of the intraoral device. In some embodiments, isolated converter 710 and barrier 720 may be physically implemented within the internal electronics assembly (e.g., circuitry 102c) that may be embedded inside carrier structure 110 and/or intraoral shell (e.g., shell 102s). These components may be integrated at the interface between a low-voltage control and sensing region (e.g., sensing domain 200 and processing and communication domain 300) and an isolated stimulation region (e.g., stimulation driver architecture 700 and safety domain 800). Physically, this separation may be realized within a multilayer flexible PCB, rigid-flex PCB, miniaturized electronics module, and/or the like, such that isolation barrier 720 may be positioned electrically between conductive traces associated with the respective domains, rather than forming a visible structural boundary. The isolation boundary defined by barrier 720 may not necessarily correspond to a visible geometric boundary of carrier structure 110 or shell 102s, but instead may represent an internal electrical separation within the embedded electronics. In some embodiments, an isolated stimulation domain may be located in a posterior and/or lateral region of the intraoral device (e.g., proximate to electrodes 411, 412, 413, 414), while the sensing and processing domains may be located in a central and/or protected region (e.g., palatal or central electronics region), with isolation barrier 720 electrically separating these regions within the same physical carrier structure. In some embodiments, isolation barrier 720 may be implemented using transformer-based isolation, capacitive digital isolators, optocouplers, integrated isolated power modules, combinations thereof, and/or the like, and may be co-packaged within one or more integrated circuits or modules embedded within the intraoral device.

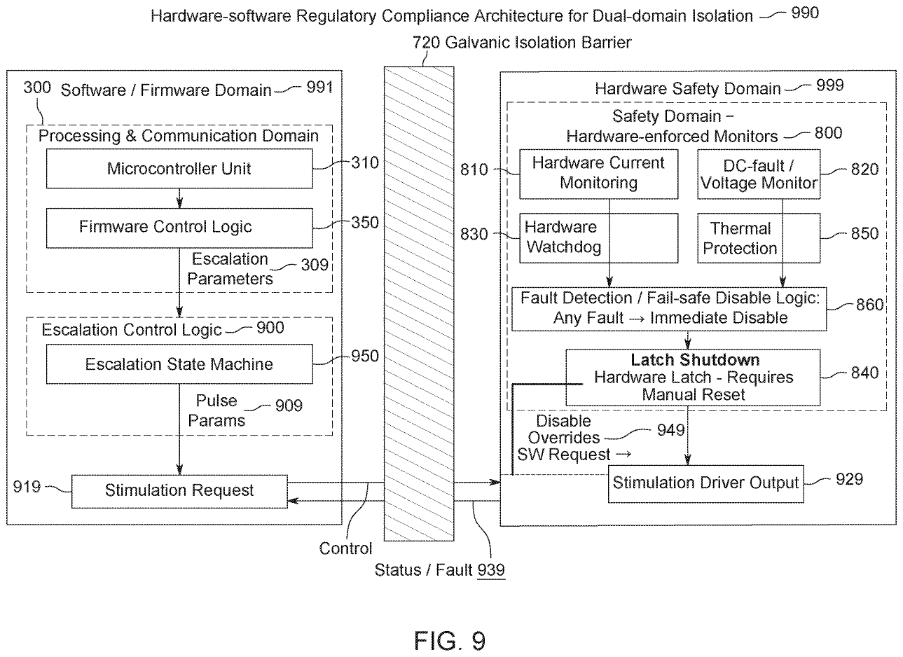

FIG. 9 illustrates an exemplary hardware-software regulatory compliance architecture 990 for dual-domain isolation between any suitable domains, such as a software/firmware domain 991 and an isolated hardware safety domain 999, using any suitable isolation barrier, such as galvanic isolation barrier 720. The solid outlines of boxes 991 and 999 may indicate the two principal regulatory and functional domains, whereas the broken-line references to domains 300, 900, and 800 may indicate that such previously introduced domains may be mapped into the higher-level compliance architecture 990 for explanatory purposes. Converter 710 may be omitted from FIG. 9 because FIG. 9 may primarily illustrate the logical and regulatory separation between software-controlled functions and hardware-enforced safety functions, rather than the power-transfer architecture shown in FIGS. 3 and 7. Therefore, FIG. 9 may illustrate the boundary between software control (e.g., of P&C domain 300, escalation control logic 900, etc.) and hardware-enforced limits (e.g., safety domain 800, etc.), such as by showing that, at least in some embodiments of the BMSP, firmware/app logic may not override hardware-enforced safety constraints. As shown, software/firmware domain 991 may include any suitable component(s) of P&C domain 300 (e.g., microcontroller unit 310, firmware control logic 350, and/or the like), any suitable component(s) of escalation control logic 900 (e.g., escalation state machine 950, etc.), and/or the like. The phrase “modifiable-subject to validation” may indicate that Parameters within software/firmware domain 991 may be configured, updated, or tuned, provided that such changes remain subject to predefined validation rules, safe parameter boundaries, and/or system acceptance criteria. Components 320, 321, 330, and 340 may be omitted from FIG. 9 for schematic clarity, because FIG. 9 may emphasize the compliance boundary between software-requested behavior and hardware-enforced safety rather than a detailed implementation of all processing-domain elements. The arrow between controller 310 and firmware control logic 350 may represent execution and control flow, whereby firmware logic may execute on or may be coordinated by microcontroller unit 310. Components 910, 920, 930, 940, and 960 may likewise be omitted from FIG. 9, because FIG. 9 may focus on representative escalation and compliance logic rather than all lower-level processing subfunctions. P&C domain 300 may be configured to provide any suitable escalation parameters 309 to escalation control logic 900. Escalation control logic 900 may utilize such escalation parameters 309 to provide any suitable pulse parameters 909. Escalation parameters 309 may include any suitable configuration variables used by escalation control logic 900, including, but not limited to, threshold values, timing windows, severity ranges, cooldown intervals, lockout rules, mode-selection criteria, and/or the like. Such parameters may be derived from device settings, user-specific calibration, historical sensor data, validated clinician-defined limits, and/or the like. Therefore, escalation parameters may be used in a safety-constrained, multi-modal intraoral neuromodulation architecture in which software-selected escalation behavior may remain bounded by independent hardware safety enforcement. A stimulation request 919 may be generated by domain 991 (e.g., based on pulse parameters 909) and may be sent as any suitable control to hardware safety domain 999. Stimulation request 919 may be generated by microcontroller unit 310 and/or escalation control logic 900 in domain 991 based on evaluated event conditions and selected pulse parameters 909. Stimulation request 919 may be communicated across barrier 720 as an isolated digital control signal, encoded timing signal, gated request, heartbeat-qualified enable signal, other isolated control mechanism, and/or the like. Within hardware safety domain 999, stimulation request 919 may be received by stimulation control circuitry, hardware gating circuitry, and/or other safety-qualified interface logic that may determine whether the request may be passed onward to the stimulation driver path. A purpose of stimulation request 919 may be to request stimulation delivery, but not to define or override the ultimate hardware-enforced safety limits of that delivery. Software or firmware may request stimulation, but hardware safety domain 999 may independently determine whether that request is permitted, modified, limited, or blocked according to non-overridable hardware safety constraints. As shown, hardware safety domain 999 may include any suitable component(s) (e.g., hardware-enforced monitors) of safety domain 800 (e.g., hardware current monitoring 810, DC-fault/voltage monitor 820, hardware watchdog 830, thermal protection 850, fault detection/fail-safe disable logic 860 (e.g., such that any fault detected may be immediately disabled), latch shutdown 840 (e.g., a hardware latch that may use a manual reset), etc.), and/or the like. Hardware safety domain 999 may operate autonomously from software execution and may continue to enforce safety constraints regardless of firmware state, application logic, communication state, or software commands. The arrows from current monitor 810 and DC-fault monitor 820 to fault detection/fail-safe disable logic 860 may represent supervisory fault propagation paths, whereby detection of any unsafe condition may contribute to a common disable decision. These arrows need not imply that current monitor 810 bypasses watchdog 830 or that DC-fault monitor 820 bypasses thermal protection 850; rather, they may indicate that multiple independent fault sources may converge on fail-safe disable logic 860. As shown, a stimulation driver output 929 may be provided for receiving any suitable output from latch shutdown 840 and may provide any suitable feedback 949 to latch shutdown 840 and any suitable status/fault 939 to domain 991. Stimulation driver output 929 may be produced by the stimulation driver path, such as H-bridge stage 730 and associated output circuitry, following permission by latch shutdown 840 or related hardware enable logic. Feedback 949 may include any suitable hardware status indicative of output activity, output-enable state, latched fault state, current-flow confirmation, and/or other driver-state feedback provided to latch shutdown 840 or associated safety logic for supervision. Any disable condition generated by safety domain 999 may take precedence over a stimulation request originating from software domain 991. Status/fault signal 939 may include fault flags, disable-state information, watchdog status, hardware-permitted state information, and/or the like. Status/fault signal 939 may be transferred across barrier 720 as an isolated signal to domain 991, where it may be received by microcontroller unit 310, firmware logic 350, and/or any suitable related monitoring software for logging, user notification, or adaptation of subsequent requests. The safety monitors of safety domain 800 may operate in hardware, independent of firmware. Hardware independence may be enabled by implementing monitoring, threshold comparison, and shutdown logic in dedicated hardware circuits, such as comparators, latches, watchdog timers, analog monitors, or discrete logic paths, rather than relying on firmware execution for fault enforcement. Fault detection/fail-safe disable logic 860 may be configured to trigger latch shutdown 840. Fault detection/fail-safe disable logic 860 may be directly coupled to latch shutdown 840 using hardwired fault paths such that any detected unsafe condition may force a latched disable state without requiring processor intervention. Galvanic isolation barrier 720 may be configured to protect the patient-side domain (e.g., hardware safety domain 999 and the associated patient-contacting stimulation path). Safety domain 800 may be preferably associated with the patient-side stimulation path, although portions of its implementation may vary by embodiment. Domains 300 and 900 may be intraoral or extraoral depending on implementation, whereas safety-critical patient-contacting stimulation outputs may remain associated with the isolated patient-side domain.

FIG. 3 may primarily illustrate electrical separation between a low-voltage sensing/processing domain and an isolated stimulation domain, while FIG. 7 may primarily illustrate safety enforcement within the isolated stimulation path, including the relationship between stimulation circuitry and the hardware safety override chain, while FIG. 9 may primarily illustrate the regulatory and functional boundary between software-controlled decision logic and hardware-enforced safety limits, emphasizing that software may request stimulation but cannot override the hardware safety domain.

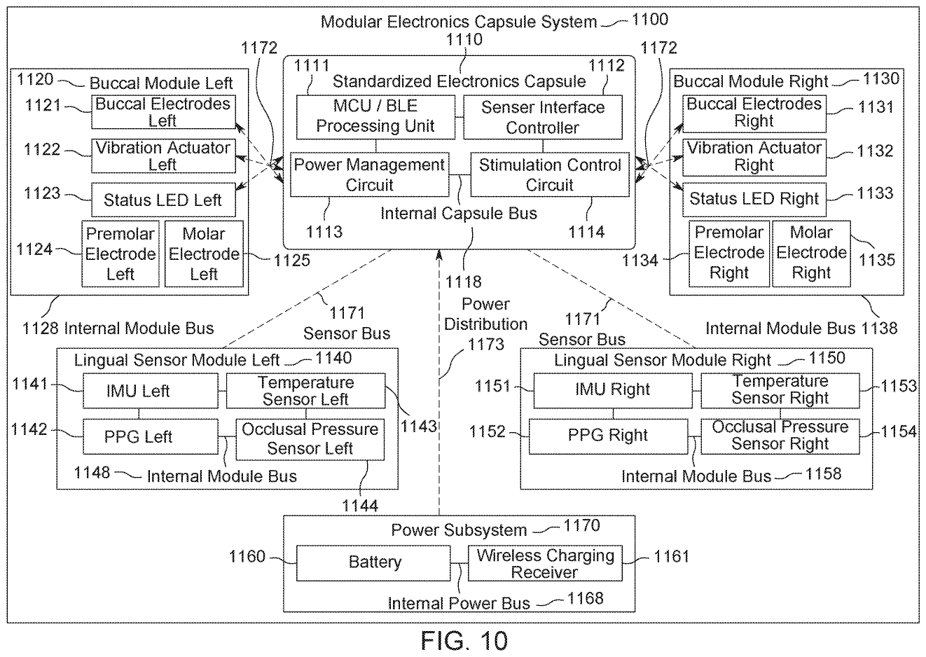

FIG. 10 illustrates a semi-exploded view of a modular electronics capsule system 1100, which may be a standardized, reusable, patient-independent electronics module. Modular electronics capsule system 1100 may correspond to one implementation of intraoral circuitry 102c of FIG. 1B, particularly in embodiments in which circuitry is packaged as a reusable, patient-independent module system. As shown, modular electronics capsule system 1100 may include a standardized electronics capsule 1110 and a power subsystem 1170 that may be configured to provide any suitable power to standardized electronics capsule 1110 via any suitable power distribution bus 1173. Electronics capsule 1110 may be reusable across patients and may be provided with any suitable design (e.g., a snap-in design). Standardized electronics capsule 1110 may be patient-independent. Standardized electronics capsule 1110 may be integrated into either a patient-specific shell (e.g., as may be generated from an impression or scan) or a standardized shell of one of multiple predetermined sizes, and such shells may be configured for either upper-jaw or lower-jaw use. Carrier structure 110 and/or shell 111 (e.g., shell 102s) may be represented in FIG. 10 as the receiving structure into which electronics capsule 1110 and associated modules may be installed. Encapsulation layer 115 and sealing layer 116 (e.g., overmold 1020) may be represented in FIG. 10 as enclosing or partially enclosing the assembled capsule system after integration into the shell. Circuitry 102c of FIG. 1B may correspond to electronics capsule 1110 and/or associated intraoral submodules shown in FIG. 10. System 1100 may be integrated into a shell geometry corresponding to FIG. 2 and/or FIG. 8 and/or into any suitable 3D exploded or assembled illustrations (see, e.g., FIG. 1B). Additionally or alternatively, power subsystem 1170 may be reusable across patients and may be provided with any suitable design. In some embodiments, some or all of electronics capsule 1110 and/or power subsystem 1170 may be housed intraorally within a patient during use of system 1100. However, in other embodiments, electronics capsule 1110 and power subsystem 1170 may be housed extraorally with any suitable wired and/or wireless coupling(s) to any intraoral components of system 1100 (e.g., electrodes). Electronics capsule 1110 may include any suitable components, including, but not limited to, a microcontroller unit (“MCU”)/communications (e.g., BLE) processing unit 1111 (see, e.g., component(s) 310, 320, and/or 321 of FIG. 1C), a sensor interface controller 1112 (see, e.g., component 340 of FIG. 1C), a power management circuit 1113 (see, e.g., component 520 of FIG. 1C), a stimulation control circuit 1114 (see, e.g., component 410 of FIG. 1C), and/or the like, all of which may be intercoupled by any suitable internal capsule bus 1118. Power subsystem 1170 may include any suitable components, including, but not limited to, a battery 1160 (see, e.g., component(s) 510 and/or 511 of FIG. 1C), a wireless charging receiver 1161 (see, e.g., component(s) 610 and/or 611 of FIG. 1C), and/or the like, all of which may be intercoupled by any suitable internal power bus 1168. Such references between FIG. 10 and FIG. 1C may be understood as functional correspondences rather than one-to-one structural identity. For example, MCU/communications processing unit 1111 may correspond functionally to components 310, 320, and 321, sensor interface controller 1112 may correspond functionally to component 340 and related sensing interface functions, power management circuit 1113 may correspond functionally to component 520 and related power conditioning, stimulation control circuit 1114 may correspond functionally to subsystem 410 and/or related stimulation-control circuitry, and/or the like. Likewise battery 1160 and wireless charging receiver 1161 may be understood as functional counterparts of components 510/511 and components 610/611, respectively.

As shown in FIG. 10, modular electronics capsule system 1100 may include one or more buccal modules, such as a left buccal module 1120 and a right buccal module 1130 (e.g., bilateral and/or peripheral buccal modules), component(s) of each of which may be functionally coupled to electronics capsule 1110 by any suitable stimulation signal bus(ses) 1172. Left buccal module 1120 may include any suitable components, including, but not limited to, one or more left buccal electrodes 1121 (see, e.g., component 411 of FIG. 1C), a left vibration actuator 1122 (see, e.g., component 420 of FIG. 1C), a left status LED 1123 (see, e.g., component 431 of FIG. 1C), a left premolar electrode 1124 (see, e.g., component 413L of FIG. 1C), a left molar electrode 1125 (see, e.g., component 414L of FIG. 1C), and/or the like, all of which may be intercoupled by any suitable internal module bus(ses) 1128. Right buccal module 1130 may include any suitable components, including, but not limited to, one or more right buccal electrodes 1131 (see, e.g., component 412 of FIG. 1C), a right vibration actuator 1132 (see, e.g., component 421 of FIG. 1C), a right status LED 1133 (see, e.g., component 432 of FIG. 1C), a right premolar electrode 1134 (see, e.g., component 413R of FIG. 1C), a right molar electrode 1135 (see, e.g., component 414R of FIG. 1C), and/or the like, all of which may be intercoupled by any suitable internal module bus(ses) 1138. In some embodiments, more than one left buccal electrode 1121 and/or more than one right buccal electrode 1131 may be provided (e.g., to enable field shaping, redundancy, selectable stimulation geometries, different electrode pairings along the buccal surface, etc.). Such buccal electrodes may be distributed along posterior buccal regions adjacent to premolar and molar zones and may be selectively addressable. Electrode(s) 1124, 1125, 1134, and/or 1135 may contribute to buccal-side field shaping and may be physically grouped with the corresponding side-specific stimulation module.

As shown in FIG. 10, modular electronics capsule system 1100 may include one or more lingual sensor modules, such as a left lingual sensor module 1140 and a right lingual sensor module 1150 (e.g., bilateral and/or peripheral lingual sensor modules), component(s) of each of which may be functionally coupled to electronics capsule 1110 by any suitable sensor bus(ses) 1171. Left lingual sensor module 1140 may include any suitable components, including, but not limited to, a left IMU 1141 (see, e.g., component 230 of FIG. 1C), a left PPG sensor 1142 (see, e.g., component 221 of FIG. 1C), a left temperature sensor 1143 (see, e.g., component 240 of FIG. 1C), a left occlusal pressure sensor 1144 (see, e.g., component 210 of FIG. 1C), and/or the like, all of which may be intercoupled by any suitable internal module bus(ses) 1148. Right lingual sensor module 1150 may include any suitable components, including, but not limited to, a right IMU 1151 (see, e.g., component 230 of FIG. 1C), a right PPG sensor 1152 (see, e.g., component 222 of FIG. 1C), a right temperature sensor 1153 (see, e.g., component 240 of FIG. 1C), a right occlusal pressure sensor 1154 (see, e.g., component 211 of FIG. 1C), and/or the like, all of which may be intercoupled by any suitable internal module bus(ses) 1158. In some embodiments, a single central IMU (e.g., IMU 230) may be sufficient and may be positioned centrally for compactness and simplified calibration. Additionally or alternatively, multiple IMUs 1141/1151 may be provided on opposite sides of the dental arch to improve spatial resolution, directional sensing, redundancy, and/or local motion characterization. Accordingly, FIG. 2 may show a representative central IMU embodiment, whereas FIG. 10 may show a modular multi-IMU embodiment. A left or right IMU may include both an accelerometer and a gyroscope within a single package. In other embodiments, separate side-specific accelerometers and gyroscopes may be used. Similarly, a single central temperature sensor (e.g., sensor 240) may be sufficient in some embodiments. Additionally or alternatively, left and right temperature sensors 1143/1153 may be used in other embodiments to improve local measurement resolution and/or redundancy. It may be appropriate to include left and right occlusal pressure sensors 1144/1154 in lingual sensor modules 1140/1150 where the module placement may be anatomically compatible with force transfer measurement and integrated sensor routing. The term “lingual sensor module” may refer to any suitable module placement (e.g., along the inner side of the arch) and does not preclude the module from including force-sensing elements associated with occlusal loading.

System 1100 may include any suitable flexible wiring harness (e.g., via sensor bus(ses) 1171, stimulation signal line(s) 1172, power distribution line(s) 1173, and/or the like) that may be configured to interconnect all modules (see, e.g., interconnect 114 of FIG. 1C).

Interconnect 114 of FIG. 1C and FIG. 2 may correspond functionally to the flexible wiring harness that may be represented by buses 1171, 1172, and 1173 of FIG. 10, although FIG. 10 may illustrate a more modular bus-based implementation.

Central LED 433 of FIG. 2 may be represented in FIG. 10 as part of electronics capsule 1110, as part of a dedicated front visual indicator submodule, or as a centrally located optical output element coupled to LED driver circuitry.

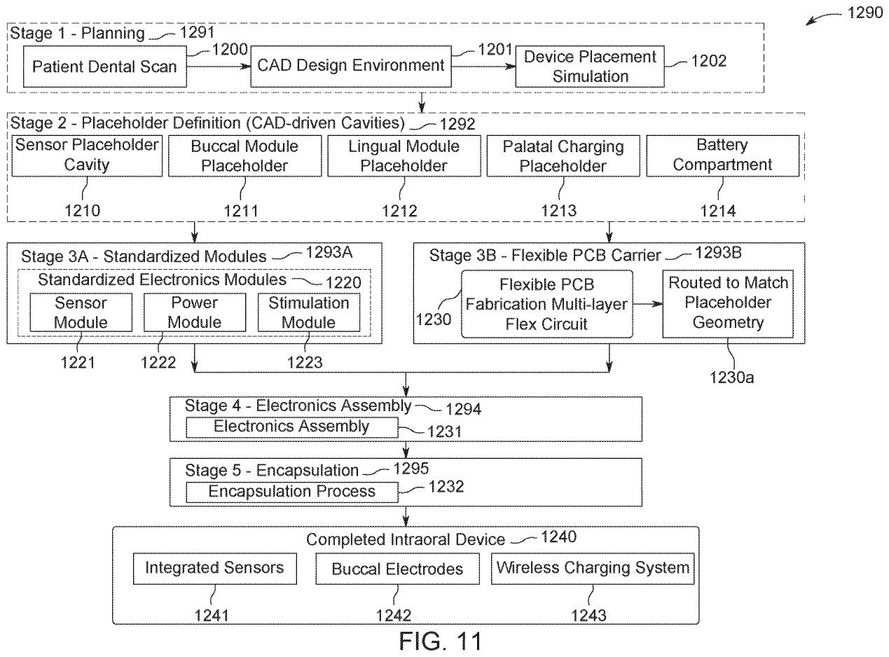

FIG. 11 illustrates an exemplary workflow process 1290 for manufacturing an intraoral device 1240. Intraoral device 1240 of FIG. 11 may correspond generally to apparatus 102 of FIGS. 1 and 1B, subsystem 100 of FIGS. 1C, 2, and 8, and/or an assembled implementation of modular system 1100 of FIG. 10.

As shown in FIG. 11, a first stage 1291 of process 1290 may be a planning stage and may include any suitable operations, including, but not limited to, operation 1200, operation 1201, operation 1202, and/or the like. At operation 1200 of first stage 1291, any suitable patient dental scan (e.g., any suitable 3D scan and/or analog impression) may be taken of any suitable user (e.g., a mouth or other suitable portion thereof), or where a standard size (e.g., small, medium, large) may be chosen if patient personalization is to be skipped. Next, at operation 1201 of first stage 1291, any suitable CAD design may be carried out in any suitable CAD design environment (e.g., using any suitable parametric device modeling or otherwise). Next, at operation 1202 of first stage 1291, any suitable device placement simulation may be carried out (e.g., fit verification, clearance check, and/or the like). Stage 1291 may provide various unique solutions, including, but not limited to, defining device architecture from a dental scan while preserving compatibility with standardized electronics modules, simulation of both anatomical fit and stimulation-field-relevant placement, planning of component positions based on both sensing objectives and neuromodulation target geometry, and/or the like.

As shown in FIG. 11, a second stage 1292 of process 1290 may be a placeholder definition stage (e.g., a CAD-driven cavities defining stage) and may include any suitable operations, including, but not limited to, operation 1210, operation 1211, operation 1212, operation 1213, operation 1214, and/or the like. At operation 1210 of second stage 1292, any suitable sensor module placeholder cavity may be defined for any suitable respective sensor or sensor module (e.g., any suitable recess 117 of FIG. 1B). At operation 1211 of second stage 1292, any suitable buccal module placeholder cavity may be defined for any suitable respective buccal electrode or actuator or indicator or buccal module (e.g., any suitable recess 117 of FIG. 1B). At operation 1212 of second stage 1292, any suitable lingual module placeholder cavity may be defined for any suitable respective lingual sensor or lingual sensor module (e.g., any suitable recess 117 of FIG. 1B). At operation 1213 of second stage 1292, any suitable palatal charging placeholder cavity may be defined for any suitable respective palatal charging component or palatal charging module (e.g., any suitable recess 117 of FIG. 1B). At operation 1214 of second stage 1292, any suitable battery compartment or placeholder cavity may be defined for any suitable respective battery component or battery module (e.g., any suitable recess 117 of FIG. 1B). The various types of placeholders, cavities, recesses, and/or the like that may be defined at second stage 1292 may be determined based on the planning achieved at first stage 1291 (e.g., from any suitable digital scan, analog impression, standardized multi-size shell, etc. (e.g., for providing any suitable universal inner cavities)). Stage 1292 may provide various unique solutions, including, but not limited to, CAD-driven definition of patient, shell-specific placeholder geometries that may be functionally decoupled from mass-produced electronics modules, enabling scalable manufacturing with reusable electronics and variable shell geometries, and/or the like. The specific placeholder types may be discretely itemized (e.g., as placeholders 1210-1214) because they may correspond to distinct functional categories having different spatial, mechanical, and electrical constraints, including sensing, actuation, charging, and energy storage. Sensor module placeholders (e.g., a placeholder 1210) may correspond to regions associated with sensors 210, 211, 221, 222, 230, 240, and/or the like. Buccal module placeholders (e.g., a placeholder 1211) may correspond to regions associated with electrodes 411-414, actuators 420/421, LEDs 431-433, and/or the like. Lingual module placeholders (e.g., placeholder 1212) may correspond to inner-arch sensor, module regions, and/or the like. Charging placeholders (e.g., a placeholder 1213)may correspond to charging coil 611, related subsystem 600 regions, and/or the like. Battery placeholders (e.g., a placeholder 1214) may correspond to battery 510/511 regions and/or the like.

As shown in FIG. 11, a first-third stage 1293A of process 1290 may be a standardized modules definition stage (e.g., a patient-independent, mass-produced, standardized modules creation stage) for creating any suitable standardized electronics modules fabrication at operation 1220. At operation 1220 of first-third stage 1293a, any suitable modules may be fabricated, including, but not limited to, any suitable sensor module(s) 1221, any suitable power module(s) 1222, any suitable stimulation module(s) 1223, and/or the like. Stage 1293A may be performed independently of a specific patient geometry, but may use the functional module definitions established conceptually in earlier stages to ensure compatibility with the placeholder architecture. Sensor module(s) 1221 may include force sensors, physiological sensors, inertial sensing modules, and/or the like. Power module(s) 1222 may include batteries, charging components, protection circuitry, and/or the like. Stimulation module(s) 1223 may include electrodes, driver circuitry, vibration actuators, visual indicator elements, and/or the like. Stage 1293A may provide various unique solutions, including, but not limited to, mass-produced patient-independent modules configured for integration into patient-specific or standardized shell geometries without redesign of the module electronics, and/or the like.

As shown in FIG. 11, a second-third stage 1293B of process 1290 may be a flexible PCB carrier definition stage for creating any suitable flexible PCB fabrication at operation 1230. At operation 1230 of second third stage 1293B, any suitable flexible PCB may be fabricated (e.g., any suitable multi-layer flex circuit(s)). Then, at operation 1230a of second-third stage 1293B, the fabricated flexible PCB may be routed to match any suitable placeholder geometry. Placeholder geometry may be defined in stage 1292. Stage 1293B may provide various unique solutions, including, but not limited to, routing a flexible PCB carrier according to CAD-defined anatomical placeholder geometry so that a common electronics architecture can be adapted to multiple shell geometries, and/or the like. The flexible PCB carrier may correspond to carrier structure 110, flexible interconnect 114, the contoured carrier architecture described in FIGS. 1C, 2, and 8, and/or the like. Stages 1293A and 1293B may be performed independently in fabrication, but may converge in assembly stage 1294.

As shown in FIG. 11, a fourth stage 1294 of process 1290 may be an electronics assembly definition stage for creating any suitable electronics assembly at operation 1231. At operation 1231 of fourth stage 1294, any suitable electronics assembly may be generated (e.g., any suitable module(s) (e.g., as defined at stage 1293A) may be placed into a flex PCB and/or carrier (e.g., one or more of modules 1221, 1222, 1223 may be placed in a placeholder cavity) per placeholder layout (e.g., as defined at stage 1293B)). Stage 1294 may include placement, alignment, electrical coupling, and mechanical integration of standardized modules into the flexible carrier or corresponding placeholder layout. Stage 1294 may provide various unique solutions, including, but not limited to, assembly of standardized reusable modules into a geometry-specific carrier arrangement defined independently from the module fabrication process, and/or the like. This electronics assembly may correspond to the integrated component arrangement represented functionally in FIG. 1C and spatially in FIGS. 2 and 8.

As shown in FIG. 11, a fifth stage 1295 of process 1290 may be an encapsulation definition stage for creating any suitable encapsulation at any suitable encapsulation process of an operation 1232. At operation 1232 of fifth stage 1295, any suitable portion or the entirety of the electronics assembly (e.g., as generated at fourth stage 1294) may be encapsulated (e.g., using any suitable biocompatible sealing (e.g., hermetic sealing)). Encapsulation stage 1295 may include overmolding, sealing, moisture protection, electrical insulation, selective exposure of functional interfaces (e.g., electrode contact surfaces or optical windows), and/or the like. Stage 1295 may provide various unique solutions, including, but not limited to, selective encapsulation of a modular electronics assembly such that biocompatible sealing may be achieved while preserving exposed contact interfaces and compatibility with reusable module concepts, and/or the like. This encapsulation may correspond to encapsulation layer 115, sealing layer 116, overmold 1020, and/or the like.

As shown in FIG. 11, after fifth stage 1295 of process 1290, a completed intraoral device 1240 may be provided (e.g., a patient-specific or standardized one of multi-size shells and standardized electronics that may be ready for end use within a patient user). Such a completed intraoral device 1240 may include any suitable integrated sensor(s) 1241, any suitable buccal electrode(s) 1242, any suitable wireless charging system 1243, and/or the like. These components may be listed as representative examples rather than an exhaustive set, highlighting key sensing, stimulation, and charging functions of completed device 1240. Completed device 1240 may correspond generally to apparatus 102, subsystem 100, and/or an assembled implementation of system 1100.

Process 1290 of FIG. 11 may be configured to support patient-specific or standardized shells. A shell geometry (e.g., as may be defined at stages 1291 and 1292) may be decoupled from standardized electronics (e.g., as may be defined at stage 1293A), as modules generated at operation 1220 may be mass-produced independently. A final assembly of stage 1294 for encapsulation at stage 1295 may combine both a track via stage 3A and a track via stage 3B for providing a completed device 1240. In some embodiments, standardized modules may be manufactured independently from patient-specific carrier structures and may be replaceable across multiple devices. Such an approach may enable servicing, upgrading, and reuse of functional components without requiring replacement of the entire intraoral device (e.g., modular production, shells and electronics).

It is understood that the operations shown in process 1290 of FIG. 11 are only illustrative and that existing operations may be modified or omitted, additional operations may be added, and the order of certain operations may be altered. Carrier structure 110, shell 102s, and/or the like may be represented in the shell or final device geometry surrounding device 1240. Encapsulation layer 115, sealing layer 116, overmold 1020, and/or the like may be represented by the encapsulated body of device 1240 after stage 1295. Circuitry 102c may correspond to the assembled electronics resulting from stages 1293A, 1293B, and 1294. FIG. 11's illustration of device 1240 may be supplemented with a 3D exploded or assembled illustration or the like, such as analogous to FIG. 1B, FIG. 2, FIG. 8, and/or the like.

Therefore, the disclosure may provide an intraoral subsystem (e.g., neuromodulation platform) 100 that may include various functional subsystems integrated into a compact, tooth-supported structure that may be configured for placement within an oral cavity of a user (e.g., FIG. 1C may schematically illustrate an exemplary embodiment of such an overall system architecture of subsystem 100).

While the intraoral subsystem may preferably be implemented as an intraoral device, a sensing subsystem thereof may alternatively or additionally cooperate with external devices that may be positioned outside the oral cavity. Such external devices may include wearable units configured to provide actuation or additional sensing functionality. Examples of such external wearable units may include, but are not limited to, smart watches, ear-worn devices, headbands, skin patches, chest straps, smart glasses, helmet-integrated units, and/or the like. Such wearable units may provide additional sensing, including, but not limited to, heart rate, acceleration, head impact, skin conductance, respiration, temperature, and/or contextual activity sensing, and/or may provide additional actuation, such as haptic alerts, audible alerts, and/or remote visual signaling. Such external devices may further provide location information, including GPS-based positioning, associated with the intraoral device (e.g., when the external device and the intraoral device are maintained withing a threshold distance of each other (e.g., to support BLE, etc.)). An external device (e.g., device 1000) may be configured to determine proximity of the intraoral device based on received signal strength and may provide location data, including GPS-based location information, associated with the intraoral device. In some embodiments, the intraoral device may include a locating subsystem configured to enable detection of the device when not worn. The locating subsystem may utilize a wireless communication interface (e.g., BLE) to transmit periodic beacon signals. The locating subsystem may operate in a reduced duty cycle mode to minimize power consumption. The intraoral device may be configured to determine an operational state including a wear state, a docked state, and a non-wear state, wherein the locating subsystem may be activated in the non-wear state.

The disclosure further describes a platform architecture for intraoral sensing, monitoring, and intervention technologies, wherein any suitable number of sensing elements, processing elements, and intervention elements may be integrated within or associated with an oral interface structure. The platform architecture may enable detection of oral events, physiological conditions, behavioral patterns, and/or impact-related events and provide corresponding device responses, including, but not limited to, feedback, neuromodulation, monitoring, and/or signaling functions.

In some embodiments, such an intraoral subsystem 100 may include carrier structure 110 that may be adapted to be positioned on teeth of an upper jaw, a lower jaw, or both. Carrier structure 110 may support a plurality of electronic and electromechanical components that may be arranged according to a predefined spatial configuration.

The system architecture may be modular and may include any suitable functional domains, including, but not limited to, sensing domain 200 that may be configured to acquire mechanical, kinematic, and physiological parameters, P&C domain 300 that may be configured to process acquired signals and exchange data (e.g., wirelessly), actuation domain 400 that may be configured to provide mechanical, electrical, and/or visual feedback, power management domain 500 that may be configured to supply energy to other respective domains or component(s) thereof, safety domain 800 that may be configured to enforce operational limits (e.g., independently of software execution), and/or the like. In some embodiments, one or more functional domains or modules may be configured as removable and replaceable components. Such modules may include sensing modules (see, e.g., domain 200), processing and communication modules (see, e.g., domain 300), actuation modules (see, e.g., domain 400), power modules (see, e.g., domain 500), and/or the like. At least some of these modules may be configured as reusable, patient-independent components that may be installed into different carrier structures (e.g., for providing a modular system).

In some embodiments, P&C domain 300 may include microcontroller unit 310 that may include wireless communication interface 320, such as BLE interface 321, where microcontroller unit 310 may be configured to receive signals from sensing domain 200 and to provide control signals to actuation domain 400. Microcontroller unit 310 might not be relied upon to enforce safety-critical limits of electrical stimulation subsystem 410 of domain 400. External software, wireless commands, and/or user interaction might not be able to increase stimulation parameters beyond hardware-enforced limits.

A key aspect of the disclosure is architectural separation between processing domain 300 and safety domain 800 (see, e.g., FIG. 3). Electrical stimulation (e.g., of stimulation driver architecture 700) may be generated by stimulation subsystem 410, which may be galvanically isolated from processing domain 300 and from external communication interfaces.

All safety-critical limits associated with stimulation subsystem 410 may be enforced by dedicated hardware components rather than by software. This may include at least one hard current limit, at least one hard DC-fault limit, and at least one latched shut-down path that cannot be overridden by firmware execution.

Safety domain 800 may include hardware current limiting circuit(s) 810, hardware voltage monitoring circuit(s) 820, isolation barrier(s) 720, and latch circuits 840 configured to disable stimulation in response to detected fault conditions, including over-current, DC offset, watchdog timeout, and/or abnormal stimulation capacitor polarization. Safety domain 800 may be described as including or cooperating with barrier 720 in a functional sense, in that barrier 720 may participate in the safety architecture by preserving isolation between control and patient-side domains. However, barrier 720 may alternatively or additionally be described as a distinct isolation component associated with, rather than wholly contained within, safety domain 800.

Electrical stimulation as used herein may be configured to influence sensory afferent pathways, motor pathways, and/or combinations thereof, such as without requiring an explicit muscle contraction. Such stimulation may be configured as neuromodulation, sensory interruption, conditioning feedback, and/or other controlled excitation of tissue. An explicit muscle contraction may refer to a visible or force-generating contraction (e.g., macroscopic contraction) of one or more muscles, such as a contraction sufficient to produce measurable jaw movement, tissue displacement, or bite-force generation (e.g., as opposed to sub-threshold modulation of neural activity). Neuromodulation may refer to controlled stimulation that may be configured to alter neural or neuromuscular signaling without necessarily producing a direct or sustained muscle contraction (e.g., controlled electrical stimulation configured to alter neuromuscular activity or sensory signaling without inducing a direct or sustained muscle contraction). Sensory interruption may refer to temporary disruption, attenuation, or modulation of sensory afferent signaling associated with an oral event or bruxism-related activity. Conditioning feedback may refer to repeated stimulus delivery configured to influence user behavior or neuromuscular response patterns over time. Controlled excitation may refer to targeted activation of tissue or neural pathways (e.g., using stimulation parameters that may be maintained within predefined safe operating limits).

Physiological parameters may include direct measurements or proxy/trend-based indicators that may correlate with oxygenation, perfusion, or respiratory-related variation. In particular, oxygen-related parameters may be evaluated as trends and correlated indicators rather than as absolute diagnostic SpO₂ values. Escalation stages or intervention stages described herein may represent predefined device responses and might not constitute clinical decision-making. Intraoral subsystem 100 may be configured to provide condition-based feedback and alerts without performing medical diagnosis.

Selective stimulation geometry may be employed in which electrode placement and field shaping may be configured to preferentially affect target regions associated with mastication musculature while minimizing spread toward undesired nerve pathways.

Sensing domain 200 may include one or more pressure sensors (e.g., sensor 210 and/or sensor 211) that may be configured to detect occlusal forces generated between teeth (e.g., to detect occlusal loading). Pressures sensors 210/211 may be configured to detect occlusal force transmitted between opposing upper and lower teeth, particularly in premolar and molar regions. Such forces may be oriented substantially along the Z-axis during clenching, bruxism, or impact-related jaw loading. Such pressure sensors may be positioned at molar and/or premolar regions, including bilateral positioning for right/left balance assessment.

Additionally or alternatively, sensing domain 200 may include one or more physiological sensing modules or physiological sensors 220 that may be configured to detect any suitable parameters, such as photoplethysmographic signals, pulsatile blood flow, pulse rate, pulse rate variability, oxygen-related proxy trends, and/or combinations thereof, such as through contact with oral mucosa. In some embodiments, such physiological sensors may include left PPG sensor 221 and right PPG sensor 222. Sensor 221/222 may be positioned along palatal or lingual-facing regions adjacent to vascularized mucosal tissue (e.g., for contacting oral mucosa), thereby improving optical coupling and physiological signal quality while minimizing interference with occlusal loading regions.

Additionally or alternatively, sensing domain 200 may include at least one inertial sensing module 230, acceleration sensor 231, and/or gyroscope 232 that may be configured to detect linear and/or angular acceleration of an intraoral device. An acceleration sensor may include a multi-axis accelerometer 231 optionally combined with a gyroscopic sensor 232.

Acceleration sensor 231 may be configured to detect rapid acceleration events indicative of impacts, sudden movements, and/or collisions affecting the user. Acceleration data may be evaluated independently or in combination with pressure sensor data from one or more pressure sensors (e.g., sensors 210, 211, etc.).

In some embodiments, subsystem 100 may be configured to interpret simultaneous detection of acceleration exceeding a predefined threshold and occlusal force exceeding a predefined threshold as a high-risk event.

In some embodiments, event detection may be based on a single sensing modality. In other embodiments, event detection may be based on sensor fusion using multiple sensing modalities. In some embodiments, event detection may include thresholding of occlusal force and inertial motion, temporal correlation of threshold exceedance, consistency evaluation across multiple detection cycles, suppression of event classification when conflicting or non-correlated signals are detected, severity scoring based on force magnitude, repetition rate, and motion intensity, adaptive adjustment of thresholds or timing windows (e.g., based on historical user-specific data), and/or the like. Such processing may distinguish normal chewing activity from bruxism, clenching, or impact-related high-risk events.

Actuation domain 400 may include one or more vibration actuators (e.g., actuator 420, actuator 421, etc.) that may be configured to generate any suitable mechanical stimuli that may be perceptible to user that may be intraorally wearing subsystem 100.

Actuation domain 400 may additionally or alternatively include electrical stimulation subsystem 410 that may be configured to deliver biphasic stimulation pulses through one or more electrodes (e.g., buccal electrode(s) 411, 412, etc.) that may be configured to contact oral tissue when subsystem 100 is worn intraorally. Electrodes 411/412 may be positioned along buccal-facing portions of carrier structure 110 such that their contact surfaces may be exposed through or beyond the encapsulation in defined tissue-contact windows near boundary 110b, thereby enabling direct or near-direct contact with buccal tissue while the remaining structure remains sealed and mechanically protected.

Actuation and/or intervention subsystem 400 may provide electrical stimulation, mechanical stimulation, vibrotactile stimulation, acoustic stimulation, optical stimulation, and/or combinations thereof. Different stimulation modalities may be enabled by a combination of different actuator or output element types, different signal types or waveforms, different temporal patterns, different spatial positioning along carrier structure 110, different combinations of simultaneously or sequentially activated outputs, and/or the like. Therefore, coordinated multi-modal intervention selected on the basis of multi-sensor event detection within a safety-constrained intraoral platform may be provided. In some embodiments, electrical stimulation may be omitted and the intervention subsystem may operate using mechanical or vibrotactile feedback alone. In further embodiments, the intervention subsystem may combine multiple feedback modalities.

In some embodiments, actuation domain 400 may include at least one visual signal indicator 430, such as one or more LEDs 431/432, which may be configured to provide any suitable visible signaling that may correspond to detected events or conditions for alerting any suitable viewer. Visual signal indicator 430 may provide different colors, intensities, and/or patterns to indicate different severity levels of a detected event and/or different types of detected events and/or different types of status (e.g., a normal status, caution status, or alert status based on the defined oral event and/or a computed severity level). In some embodiments, a red visual signal state may indicate that a predefined force threshold, acceleration threshold, or combined risk threshold has been exceeded. Visual signal indicator 430 may be positioned to be visible externally while the intraoral device is worn, enabling recognition by third parties without access to electronic data. Haptic and/or audible indicators may be used in addition to or as an alternative to visual indicators. For example, local warning may be provided by vibration actuators, miniature acoustic transducers, extraoral wearable haptic outputs, external device notifications, and/or the like.

Intraoral neuromodulation system 100 may include an energy storage element 510 that may be configured as a single-cell lithium-based rechargeable battery 511. This may supply electrical energy to at least processing, sensing, communication, and actuation components of the system. Such a battery may be selected and integrated such that its nominal operating voltage may remain below a predefined low-voltage threshold, thereby reducing electrical hazard potential in the intraoral environment. Battery protection circuitry 520 may include over-voltage protection 522, under-voltage protection 523, over-current protection 521, short-circuit protection 541, and/or the like, which may be enforced by hardware mechanisms independent of firmware execution. Integrating hardware-enforced battery and stimulation safety mechanisms into a multi-domain intraoral neuromodulation system may provide unique solutions in which safety limits may remain independent of firmware execution (e.g., hardware independent battery protection).

Intraoral neuromodulation system 100 may include temperature monitoring circuitry (e.g., temperature sensor 240 and/or thermal protection circuitry 850), which may be associated with the battery. Upon detection of an over-temperature condition, at least one of charging, discharging, and/or electrical stimulation may be disabled by hardware. The battery may be mechanically supported and encapsulated within carrier structure 110 (e.g., using any suitable elastomeric and/or polymeric materials, such as for providing at least moisture resistance, electrical insulation, and/or mechanical buffering 115 and/or 116).

In some embodiments, battery charging may be disabled or limited while the device is positioned intraorally. Charging may be restricted to non-wear conditions and/or limited such that excessive temperature rise may be prevented. Charging may be disabled or limited automatically using wear-state detection, impedance sensing, temperature monitoring, docking-state detection, motion analysis, and/or any suitable hardware-detected conditions that may indicate intraoral use or unsafe charging circumstances.

Intraoral neuromodulation system 100 may include a multi-domain electrical architecture that may be configured to separate low-voltage control electronics from patient-contacting stimulation circuitry (e.g., architecture 390 separating domain 391 from domain 399).

In some embodiments, at least a portion of the sensing subsystem, processing subsystem, safety subsystem, and/or stimulation subsystem may be implemented within an integrated circuit device. Such integrated circuit devices may include application-specific integrated circuits (“ASICs”), system-on-chip (“SoC”)architectures, and/or mixed-signal integrated circuits that may be configured to implement at least part of the described platform architecture.

A first electrical domain (e.g., domain 391) may be configured to supply processing, sensing, and communication components at a low voltage level. A second electrical domain (e.g., domain 399) may be configured to supply stimulation components at a higher voltage level suitable for biphasic stimulation. The second electrical domain may be galvanically isolated from the first electrical domain. Isolation barrier 720 may be implemented using transformer-based isolated converters 710, digital isolators, hardware-assisted digital circuits, optocouplers, and/or combinations thereof. The stimulation domain (e.g., domain 399) may include an isolated ground reference (e.g., ISO_GND) that may be distinct from a control ground reference (e.g., GND). Such an isolated ground reference may be shown in FIGS. 3 and 7 as a reference node within the isolated stimulation domain distinct from the control-domain ground. Such isolation may be configured to ensure that stimulation-domain faults cannot propagate into processing domain 300. Fault propagation may be prevented by galvanic isolation, isolated power transfer, isolated signaling, and/or absence of direct conductive return paths between the stimulation domain and the low-voltage processing domain.

A stimulation domain (e.g., domain 399) may include hardware current monitoring 810, hardware DC-fault monitoring 820 across a DC-blocking capacitor 750, and a latch-based shut-down path 840 that may force stimulation to an off state upon detection of a fault condition.

Safety enforcement may be implemented using hardware circuits, mixed hardware-software architectures, and/or combinations thereof, provided that stimulation output parameters cannot exceed predefined limits. Such limits may be predefined by hardware component selection, resistor values, comparator thresholds, capacitor sizing, watchdog timing, fixed analog or mixed-signal safety thresholds, and/or the like (e.g., maximum stimulation current, maximum pulse width, maximum repetition rate, maximum burst duration, maximum duty cycle, maximum cumulative delivered charge, maximum allowable DC offset, etc.). Hardware safety enforcement subsystem or safety domain 800 (e.g., as may be operating within a stimulation subsystem 410) may be configured to enforce stimulation output constraints (e.g., independently of firmware and/or independent of a sensing and control subsystem (e.g., domain 200 and/or domain 300)). Such stimulation output constraints may include limits for current, voltage, pulse width, repetition rate, burst duration, and duty cycle enforced by hardware circuits. An isolated supply may be provided by transformer-based isolation or capacitive isolation. Electrode polarization may correspond to sustained DC bias at the electrode-tissue interface. A hardware-validated reset sequence may involve a physical or hardware-detected action that may not be achievable solely by software.

Hardware watchdog 830 may be employed such that absence of a periodic signal may result in stimulation shutdown. Watchdog supervision may be transferred across isolation barrier 720 and may participate in the same latched shut-down mechanism 840. This may be shown in FIGS. 3, 7, and 9 as a supervisory signal path crossing barrier 720, whereby absence or invalid timing of a permitted request may cause hardware shutdown within the isolated domain.

For example, FIG. 3 and FIG. 7 may illustrate exemplary implementations of electrical domain separation and hardware safety enforcement. FIG. 9 may also illustrate the separation between software-controlled logic and hardware-enforced safety. However, FIG. 9 may emphasize compliance and control authority separation, whereas FIGS. 3 and 7 may more directly emphasize electrical isolation and stimulation-path safety enforcement.

System 100 may be configured for placement in an upper jaw, lower jaw, or both.

The intraoral device may be implemented in various form factors, including, but not limited to, dental splints, mouthguards, retainers, orthodontic appliances, bite plates, partial dental structures, and/or the like. In further embodiments, the electronics architecture may be integrated into removable modules, partially embedded structures, or fully encapsulated electronic assemblies. In further embodiments, sensing components may remain intraoral while intervention components may be located intraorally or extraorally. Examples of extraoral intervention components may include, but are not limited to, external haptic wearables, audible alert devices, visual indicators on an external device, ear-worn feedback units, extraoral stimulation units communicatively coupled to the intraoral subsystem, and/or the like. Such extraoral outputs may interface with the user through skin contact, auditory pathways, visual displays, wearable accessories, and/or the like.

Electronic components, conductive paths, interconnections, and mechanical interfaces may be defined as spatial placeholders within a CAD environment, decoupling electronics from patient-specific geometry. The electronic components may be arranged as modular assemblies, partially integrated assemblies, or fully integrated monolithic assemblies within the carrier structure. The same electronic assembly may be integrated into patient-specific shells (e.g., shells 111, 112, etc.) or standardized shells of predetermined sizes and shapes without redesign of the electronic design. In some embodiments, carrier structure 110 (e.g., a carrier plate) may be a flexible printed circuit board contoured to follow anatomical structures. Components may be mounted thereto using any suitable surface-mount technology and subsequently encapsulated using any suitable biocompatible materials (e.g., materials 115, 116, etc.) while electrode contact surfaces (e.g., electrode(s) 411, 412, etc.) may remain exposed. Electrode contact surfaces may remain selectively exposed through defined openings, windows, uncovered tissue-contact regions, and/or the like in one or both of layers 115 and 116, such that only the intended contact surface may be exposed for interaction with buccal tissue while surrounding conductive and electronic regions may remain encapsulated. Patient protection may be ensured by biocompatible edge sealing, controlled contact surface geometry, galvanic isolation, hardware-enforced stimulation limits, smooth surface finishing, limitation of exposed conductive area to intended electrode interfaces, and/or the like. Predefined cavities or recesses 117 in a shell 111 and/or carrier structure 110 may correspond to placeholders (e.g., CAD placeholders) and may provide repeatable insertion positions for scalable manufacturing. FIG. 2 illustrates exemplary placement within an upper jaw including buccal electrode positioning and component zones including battery placement.

Intraoral neuromodulation system 100 may be referred to herein as a multifunctional platform (e.g., a platform of a wider BMS platform) that may be capable of sensing, monitoring, and providing feedback within a user's oral cavity. In some embodiments, the system may be configured to monitor occlusal force parameters, including peak force, average force, accumulated force exposure, right/left balance metrics, and/or the like using any suitable sensors (e.g., force sensing elements 210, 211, etc.). The system may be configured to monitor any one or multiple suitable force-related characteristics, including, but not limited to, peak occlusal force (e.g., along the Z-axis between opposing upper and lower teeth), sustained clenching force over time, repetitive force bursts (e.g., associated with grinding), right/left asymmetry between bilateral posterior regions, force accumulation over repeated cycles, transient high-load events (e.g., associated with impact or collision), and/or the like. Placement of posterior occlusal force sensors in premolar regions and/or molar regions may enable detection of high-force biting and clenching. Bilateral placement may enable asymmetry analysis. Inertial sensing may complement force sensing by distinguishing static clenching from dynamic impact-related loading.

Event detection and classification may be implemented using any suitable deterministic algorithms, threshold-based detection, statistical evaluation, machine learning models, artificial intelligence models, and/or combinations thereof (e.g., any suitable application(s) 19a, model(s) 19m, and/or the like of any suitable controller 310 or otherwise of the system). In some embodiments, the system may be configured to implement any suitable adaptive learning (e.g., based on historical sensor data collected from the individual user). The processing subsystem may dynamically adjust detection thresholds, temporal correlation windows, classification parameters (e.g., based on observed patterns of occlusal force, motion characteristics, event frequency, and/or the like), and/or the like. Such adaptation may be implemented using any suitable statistical analysis, rule-based adjustments, machine learning models trained on user-specific data, and/or the like. The system may continuously refine its detection model to reduce false positives, improve classification accuracy, personalize intervention timing and intensity, and/or the like. Adaptive learning may further include weighting of sensor modalities, adjustment of feature importance, recalibration of severity metrics based on cumulative exposure or behavioral trends, and/or the like. The adaptive learning process may operate within predefined safety constraints and parameter boundaries to ensure that adjusted thresholds remain within safe and clinically acceptable ranges. Adaptation may be validated against predefined criteria to prevent unsafe or unstable system behavior. The adaptation may directly affect physical device operation by modifying stimulation triggering conditions and intervention timing. An occlusal force threshold may correspond to a level exceeding normal mastication forces and may be defined as a patient-specific or adaptive threshold that may be determined during calibration. In some embodiments, the system may be configured to compute an accumulated stimulation dose metric representing cumulative neuromodulation exposure over a defined time period. The dose metric may include any suitable parameters, including, but not limited to, total delivered charge, stimulation duration, intensity, frequency of stimulation events, and/or the like. In some embodiments, the accumulated dose metric may be used to prevent overstimulation by limiting total stimulation exposure within a predefined time window. An escalation controller may use this dose metric to limit excessive stimulation, enforce cooldown intervals, and/or adjust intervention strategies to maintain safety and optimize therapeutic outcomes.

Based on detected parameters, the system may provide staged escalation feedback. In a first stage, vibration feedback may be provided via vibration actuators (e.g., actuators 420, 421, etc.). In a second stage, electrical stimulation may be provided via stimulation subsystem 410.

In a further stage, combined vibration and electrical stimulation may be provided. Cool-down and lockout intervals may be applied.

A staged escalation protocol may represent predefined device responses and may not constitute medical diagnosis or clinical decision-making.

In some embodiments, physiological monitoring may include oxygen-related proxy trends and/or photoplethysmographic trend features that may be provided by any suitable physiological sensing elements (e.g., element(s) 220, 221, 222, etc.). If a trend parameter remains outside acceptable limits despite escalation, an external alert may be transmitted automatically to an external device 1000. External device 1000 may be configured to automatically initiate contact with a caregiver or emergency service subject to configuration and regulatory requirements.

FIG. 5 illustrates an exemplary functional flow 599 for staged escalation based on detected mechanical and/or physiological parameters.

In some embodiments, the system may be configured for impact and contact-sport monitoring. Acceleration events indicative of impacts may be detected and evaluated in combination with occlusal force. If a high-risk condition is detected, a visible signal (e.g., signal with one or more indicators 430, 431, 432, 433, etc.), such as a red LED, may be activated such that a coach, trainer, referee, or supervisor may recognize the condition.

Detected thresholds and accumulated exposure metrics may be transmitted to an external application 1010 that may be configured to display or otherwise present threshold status, cumulative exposure, and/or a recommendation that a player be substituted, removed from participation, or subjected to further evaluation.

Data generated by the system may be transmitted to external computing devices, mobile applications, cloud-based systems, and/or remote monitoring platforms. External systems may analyze device data to derive behavioral trends, health-related indicators, or usage patterns.

FIG. 6 illustrates an embodiment for impact monitoring including acceleration sensing 231, force sensing 210, 211, visual signaling 430, and external application interaction 1010.

In non-limiting examples, stimulation pulses that may be generated by stimulation subsystem 410 may be biphasic, such as with phase durations in a range of 20 μs to 2 ms, pulse repetition frequencies in a range of 1 Hz to 200 Hz, and burst durations in a range of 50 ms to 5 s. Hardware-enforced peak stimulation current limits may be selected in a conservative intraoral range, for example 0.5 mA to 10 mA, with an additional independent series resistance 740 and independent comparator threshold 810 that may trigger latch shutdown 840.

DC-blocking capacitor 750 may be selected in a range of 0.47 μF to 10 μF, and DC-fault detection 820 may monitor capacitor polarization and/or mean electrode potential such that any sustained offset beyond a predefined threshold may be configured to trigger hardware shutdown.

Watchdog supervision interval 830 may be configured, for example, to use a periodic toggle within 50 ms to 2 s, where absence of the periodic signal may trigger latch shutdown 840.

Acceleration thresholds for impact monitoring may be configured based on sport type and population. In non-limiting examples, a threshold may be set based on peak g-level, integrated impulse, and/or repetition rate over time. Force thresholds may be set based on peak occlusal force and accumulated exposure.

Neuromodulation, as used herein, may refer to controlled electrical stimulation that may be configured to alter neuromuscular activity or sensory signaling without inducing a direct or sustained muscle contraction. An explicit muscle contraction may refer to a visible or force-generating contraction of a muscle group, as opposed to sub-threshold modulation of neural activity. As used herein, neuromodulation without inducing a direct muscle contraction may refer to stimulation levels and patterns that may influence neuromuscular signaling pathways without causing visible or measurable macroscopic muscle contraction or jaw displacement.

Such stimulation may operate below motor activation thresholds and may primarily target sensory afferent pathways or modulate reflex arcs. Detection based solely on occlusal force or solely on inertial motion may result in false positive or false negative event detection. The combined evaluation of occlusal force and inertial motion (e.g., sensor fusion) within a defined temporal window may improve specificity and robustness of event classification. Intraoral electrical stimulation may present elevated safety requirements (e.g., due to proximity to sensitive tissue). Hardware-enforced safety limits independent of firmware execution may reduce risk associated with software malfunction or communication errors. In case of any detected fault condition or invalid sensor state, the system may be configured to default automatically to a fail-safe mode in which stimulation is disabled. Event detection and intervention may be performed in real-time or near-real-time to ensure immediate system response to detected oral events. The combination of multi-modal sensing and adaptive thresholding may reduce false positive event detection compared to single-sensor systems. An occlusal force threshold may be defined as a patient-specific threshold exceeding normal mastication forces and may be calibrated during an initialization phase. An accumulated dose metric may include a time-integrated measure of occlusal force exposure, event frequency, and/or stimulation delivery. Electrode placement and geometry may be configured to preferentially target masticatory musculature while minimizing stimulation of undesired neural pathways. Electrode placement may be configured along the dental arch in proximity to anatomical regions associated with masticatory muscle activation, including regions adjacent to molars and premolars where force transmission is highest (e.g., in proximity to masticatory muscle insertion regions). Electrode geometry, spacing, and orientation may be selected to direct current flow toward target neuromuscular pathways while minimizing stimulation of undesired regions. Selectivity may be achieved by controlling current density distribution, electrode surface area, and inter-electrode distance (e.g., stimulation selectivity may be achieved through electrode geometry, spacing, and controlled current distribution). In some embodiments, multiple electrode configurations may be predefined and selectable based on detected event types to optimize neuromodulation effectiveness. In some embodiments, electrode configurations may be predefined and selectable based on detected event classes. A stimulation domain may operate on an electrically isolated supply and isolated reference potential separated by a galvanic isolation barrier.

In some embodiments, systems and methods of the disclosure may be directed not to conventional bruxism detection or biofeedback device, but rather to a multi-domain intraoral neuromodulation platform that may be characterized by multi-parameter sensor fusion, adaptive event classification, hardware-enforced safety architecture, and/or closed-loop intervention control. For example, rather than providing bruxism management or intraoral sensing that may rely on single-modality sensing (e.g., force-only or EMG-only), static thresholds, software-controlled feedback mechanisms, or simple alerting or vibration feedback, some systems and methods of the disclosure may provide combined evaluation of occlusal force and inertial motion within a predefined temporal window, dynamic classification of events based on multi-parameter sensor fusion, and suppression of false positives through signal consistency evaluation and conflict detection. This combination constitutes a structurally and functionally distinct system architecture than anything previously disclosed.

In some embodiments, systems and methods of the disclosure may utilize a multi-parameter sensor fusion model (e.g., any suitable model 19m) that may involve simultaneous or correlated exceedance of force and motion thresholds, temporal coincidence (e.g., <300 ms), and pattern consistency across detection cycles. This may not be a simple aggregation of known sensors, but rather a coordinated detection framework that may reduce false positive detections, distinguish physiological events from noise or normal activity, and enable classification of complex oral events. Importantly, such a system may also include conflict detection logic, wherein non-correlated signals may lead to suppression of event detection. Such bidirectional validation (e.g., confirmation and rejection) represents a non-obvious improvement in signal interpretation and reliability.

In some embodiments, systems and methods of the disclosure may not rely on fixed thresholds, but may include adaptive adjustment of thresholds, timing windows, and severity metrics, based on user-specific historical data, executed in real-time or near-real-time, and constrained by predefined safety limits and validated parameter ranges (e.g., adaptive learning with technical constraints). This may ensure that the system maintains safe operational boundaries, the adaptation produces a direct technical effect on device behavior, and the system improves detection accuracy over time. This is not an abstract data-processing feature, but a controlled, safety-bounded adaptation mechanism affecting physical system operation, which is absent in previously known intraoral devices.

In some embodiments, systems and methods of the disclosure may provide a hardware safety enforcement subsystem, which may include a primary current limiting circuit (e.g., an instance of hardware current monitor 810) and a secondary current limiting circuit (e.g., another instance of hardware current monitor 810), DC-offset detection (e.g., electrode polarization monitoring), watchdog supervision, and a fault latch enforcing a non-overridable shutdown state. Critically, safety limits may be independent of firmware, stimulation may default to an off-state, and reactivation may require hardware-validated conditions.

While some solutions may rely on software-controlled safety or lack multi-layer hardware enforcement, certain architecture of the disclosure may represent a fundamental structural difference, particularly relevant for intraoral neuromodulation where safety is critical. In some embodiments, each one of the primary current limiting circuit and the secondary current limiting circuit may be configured to trigger a simulation disabled state upon detection of overcurrent.

In some embodiments, systems and methods of the disclosure may provide a closed-loop intervention system that may include staged escalation (e.g., vibration into neuromodulation), severity-based decision-making, and an accumulated dose metric that may incorporate delivered charge, stimulation duration, frequency, and intensity. This may allow the system to dynamically regulate intervention, prevent overstimulation, and enforce cooldown and lockout intervals. Such dose-aware neuromodulation control represents a clinically relevant technical advancement for bruxism or intraoral devices (e.g., closed-loop escalation with dose-based control).

In some embodiments, systems and methods of the disclosure may provide functional decoupling of sensing, processing, safety, and stimulation domains, electrically and mechanically decoupled standardized electronics module, and independent hardware safety enforcement within an isolated stimulation domain (e.g., as opposed to modular intraoral components, or sensor-equipped mouthguards). Additionally, in some embodiments, systems and methods of the disclosure may define electrode placement and geometry for targeted neuromodulation, and operational constraints, such as wear-state validation and charging inhibition. These features go beyond structural modularity and define a coordinated system architecture with controlled interaction between domains.

In some embodiments, systems and methods of the disclosure are not limited to a single application (e.g., bruxism), but define a platform architecture capable of detecting multiple oral event types, supporting multiple intervention modalities, and operating across monitoring, behavioral conditioning, and neuromodulation domains. This may be enabled by the combination of sensor fusion, adaptive classification, and hardware safety enforcement. Such a generalized intraoral sensing and intervention platform is an advancement over application-specific systems.

Therefore, systems and methods of the disclosure may provide a distinct combination of technical features, including, but not limited to, multi-parameter sensor fusion with temporal correlation and conflict suppression, adaptive learning constrained by safety and applied in real-time, a hardware-enforced safety architecture independent of firmware, and a dose-aware, closed-loop neuromodulation control system. These elements collectively define a non-obvious, technically coherent, and safety-critical system architecture.

An intraoral neuromodulation system (100), comprising:
(a) a tooth-supported or dental-arch supported intraoral body (110, 111, 112) configured to be worn within an oral cavity of an individual user;
(b) a sensing subsystem (200) integrated within the intraoral body and comprising:
   at least one occlusal force sensing element (210, 211) configured to detect occlusal loading; and
   at least one inertial sensing element (230, 231, 232) configured to detect motion parameters comprising at least one of acceleration or angular velocity;
(c) a processing subsystem (300, 310, 330) configured to:
   receive sensor signals from the sensing subsystem; and
   detect an occurrence of a defined oral event based on a combined evaluation of occlusal loading and motion parameters of the received sensor signals within a predefined temporal window, wherein the defined oral event comprises a dynamically classified oral event based on multi-parameter sensor fusion;
(d) an intervention subsystem (400) configured to execute a device intervention in response to the detected occurrence of the defined oral event, the intervention subsystem comprising:
   a vibration output (420, 421); and
   a neuromodulation stimulation subsystem output (410) configured to alter neuromuscular activity without inducing a direct muscle contraction;
(e) an escalation controller (900, 950) configured to select between a plurality of intervention stages for the device intervention based on a severity metric derived from at least occlusal force magnitude and motion intensity, wherein the plurality of intervention stages comprises at least a first stage (950b) using the vibration output and a second stage (950c) using the neuromodulation stimulation subsystem output; and
(f) a safety subsystem (800) configured to:
   enforce stimulation output constraints using hardware-defined safety limits that are independent of the processing subsystem and not overrideable by firmware; and
   maintain a default-off stimulation state and require validation of multiple independent conditions prior to enabling stimulation, wherein:
      the hardware-defined safety limits comprise at least a hard current limit (810) and a fault-triggered latched stimulation disable state (840), and
      the defined oral event is determined using a multi-parameter sensor fusion model.

The system of paragraph [0191], wherein, when the intraoral body (110, 111, 112) is worn within the oral cavity, the sensing subsystem is positioned intraorally and the intervention subsystem is positioned intraorally or extraorally.

The system of paragraph [0191], wherein the defined oral event requires:
exceedance of an occlusal force threshold;
exceedance of an acceleration threshold, and
a temporal coincidence within less than 300 milliseconds, such that isolated force events or isolated acceleration events do not trigger detection of the defined oral event.

The system of paragraph [0191], wherein:
the processing subsystem is further configured to adapt at least one of the following of the defined oral event based on historical sensor data associated with the individual user:
   an occlusal force threshold;
   an acceleration threshold;
   a temporal correlation window; or
   the severity metric; and
the adaptation:
   is constrained by predefined safety limits and validated parameter ranges;
   is applied in real-time or near-real-time during system operation;
   improves event detection accuracy; and
   reduces false positive detections.

The system of paragraph [0194], wherein:
the adaptation is performed using at least one of:
   statistical analysis of historical event distributions;
   machine learning models;
   reinforcement-based adaptation; or
   rule-based adaptive threshold adjustment; and
the system reduces false positive detections and improves event classification accuracy over time.

The system of paragraph [0191], wherein the sensor fusion model evaluates:
temporal correlation between sensing modalities;
relative signal magnitude, and
pattern consistency across multiple detection cycles.

The system of paragraph [0191], wherein the intraoral body (110, 111, 112) comprises a dental guard, splint, mouthguard, orthodontic retainer form factor, or a custom shell configured for attachment to one or more teeth.

The system of paragraph [0191], wherein the intraoral body comprises a shell (111, 112) and a removable electronics module (1221, 1222, 1223), and wherein the removable electronics module is configured for insertion into and removal from the shell.

The system of paragraph [0198], wherein the removable electronics module (1221, 1222, 1223) is configured to be reusable across multiple shells (111, 112).

The system of paragraph [0191], wherein the sensing subsystem (200), processing subsystem (300, 310, 330), and intervention subsystem (400) are implemented as a single integrated electronics assembly.

The system of paragraph [0191], wherein the intraoral body (110, 111, 112) is provided in a plurality of predefined sizes and/or is configured to be user-specific.

The system of paragraph [0191], wherein the intraoral body (110, 111, 112) comprises:
a structural carrier portion for supporting electronic components; and
an encapsulation structure comprising biocompatible material configured to provide mechanical retention, sealing, and electrical insulation.

The system of paragraph [0191], wherein the at least one occlusal force sensing element (210, 211) comprises at least one of a piezoelectric element, piezoresistive element, strain gauge, capacitive force sensor, or force-sensitive resistor (210, 211).

The system of paragraph [0191], wherein the at least one inertial sensing element (230, 231, 232) comprises a multi-axis accelerometer (231), a multi-axis gyroscope (232), or a combined inertial measurement unit (230).

The system of paragraph [0191], wherein the sensing subsystem (200) further comprises at least one physiological indicator sensor (220, 221, 222, 240) configured to provide a trend-based, non-diagnostic physiological indicator correlated with stress, fatigue, or respiratory load.

The system of paragraph [0205], wherein the physiological indicator sensor (220) comprises at least one of an optical sensor (221, 222), impedance sensor, temperature sensor (240), or a proxy sensor configured to provide the trend-based, non-diagnostic physiological indicator.

The system of paragraph [0191], wherein the processing subsystem (300, 310, 330) determines the defined oral event based on thresholding of occlusal force and time-domain and/or statistical event features comprising at least one of event duration, event frequency, burst density, or inter-event timing.

The system of paragraph [0191], wherein:
the processing subsystem (300, 310, 330) classifies the defined oral event into at least two classes comprising a low-risk event and a high-risk event, based on at least one derived severity criterion; and
the severity criterion is derived from one or more parameters comprising occlusal force magnitude, event duration, repetition rate, burst density, motion intensity, or combinations thereof.

The system of paragraph [0191], wherein the vibration output comprises at least one of a micro-motor vibration output, a haptic actuator, or a piezoelectric vibration actuator (420, 421).

The system of paragraph [0191], wherein the neuromodulation stimulation subsystem output comprises at least one of biphasic electrical stimulation, pulsed electrical stimulation, or charge-balanced stimulation delivered via one or more electrodes (411, 412, 413, 414).

The system of paragraph [0191], wherein the neuromodulation stimulation subsystem output comprises biphasic, charge-balanced electrical pulses, and wherein the pulses comprise pulsed stimulation sequences configured to minimize DC offset and electrode polarization.

The system of paragraph [0191], wherein the escalation controller (900, 950) is configured to execute the first intervention stage using the vibration output and, upon persistence or escalation of the defined oral event, execute the second intervention stage using the neuromodulation stimulation subsystem output.

The system of paragraph [0191], wherein the neuromodulation stimulation subsystem (410) is generated within a stimulation domain (399) that is galvanically isolated from a low-voltage sensing and processing domain (391) and operates on an isolated supply and reference potential.

The system of paragraph [0191], further comprising at least one visual signal indicator (430) configured to present a device status while the intraoral body (110, 111, 112) is worn.

The system of paragraph [0214], wherein the visual signal indicator (430) is configured to present at least one of a normal status, caution status, or alert status based on the detected occurrence of the defined oral event and/or a computed severity level.

The system of paragraph [0191], wherein the safety subsystem (800) comprises a hardware current limiting circuit (810) configured to limit stimulation current to a maximum value independent of firmware.

The system of paragraph [0191], wherein the safety subsystem (800) comprises a DC-fault detection circuit (820) configured to detect a sustained DC offset condition in a stimulation path.

The system of paragraph [0191], wherein the safety subsystem (800) is configured such that, upon detection of a fault condition, the stimulation subsystem (410) is disabled by the fault-triggered latched stimulation disable state (840).

The system of paragraph [0218], wherein the safety subsystem (800) comprises a watchdog circuit (830) configured to trigger the fault-triggered latched stimulation disable state (840). if the processing subsystem fails to provide a defined heartbeat signal.

The system of paragraph [0191], further comprising a power management subsystem (500) comprising a rechargeable battery (510, 511) disposed within the intraoral body (110, 111, 112).

The system of paragraph [0220], wherein the power management subsystem (500) comprises wireless charging (600, 610, 620, 630).

The system of paragraph [0221], wherein the wireless charging (600) is disabled or limited while the intraoral body (110, 111, 112) is detected as being intraorally worn.

The system of paragraph [0191], wherein the processing subsystem (300, 310, 330) is configured to store event data comprising at least one of timestamps, severity score, peak force, or event duration.

The system of paragraph [0223], wherein the system is configured to transmit event data to an external device (1000) via a wireless communication link (320, 321).

The system of paragraph [0224], wherein the wireless communication link (320, 321) comprises Bluetooth Low Energy (321).

The system of paragraph [0191], wherein the system is configured to operate in an intraoral mode and a non-intraoral mode, and wherein the device intervention is inhibited in the non-intraoral mode.

The system of paragraph [0191], wherein the defined oral event comprises a bruxism event determined by repetitive occlusal force excursions above a bruxism threshold within a time window using the at least one occlusal force sensing element (210, 211).

The system of paragraph [0191], wherein the defined oral event comprises a clenching event determined by sustained occlusal force above a clenching threshold for a duration exceeding a time threshold using the at least one occlusal force sensing element (210, 211).

The system of paragraph [0191], wherein the defined oral event comprises an impact event determined by an acceleration threshold exceedance detected by the at least one inertial sensing element (230, 231, 232).

The system of paragraph [0191], wherein the defined oral event comprises a combined high-risk event determined based on:
simultaneous exceedance of an occlusal force threshold and an acceleration threshold;
a temporal correlation within a predefined time window, and
a consistency condition across multiple detection cycles.

The system of paragraph [0191], wherein the escalation controller (900, 950) selects an intervention stage based on at least one of event frequency, event intensity, or an accumulated dose metric.

The system of paragraph [0191], wherein the escalation controller (900, 950) selects an intervention stage based on an accumulated dose metric comprising a time-integrated measure of occlusal force exposure, event frequency, and/or delivered stimulation.

The system of paragraph [0191], wherein:
the escalation controller (900, 950) selects an intervention stage based on an accumulated dose metric comprising a cumulative measure of neuromodulation stimulation exposure over time comprising at least one of:
   total delivered charge;
   stimulation duration;
   stimulation intensity; or
   frequency of stimulation events; and
the escalation controller adjusts intervention stages based on the accumulated dose metric.

The system of paragraph [0191], wherein:
the system is configured to provide a recommendation prompt on an external application (1010) corresponding to an advisory device state; and
the advisory device state corresponds to a condition in which predefined thresholds are exceeded.

The system of paragraph [0191], wherein:
a standardized electronics module (117) is configured independently of user-specific geometry;
the intraoral body comprises predefined alignment and sealing interfaces configured to receive the standardized electronics module; and
the intraoral body comprises electrode exposure regions defined independent of the standardized electronics module.

The system of paragraph [0235], wherein the alignment and interface regions comprise one or more recesses (117) defining at least one of alignment features, retention features, connector access, electrode positioning constraints, or sealing interfaces, thereby enabling repeatable assembly across multiple intraoral bodies.

The system of paragraph [0191], wherein the intraoral body (110, 111, 112) is provided in a plurality of standardized shells (111, 112), and wherein a common electronics module (1221, 1222, 1223) is configured to be installed into each standardized shell.

The system of paragraph [0191], wherein the system comprises an application interface (1010, 1030) configured to display event summaries and device status on an external computing device (1000).

The system of paragraph [0191], wherein the processing subsystem (300, 310, 340) computes at least one bruxism-relevant metric selected from accumulated occlusal force exposure, peak-to-average ratio, event duration, event frequency, burst density, or right/left asymmetry, and wherein the escalation controller (900, 950) is triggered based on said metric.

The system of paragraph [0191], wherein the processing subsystem (300, 310, 340) assigns a severity score derived from at least two parameters comprising occlusal force magnitude and event repetition rate, and wherein an intervention stage is selected as a function of said severity score.

The system of paragraph [0191], wherein, after a selected intervention stage for the device intervention is executed, the escalation controller (900, 950) enforces a cooldown interval during which stimulation is inhibited or limited.

The system of paragraph [0241], wherein the cooldown interval comprises a lockout period during which escalation to a higher stage is disabled until at least one condition is met, comprising expiration of a timer, detection of reduced event activity, or removal of the intraoral body (110, 111, 112) from intraoral use.

The system of paragraph [0191], wherein the defined oral event comprises a combined high-risk event that is determined by sensor fusion requiring exceedance of an acceleration threshold and an occlusal force threshold within a defined time window, combined with a trend-based physiological indicator.

The system of paragraph [0191], wherein the defined oral event comprises a combined high-risk event that causes activation of an externally visible advisory status flag (430, 431, 432) while the intraoral body (110, 111, 112) remains intraorally worn.

The system of paragraph [0244], wherein the externally visible advisory status flag (430) is configured to be recognized by a third party without access to an external application (1010) comprising a coach, trainer, referee, supervisor, or caregiver, and wherein the advisory status flag is non-diagnostic and indicates that a predefined device threshold has been exceeded.

The system of paragraph [0191], wherein:
a standardized electronics module (117) comprising the sensing subsystem (200), the processing subsystem (300), a power management subsystem (500), and the intervention subsystem (400) is identical across multiple device variants;
the intraoral body (110, 111, 112) is configured using a computer-aided design (“CAD”) workflow that defines one or more placeholders or recesses (117) to receive at least one standardized electronics module (1221, 1222, 1223); and
user-specific adaptation is achieved by integrating said standardized electronics module into user-specific shells using the CAD-defined placeholders.

The system of paragraph [0246], wherein the at least one standardized electronics module (1221, 1222, 1223) is configured to be identical across at least three shell variants comprising a user-specific shell, a first standardized size shell, and a second standardized size shell.

The system of paragraph [0191], wherein the system is configured to detect an impact-related event while the intraoral body (110, 111, 112) is worn, and to transition the intraoral body (110, 111, 112) into an advisory state when at least one impact-related threshold is exceeded.

The system of paragraph [0248], wherein the impact-related event comprises a combined high-risk event determined by exceedance of an acceleration threshold and an occlusal force threshold within a defined time window, and wherein said defined time window is less than 250 milliseconds.

The system of paragraph [0248], wherein the advisory state activates a visual signal indicator (430) configured to present a severity pattern comprising at least one of: steady illumination, pulsed illumination, multi-level blinking, or a color-coded pattern.

The system of paragraph [0248], wherein the advisory state is externally visible to a third party while the intraoral body (110, 111, 112) remains intraorally worn, and wherein the third party comprises at least one of a coach, trainer, referee, supervisor, caregiver, or medical staff.

The system of paragraph [0248], wherein the advisory state causes at least one protective response comprising at least one of: inhibiting escalation to a higher stimulation stage, disabling stimulation output, enforcing a lockout interval, or requiring a deliberate reset condition prior to resuming stimulation.

The system of paragraph [0248], wherein the processing subsystem (300, 310, 330) stores a post-event record associated with the impact-related event comprising at least one of: timestamp, peak acceleration, peak occlusal force, event duration, severity score, or device intervention state.

The system of paragraph [0191], wherein the detected occurrence of the defined oral event triggers a protective device state comprising:
inhibition of neuromodulation stimulation;
enforcement of a lockout interval, and
generation of an advisory signal.

The system of paragraph [0254], wherein the advisory signal is configured as a non-diagnostic indicator representing that a predefined threshold has been exceeded.

The system of paragraph [0191], wherein electrode placement of the intraoral body is configured along the dental arch in proximity to masticatory muscle insertion regions when the intraoral body is worn within the oral cavity.

The system of paragraph [0256], wherein stimulation selectivity is achieved through electrode geometry, spacing, and controlled current distribution of the system.

The system of paragraph [0191], wherein neuromodulation stimulation is enabled only when a valid intraoral wear-state is detected.

The system of paragraph [0258], wherein the intraoral wear-state is determined using at least one of:
impedance measurement;
temperature sensing;
motion pattern recognition; or
capacitive proximity detection.

The system of paragraph [0191], wherein stimulation is inhibited during charging of an internal energy storage element.

The system of paragraph [0191], wherein the escalation controller enforces a cooldown interval following neuromodulation stimulation.

The system of paragraph [0191], wherein the system is configured to modify intervention parameters based on detected long-term behavioral trends indicative of neuromuscular adaptation.

The system of paragraph [0191], wherein conflicting sensor signals comprise inconsistent or non-correlated signals between occlusal force and inertial motion parameters, and wherein the processing subsystem suppresses or inhibits event detection based on the conflicting sensor signals.

An intraoral neuromodulation safety architecture, comprising:
(a) a stimulation subsystem (410) configured to generate charge-balanced stimulation pulses;
(b) an isolation barrier (720) separating the stimulation subsystem from a sensing and control subsystem;
(c) a hardware safety enforcement subsystem (800) comprising:
   (c1) a primary current limiting circuit (810);
   (c2) an independent secondary current limiting circuit;
   (c3) a DC-offset detection circuit (820) configured to detect sustained electrode polarization; and
   (c4) a watchdog circuit (830) configured to detect absence of a valid control signal; and
(d) a fault latch (840) configured to force the stimulation subsystem into a disabled state upon detection of any unsafe condition, wherein:
   stimulation defaults to an off-state upon power-up or reset;
   net delivered charge is limited;
   mean electrode potential remains within a predefined safe range; and
   stimulation remains disabled until a hardware-validated reset condition is satisfied.

The safety architecture of paragraph [0264], wherein the hardware safety enforcement subsystem (800) prevents any firmware command, external command, or wireless command from increasing stimulation energy beyond the enforced stimulation output constraints.

The safety architecture of paragraph [0264], wherein the hardware-validated reset condition involves a physical or hardware-detected action not achievable solely by software.

The safety architecture of paragraph [0264], wherein the hardware safety enforcement subsystem (800) is configured to enforce stimulation output constraints.

The safety architecture of paragraph [0267], wherein the stimulation output constraints comprise limits for current, voltage, pulse width, repetition rate, burst duration, and duty cycle enforced by hardware circuits.

The safety architecture of paragraph [0267], wherein the stimulation output constraints are enforced by hardware and/or hardware-assisted digital circuits.

The safety architecture of paragraph [0267], wherein the stimulation output constraints comprise at least one of: a maximum stimulation current, a maximum pulse width, a maximum pulse repetition rate, a maximum burst duration, or a maximum stimulation duty cycle.

The safety architecture of paragraph [0264], wherein the isolation barrier (720) defines a low-voltage domain (391) comprising the sensing and control subsystem (200, 300, 500) and an isolated stimulation domain (399) comprising the stimulation subsystem (410, 700), and wherein the stimulation domain (399) operates on an isolated supply and an isolated reference potential.

The safety architecture of paragraph [0271], wherein the isolated supply is provided by transformer-based isolation or capacitive isolation.

The safety architecture of paragraph [0264], wherein each one of the primary current limiting circuit and the secondary current limiting circuit is configured to trigger the disabled state upon detection of overcurrent.

The safety architecture of paragraph [0264], wherein the DC-offset detection circuit (820) is configured to detect a sustained DC offset indicative of electrode polarization or capacitor polarization, and wherein the detection of the sustained DC offset triggers the disabled state.

The safety architecture of paragraph [0274], wherein a DC-blocking capacitor (750) is disposed in series with at least one stimulation electrode path, and wherein the DC-offset detection circuit (820) is configured to monitor at least one of a voltage across the DC-blocking capacitor (750) and a mean electrode potential to determine the sustained DC offset.

The safety architecture of paragraph [0264], wherein the watchdog circuit (830) is configured to detect a nonresponsive control condition based on absence of a heartbeat signal or presence of an invalid timing sequence, and wherein the watchdog circuit (830) triggers the disabled state.

The safety architecture of paragraph [0264], wherein stimulation is enabled only when at least two independent safety conditions are satisfied.

A platform architecture for intraoral sensing and intervention comprising:
an intraoral interface structure (110, 111, 112) configured to engage teeth;
a sensing subsystem (200);
an event detection subsystem (300) configured to detect events based on a combined evaluation of occlusal force and inertial motion parameters sensed by the sensing subsystem within a predefined temporal window;
an intervention subsystem (400) comprising at least two feedback modalities comprising mechanical feedback and neuromodulation; and
a safety subsystem (800) configured to enforce hardware-defined operational limits independent of software execution.

The platform architecture of paragraph [0278], wherein additional sensing modules or intervention modules are integratable into the platform without modification of the core platform architecture.

The platform architecture of paragraph [0278], wherein the sensing subsystem (200) comprises at least one force sensor and at least one inertial sensor.

The platform architecture of paragraph [0278], wherein the intervention subsystem (400) comprises one or more outputs selected from the group consisting of vibration outputs, electrical stimulation outputs, optical outputs, acoustic outputs, or combinations thereof.

One, some, or all of the processes described herein may each be partially or entirely implemented by software, but may also be implemented in hardware, firmware, or any combination of software, hardware, and firmware. Instructions for performing these processes may also be embodied as machine-or computer-readable code recorded on a machine-or computer-readable medium. In some embodiments, the computer-readable medium may be a non-transitory computer-readable medium. Examples of such a non-transitory computer-readable medium include but are not limited to a read-only memory, a random-access memory, a flash memory, a CD-ROM, a DVD, a magnetic tape, a removable memory card, and a data storage device (e.g., memory 13 of FIG. 1A). In other embodiments, the computer-readable medium may be a transitory computer-readable medium. In such embodiments, the transitory computer-readable medium can be distributed over network-coupled computer systems so that the computer-readable code is stored and executed in a distributed fashion. For example, such a transitory computer-readable medium may be communicated from a BMS subsystem to a monitoring subsystem, from a monitoring subsystem to a BMS subsystem, between a monitoring subsystem and an intraoral apparatus, and/or from any suitable subsystem to any other suitable subsystem using any suitable communications protocol (e.g., the computer-readable medium may be communicated to a subsystem as at least a portion of an application 19 via any suitable communications components and/or via any suitable network(s) 101). Such a transitory computer-readable medium may embody computer-readable code, instructions, data structures, program modules, or other data in a modulated data signal, such as a carrier wave or other transport mechanism, and may include any information delivery media. A modulated data signal may be a signal that has one or more of its characteristics set or changed in such a manner as to encode information in the signal.

Any, each, or at least one module or component or subsystem of the disclosure may be provided as a software construct, firmware construct, one or more hardware components, or a combination thereof. For example, any, each, or at least one module or component or subsystem of system 1 may be described in the general context of computer-executable instructions, such as program modules, that may be executed by one or more computers or other devices. Generally, a program module may include one or more routines, programs, objects, components, and/or data structures that may perform one or more particular tasks or that may implement one or more particular abstract data types. The number, configuration, functionality, and interconnection of the modules and components and subsystems of system 1 are only illustrative, and that the number, configuration, functionality, and interconnection of existing modules, components, and/or subsystems may be modified or omitted, additional modules, components, and/or subsystems may be added, and the interconnection of certain modules, components, and/or subsystems may be altered.

Implementations within the scope of the present disclosure can be partially or entirely realized using a tangible computer-readable storage medium, or multiple tangible computer-readable storage media of one or more types, encoding one or more instructions. The tangible computer-readable storage medium also can be non-transitory in nature.

At least a portion of one or more of the modules of system 1 may be stored in or otherwise accessible to a subsystem (e.g., subsystem 108) in any suitable manner (e.g., in memory assembly 13 (e.g., as at least a portion of application 19 (e.g., application 19a))). Any or each module of system 1 may be implemented using any suitable technologies (e.g., as one or more integrated circuit devices), and different modules may or may not be identical in structure, capabilities, and operation. Any or all of the modules or other components of system 1 may be mounted on an expansion card, mounted directly on a system motherboard, or integrated into a system chipset component (e.g., into a “north bridge” chip).

Any or each module of system 1 may be a dedicated system implemented using one or more expansion cards adapted for various bus standards. For example, all of the modules may be mounted on different interconnected expansion cards or all of the modules may be mounted on one expansion card. With respect to system 1, by way of example only, the modules of system 1 may interface with a motherboard or processor assembly 12 (e.g., of subsystem 108) through an expansion slot (e.g., a peripheral component interconnect (“PCI”) slot or a PCI express slot).

Alternatively, a module need not be removable but may include one or more dedicated modules that may include memory (e.g., RAM) dedicated to the utilization of the module. Any or each module may include its own processing circuitry and/or memory. Alternatively, any or each module may share processing circuitry and/or memory with any other module and/or processor assembly 12 and/or memory assembly 13 of the subsystem (e.g., subsystem 108).

The computer-readable storage medium can be any storage medium that can be read, written, or otherwise accessed by a general purpose or special purpose computing device, including any processing electronics and/or processing circuitry capable of executing instructions. For example, without limitation, the computer-readable medium can include any volatile semiconductor memory, such as RAM, DRAM, SRAM, T-RAM, Z-RAM, and TTRAM. The computer-readable medium also can include any non-volatile semiconductor memory, such as ROM, PROM, EPROM, EEPROM, NVRAM, flash, nvSRAM, FeRAM, FeTRAM, MRAM, PRAM, CBRAM, SONOS, RRAM, NRAM, racetrack memory, FJG, and Millipede memory.

Further, the computer-readable storage medium can include any non-semiconductor memory, such as optical disk storage, magnetic disk storage, magnetic tape, other magnetic storage devices, or any other medium capable of storing one or more instructions. In one or more implementations, the tangible computer-readable storage medium can be directly coupled to a computing device, while in other implementations, the tangible computer-readable storage medium can be indirectly coupled to a computing device (e.g., via one or more wired connections, one or more wireless connections, or any combination thereof).

Instructions can be directly executable or can be used to develop executable instructions. For example, instructions can be realized as executable or non-executable machine code or as instructions in a high-level language that can be compiled to produce executable or non-executable machine code. Further, instructions also can be realized as or can include data.

Computer-executable instructions also can be organized in any format, including, but not limited to, routines, subroutines, programs, data structures, objects, modules, applications, applets, functions, and/or the like. As recognized by those of skill in the art, details including, but not limited to, the number, structure, sequence, and organization of instructions can vary significantly without varying the underlying logic, function, processing, and output.

While the above discussion primarily refers to microprocessor or multi-core processors that execute software, one or more implementations may be performed by one or more integrated circuits, such as ASICs or FPGAs. In one or more implementations, such integrated circuits may execute instructions that may be stored on the circuit itself.

Those of skill in the art would appreciate that the various illustrative blocks, modules, elements, components, methods, and algorithms described herein may be implemented as electronic hardware, computer software, or combinations of both. To illustrate this interchangeability of hardware and software, various illustrative blocks, modules, elements, components, methods, and algorithms have been described above generally in terms of their functionality. Whether such functionality is implemented as hardware or software may depend upon the particular application and design constraints imposed on the overall system. Skilled artisans may implement the described functionality in varying ways for each particular application. Various components and blocks may be arranged differently (e.g., arranged in a different order, or partitioned in a different way) all without departing from the scope of the subject technology.

It is understood that any specific order or hierarchy of blocks in the processes disclosed is an illustration of example approaches. Based upon design preferences, it is understood that the specific order or hierarchy of blocks in the processes may be rearranged, or that all illustrated blocks be performed. Any of the blocks may be performed simultaneously. In one or more implementations, multitasking and parallel processing may be advantageous. Moreover, the separation of various system components in the implementations described above should not be understood as requiring such separation in all implementations, and it should be understood that the described program components and systems can generally be integrated together in a single software product or packaged into multiple software products.

Any suitable system model (e.g., model 19m) may be developed and/or generated for use in evaluating and/or predicting output states. For example, a model may be a learning engine for an experiencing entity, where the learning engine may be operative to use any suitable machine learning (“ML”) (e.g., the system's ability to learn automatically from past events to affect future behavior) to use certain monitored system data for a particular environment (e.g., at a particular time and/or with respect to one or more planned activities) in order to predict, estimate, and/or otherwise generate an output state. For example, the learning engine may include any suitable neural network (e.g., an artificial neural network) that may be initially configured, trained on one or more sets of monitored system data that is associated with known or otherwise determined or confirmed states or data from any suitable sources, and then used to predict further states based on another set of monitored system data.

A neural network or neuronal network or artificial neural network may be hardware-based, software-based, or any combination thereof, such as any suitable model (e.g., an analytical model, a computational model, etc.), which, in some embodiments, may include one or more sets or matrices of weights (e.g., adaptive weights, which may be numerical parameters that may be tuned by one or more learning algorithms or training methods or other suitable processes) and/or may be capable of approximating one or more functions (e.g., non-linear functions or transfer functions) of its inputs. The weights may be connection strengths between neurons of the network, which may be activated during training and/or prediction. A neural network may generally be a system of interconnected neurons that can compute values from inputs and/or that may be capable of machine learning and/or pattern recognition (e.g., due to an adaptive nature). A neural network may use any suitable machine learning techniques to optimize a training process. The neural network may be used to estimate or approximate functions that can depend on a large number of inputs and that may be generally unknown. The neural network may generally be a system of interconnected “neurons” that may exchange messages between each other, where the connections may have numeric weights (e.g., initially configured with initial weight values) that can be tuned based on experience, making the neural network adaptive to inputs and capable of learning (e.g., learning pattern recognition). A suitable optimization or training process may be operative to modify a set of initially configured weights assigned to the output of one, some, or all neurons from the input(s) and/or hidden layer(s). A non-linear transfer function may be used to couple any two portions of any two layers of neurons, including an input layer, one or more hidden layers, and an output (e.g., an input to a hidden layer, a hidden layer to an output, etc.).

Different input neurons of the neural network may be associated with respective different types of monitored system data categories and may be activated by monitored system data of the respective monitored system data categories (e.g., each possible category of monitored system data variable information may be associated with one or more particular respective input neurons of the neural network and monitored system data for the particular monitored system data category may be operative to activate the associated input neuron(s)). The weight assigned to the output of each neuron may be initially configured using any suitable determinations that may be made by a custodian or processor of the model based on the data available to that custodian.

The initial configuring of the learning engine or management model for a particular system (e.g., the initial weighting and arranging of neurons of a neural network of the learning engine) may be done using any suitable data accessible to a custodian of the management model, such as data associated with the configuration of other learning engines of the system (e.g., learning engines or management models for other systems), data associated with the particular system (e.g., initial background data accessible by the model custodian about the particular system composition, location, past uses, and/or the like), data assumed or inferred by the model custodian using any suitable guidance, and/or the like. For example, a model custodian may be operative to capture any suitable initial background data about a particular system in any suitable manner, which may be enabled by any suitable user interface provided to an appropriate subsystem or device accessible to one, some, or each operator or entity with knowledge of the particular system (e.g., a model app or website). The model custodian may provide a data collection portal for enabling any suitable entity to provide initial background data for the particular system. The data may be uploaded in bulk or manually entered in any suitable manner.

A management model custodian may receive not only monitored system data for at least one monitored system data category for a particular system experience but also a system output product state for that system experience. This may be enabled by monitoring any suitable system data for a system. The management model custodian may provide a data collection portal for enabling any suitable entity(ies) to provide such data. The system output state may be received and may be derived from the system in any suitable manner.

A learning engine or model for a system may be using the received monitored system data for the system experience (e.g., as inputs of a neural network of the learning engine) and using the received system output product state for the system experience (e.g., as an output of the neural network of the learning engine). Any suitable training methods or algorithms (e.g., learning algorithms) may be used to train the neural network of the learning engine, including, but not limited to, Back Propagation, Resilient Propagation, Genetic Algorithms, Simulated Annealing, Levenberg, Nelder-Meade, and/or the like. Such training methods may be used individually and/or in different combinations to get the best performance from a neural network. A loop (e.g., a receipt and train loop) of receiving monitored system data and a system output product state for a system experience (e.g., a particular system in a particular environment at a particular moment) and then training the system model using the received monitored system data and system output product state may be repeated any suitable number of times for the same system(s) in different system experiences (e.g., in same or different environments at different moments) and the same learning engine for more effectively training the learning engine for the system, where the received monitored system data and the received system output product state of different receipt and train loops may be for different environments or for the same environment (e.g., at different times and/or with respect to different planned activities) and/or may be received from the same source or from different sources of the system, while the training of different receipt and train loops may be done for the same learning engine using whatever monitored system data and system output product state was received for the particular receipt and train loop. The number and/or type(s) of the one or more monitored system data categories for which monitored system data may be received for one receipt and train loop may be the same or different in any way(s) than the number and/or type(s) of the one or more monitored system data categories for which monitored system data may be received for a second receipt and train loop.

A trained model may then receive input data from any suitable source using any suitable methods for use by the model. The trained model may then use this new input data to generate output data using the learning engine or model. For example, the new input data may be utilized as input(s) to the neural network of the learning engine similarly to how other input data accessed for a receipt and train loop may be utilized as input(s) to the neural network of the learning engine at a training portion of the receipt and train loop, and such utilization of the learning engine with respect to the new input data may result in the neural network providing an output indicative of data that may represent the learning engine's predicted or estimated result.

The processing power and speed of any suitable BMSP and its various models may be configured to determine continuously an updated system output product state of a system and present associated information or otherwise adjust a managed element based on the determined system output product state automatically and instantaneously or substantially instantaneously based on any new received monitored system data that may be generated by the system, such that management of the system may run quickly and smoothly. This may enable the system to operate as effectively and as efficiently as possible.

The use of one or more suitable models or engines or neural networks or the like may enable prediction or any suitable determination of an output product state of a system in a system experience. Such models (e.g., neural networks) running on any suitable processing units (e.g., graphical processing units (“GPUs”) that may be available to the system) provide significant speed improvements in efficiency and accuracy with respect to prediction over other types of algorithms and human-conducted analysis of data, as such models can provide estimates in a few milliseconds or less, thereby improving the functionality of any computing device on which they may be run. Due to such efficiency and accuracy, such models enable a technical solution for enabling the generation of any suitable control data (e.g., for controlling any suitable functionality of any suitable managed element) using any suitable real-time data (e.g., data made available to the models) that may not be possible without the use of such models, as such models may increase performance of their computing device(s) by requiring less memory, providing faster response times, and/or increased accuracy and/or reliability. Due to the condensed time frame and/or the time within which a decision with respect to system data ought to be made to provide a desirable use experience, such models offer the unique ability to provide accurate determinations with the speed necessary to enable effective and efficient use management (e.g., management of bruxism).

As used in this specification and any claims of this application, the terms “base station,” “receiver,” “computer,” “server,” “processor,” and “memory” may all refer to electronic or other technological devices. These terms exclude people or groups of people. For the purposes of the specification, the terms “display” or “displaying” means displaying on an electronic device.

The terminology used in the description of the various described embodiments herein is for the purpose of describing particular embodiments only and is not intended to be limiting. As used in the description of the various described embodiments and the appended claims, the singular forms “a,” “an,” and “the” are intended to include the plural forms as well, unless the context clearly indicates otherwise. The term “and/or” as used herein refers to and encompasses any and all possible combinations of one or more of the associated listed items. As used herein, the phrase “at least one of” preceding a series of items, with the term “and” or “or” to separate any of the items, modifies the list as a whole, rather than each member of the list (i.e., each item). The phrase “at least one of” does not require selection of at least one of each item listed; rather, the phrase allows a meaning that includes at least one of any one of the items, and/or at least one of any combination of the items, and/or at least one of each of the items. By way of example, the phrases “at least one of A, B, and C” or “at least one of A, B, or C” may each refer to only A, only B, or only C; any combination of A, B, and C; and/or at least one of each of A, B, and C. The terms “includes,” “including,” “comprises,” and/or “comprising,” when used in this specification, specify the presence of stated features, integers, steps, operations, elements, and/or components, but do not preclude the presence or addition of one or more other features, integers, steps, operations, elements, components, and/or groups thereof.

The term “if” is, optionally, construed to mean “when” or “upon” or “in response to determining” or “in response to detecting,” depending on the context. Similarly, the phrase “if it is determined” or “if [a stated condition or event] is detected” is, optionally, construed to mean “upon determining” or “in response to determining” or “upon detecting [the stated condition or event]” or “in response to detecting [the stated condition or event],” depending on the context.

As used herein, the terms “computer,” “personal computer,” “device,” and “computing device” may refer to any programmable computer system that is known or that will be developed in the future. In certain embodiments, a computer will be coupled to a network, such as described herein. A computer system may be configured with processor-executable software instructions to perform the processes described herein. Such computing devices may be mobile devices, such as a mobile telephone, data assistant, tablet computer, or other such mobile devices. Alternatively, such computing devices may not be mobile (e.g., in at least certain use cases), such as in the case of server computers, desktop computing systems, or systems integrated with non-mobile components.

As used herein, the terms “component,” “module,” and “system,” are intended to refer to a computer-related entity, either hardware, a combination of hardware and software, software, or software in execution. For example, a component may be, but is not limited to being, a process running on a processor, a processor, an object, an executable, a thread of execution, a program, and/or a computer. By way of illustration, both an application running on a server and the server may be a component. One or more components may reside within a process and/or thread of execution and a component may be localized on one computer and/or distributed between two or more computers.

The predicate words “configured to,” “operable to,” and “programmed to” do not imply any particular tangible or intangible modification of a subject, but, rather, are intended to be used interchangeably. In one or more implementations, a processor configured to monitor and control an operation or a component may also mean the processor being programmed to monitor and control the operation or the processor being operable to monitor and control the operation. Likewise, a processor configured to execute code can be construed as a processor programmed to execute code or operable to execute code.

Phrases such as an aspect, the aspect, another aspect, some aspects, one or more aspects, an implementation, the implementation, another implementation, some implementations, one or more implementations, an embodiment, the embodiment, another embodiment, some implementations, one or more implementations, a configuration, the configuration, another configuration, some configurations, one or more configurations, the subject technology, the disclosure, the present disclosure, other variations thereof and alike are for convenience and do not imply that a disclosure relating to such phrase(s) is essential to the subject technology or that such disclosure applies to all configurations of the subject technology. A disclosure relating to such phrase(s) may apply to all configurations, or one or more configurations. A disclosure relating to such phrase(s) may provide one or more examples. A phrase such as an aspect or some aspects may refer to one or more aspects and vice versa, and this applies similarly to other foregoing phrases.

The word “exemplary” is used herein to mean “serving as an example, instance, or illustration”. Any embodiment described herein as “exemplary” or as an “example” is not necessarily to be construed as preferred or advantageous over other implementations. Furthermore, to the extent that the term “include,” “have,” or the like is used in the description or the claims, such term is intended to be inclusive in a manner similar to the term “comprise” as “comprise” is interpreted when employed as a transitional word in a claim.

All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112, sixth paragraph, unless the element is expressly recited using the phrase “means for” or, in the case of a method claim, the element is recited using the phrase “step for”.

The previous description is provided to enable any person skilled in the art to practice the various aspects described herein. Various modifications to these aspects will be readily apparent to those skilled in the art, and the generic principles defined herein may be applied to other aspects. Thus, the claims are not intended to be limited to the aspects shown herein, but are to be accorded the full scope consistent with the language claims, wherein reference to an element in the singular is not intended to mean “one and only one” unless specifically so stated, but rather “one or more”. Unless specifically stated otherwise, the term “some” refers to one or more. Pronouns in the masculine (e.g., his) include the feminine and neuter/neutral gender (e.g., her and its and they) and vice versa. Headings and subheadings, if any, are used for convenience only and do not limit the subject disclosure.

One aspect of the present technology may be the gathering and use of data available from various sources to improve the management of bruxism and user benefits thereof. The present disclosure contemplates that in some instances, this gathered data may include personal information data that uniquely identifies or can be used to contact or locate a specific person. Such personal information data can include demographic data, location-based data, telephone numbers, email addresses, social network identifiers, home addresses, office addresses, data or records relating to a user's health or level of fitness (e.g., vital signs measurements, facial expression measurements, medication information, exercise information, etc.) and/or mindfulness, date of birth, or any other identifying or personal information.

The present disclosure recognizes that the use of such personal information data, in the present technology, can be used to the benefit of users. For example, the personal information data can be used to improve the determination of user profiles and management of bruxism.

Further, other uses for personal information data that benefit the user are also contemplated by the present disclosure. For instance, health and fitness data may be used to provide insights into a user's general wellness, or may be used as positive feedback to individuals using technology to pursue wellness goals.

The present disclosure contemplates that the entities responsible for the collection, analysis, disclosure, transfer, storage, or other use of such personal information data will comply with well-established privacy policies and/or privacy practices. In particular, such entities should implement and consistently use privacy policies and practices that are generally recognized as meeting or exceeding industry or governmental requirements for maintaining personal information data private and secure. Such policies should be easily accessible by users, and should be updated as the collection and/or use of data changes. Personal information from users should be collected for legitimate and reasonable uses of the entity and not shared or sold outside of those legitimate uses. Further, such collection/sharing should occur after receiving the informed consent of the users. Additionally, such entities should consider taking any needed steps for safeguarding and securing access to such personal information data and ensuring that others with access to the personal information data adhere to their privacy policies and procedures. Further, such entities can subject themselves to evaluation by third parties to certify their adherence to widely accepted privacy policies and practices. In addition, policies and practices should be adapted for the particular types of personal information data being collected and/or accessed and adapted to applicable laws and standards, including jurisdiction-specific considerations. For instance, in the United States, collection of or access to certain health data may be governed by federal and/or state laws, such as the Health Insurance Portability and Accountability Act (“HIPAA”); whereas health data in other countries may be subject to other regulations and policies and should be handled accordingly. Hence different privacy practices should be maintained for different personal data types in each country.

Despite the foregoing, the present disclosure also contemplates embodiments in which users selectively block the use of, or access to, personal information data. That is, the present disclosure contemplates that hardware and/or software elements can be provided to prevent or block access to such personal information data. For example, in the case of location detection services, the present technology can be configured to allow users to select to “opt in” or “opt out” of participation in the collection of personal information data during registration for services or anytime thereafter. In addition to providing “opt in” or “opt out” options, the present disclosure contemplates providing notifications relating to the access or use of personal information. For instance, a user may be notified upon downloading an app that their personal information data will be accessed and then reminded again just before personal information data is accessed by the app.

Moreover, it is the intent of the present disclosure that personal information data should be managed and handled in a way to minimize risks of unintentional or unauthorized access or use. Risk can be minimized by limiting the collection of data and deleting data once it is no longer needed. In addition, and when applicable, including in certain health related applications, data de-identification can be used to protect a user's privacy. De-identification may be facilitated, when appropriate, by removing specific identifiers (e.g., date of birth, etc.), controlling the amount or specificity of data stored (e.g., collecting location data a city level rather than at an address level), controlling how data is stored (e.g., aggregating data across users), and/or other methods.

Therefore, although the present disclosure broadly covers use of personal information data to implement one or more various disclosed embodiments, the present disclosure also contemplates that the various embodiments can also be implemented without the need for accessing such personal information data. That is, the various embodiments of the present technology are not rendered inoperable due to the lack of all or a portion of such personal information data. For example, the determination of user profiles and/or management of bruxism can be made based on non-personal information data or a bare minimum amount of personal information, such as the content being requested by the device associated with a user, other non-personal information available to the device, or publicly available information.

It is also to be understood that various directional and orientational terms, such as “up” and “down,” “left” and “right,” “forward” and “back,” “edge” and “corner,” “top” and “bottom” and “side,” “above” and “below,” “length” and “width” and “thickness” and “diameter” and “cross-section” and “longitudinal,” “X-” and “Y-” and “Z-,” “roll” and “pitch” and “yaw,” “clockwise” and “counter-clockwise,” and/or the like, may be used herein only for convenience, and that no fixed or absolute directional or orientational limitations are intended by the use of these terms. For example, the components of the system can have any desired orientation. If reoriented, different directional or orientational terms may need to be used in their description, but that will not alter their fundamental nature as within the scope and spirit of the subject matter described herein in any way.

While there have been described systems, methods, and computer-readable media for managing bruxism, many changes may be made therein without departing from the spirit and scope of the subject matter described herein in any way. Insubstantial changes from the claimed subject matter as viewed by a person with ordinary skill in the art, now known or later devised, are expressly contemplated as being equivalently within the scope of the claims. Therefore, obvious substitutions now or later known to one with ordinary skill in the art are defined to be within the scope of the defined elements.

Therefore, those skilled in the art will appreciate that the concepts of the disclosure can be practiced by other than the described embodiments, which are presented for purposes of illustration rather than of limitation.

1. An intraoral neuromodulation system (100), comprising:
(a) a tooth-supported or dental-arch supported intraoral body (110, 111, 112) configured to be worn within an oral cavity of an individual user;
(b) a sensing subsystem (200) integrated within the intraoral body and comprising:
at least one occlusal force sensing element (210, 211) configured to detect occlusal loading; and
at least one inertial sensing element (230, 231, 232) configured to detect motion parameters comprising at least one of acceleration or angular velocity;
(c) a processing subsystem (300, 310, 330) configured to:
receive sensor signals from the sensing subsystem; and
detect an occurrence of a defined oral event based on a combined evaluation of occlusal loading and motion parameters of the received sensor signals within a predefined temporal window, wherein the defined oral event comprises a dynamically classified oral event based on multi-parameter sensor fusion;
(d) an intervention subsystem (400) configured to execute a device intervention in response to the detected occurrence of the defined oral event, the intervention subsystem comprising:
a vibration output (420, 421) ; and
a neuromodulation stimulation subsystem output (410) configured to alter neuromuscular activity without inducing a direct muscle contraction;
(e) an escalation controller (900, 950) configured to select between a plurality of intervention stages for the device intervention based on a severity metric derived from at least occlusal force magnitude and motion intensity, wherein the plurality of intervention stages comprises at least a first stage (950b) using the vibration output and a second stage (950c) using the neuromodulation stimulation subsystem output; and
(f) a safety subsystem (800) configured to:
enforce stimulation output constraints using hardware-defined safety limits that are independent of the processing subsystem and not overrideable by firmware; and
maintain a default-off stimulation state and require validation of multiple independent conditions prior to enabling stimulation, wherein:
the hardware-defined safety limits comprise at least a hard current limit (810) and a fault-triggered latched stimulation disable state (840), and
the defined oral event is determined using a multi-parameter sensor fusion model.

2. (canceled)

3. The system of claim 1, wherein the defined oral event requires:
exceedance of an occlusal force threshold;
exceedance of an acceleration threshold, and
a temporal coincidence within less than 300 milliseconds, such that isolated force events or isolated acceleration events do not trigger detection of the defined oral event.

4. The system of claim 1, wherein:
the processing subsystem is further configured to adapt at least one of the following of the defined oral event based on historical sensor data associated with the individual user:
an occlusal force threshold;
an acceleration threshold;
a temporal correlation window; or
the severity metric; and
the adaptation:
is constrained by predefined safety limits and validated parameter ranges;
is applied in real-time or near-real-time during system operation;
improves event detection accuracy; and
reduces false positive detections.

5. (canceled)

6. The system of claim 1, wherein the sensor fusion model evaluates:
temporal correlation between sensing modalities;
relative signal magnitude, and
pattern consistency across multiple detection cycles.

7. (canceled)

8. The system of claim 1, wherein the intraoral body comprises a shell (111, 112) and a removable electronics module (1221, 1222, 1223), and wherein the removable electronics module is configured for insertion into and removal from the shell.

9. The system of claim 8, wherein the removable electronics module (1221, 1222, 1223) is configured to be reusable across multiple shells (111, 112).

10. The system of claim 1, wherein the sensing subsystem (200), processing subsystem (300, 310, 330), and intervention subsystem (400) are implemented as a single integrated electronics assembly.

11-22. (canceled)

23. The system of claim 1, wherein the neuromodulation Stimulation subsystem (410) is generated within a stimulation domain (399) that is galvanically isolated from a low-voltage sensing and processing domain (391) and operates on an isolated supply and reference potential.

24. The system of claim 1, further comprising at least one visual signal indicator (430) configured to present a device status while the intraoral body (110, 111, 112) is worn.

25. The system of claim 24, wherein the visual signal indicator (430) is configured to present at least one of a normal status, caution status, or alert status based on the detected occurrence of the defined oral event and/or a computed severity level.

26-53. (canceled)

54. The system of claim 1, wherein the defined oral event comprises a combined high-risk event that causes activation of an externally visible advisory status flag (430, 431, 432) while the intraoral body (110, 111, 112) remains intraorally worn.

55-63. (canceled)

64. The system of claim 1, wherein the detected occurrence of the defined oral event triggers a protective device state comprising:
inhibition of neuromodulation stimulation;
enforcement of a lockout interval, and
generation of an advisory signal.

65-73. (canceled)

74. An intraoral neuromodulation safety architecture, comprising:
(a) a stimulation subsystem (410) configured to generate charge-balanced stimulation pulses;
(b) an isolation barrier (720) separating the stimulation subsystem from a sensing and control subsystem;
(c) a hardware safety enforcement subsystem (800) comprising:
(c1) a primary current limiting circuit (810) ;
(c2) an independent secondary current limiting circuit;
(c3) a DC-offset detection circuit (820) configured to detect sustained electrode polarization; and
(c4) a watchdog circuit (830) configured to detect absence of a valid control signal; and
(d) a fault latch (840) configured to force the stimulation subsystem into a disabled state upon detection of any unsafe condition, wherein:
stimulation defaults to an off-state upon power-up or reset;
net delivered charge is limited;
mean electrode potential remains within a predefined safe range; and
stimulation remains disabled until a hardware-validated reset condition is satisfied.

75. (canceled)

76. (canceled)

77. The safety architecture of claim 74, wherein the hardware safety enforcement subsystem (800) is configured to enforce stimulation output constraints.

78. The safety architecture of claim 77, wherein the stimulation output constraints comprise limits for current, voltage, pulse width, repetition rate, burst duration, and duty cycle enforced by hardware circuits.

79. The safety architecture of claim 77, wherein the stimulation output constraints are enforced by hardware and/or hardware-assisted digital circuits.

80. The safety architecture of claim 77, wherein the stimulation output constraints comprise at least one of: a maximum stimulation current, a maximum pulse width, a maximum pulse repetition rate, a maximum burst duration, or a maximum stimulation duty cycle.

81. The safety architecture of claim 74, wherein the isolation barrier (720) defines a low-voltage domain (391) comprising the sensing and control subsystem (200, 300, 500) and an isolated stimulation domain (399) comprising the stimulation subsystem (410, 700), and wherein the stimulation domain (399) operates on an isolated supply and an isolated reference potential.

82-87. (canceled)

88. A platform architecture for intraoral sensing and intervention comprising:
an intraoral interface structure (110, 111, 112) configured to engage teeth;
a sensing subsystem (200);
an event detection subsystem (300) configured to detect events based on a combined evaluation of occlusal force and inertial motion parameters sensed by the sensing subsystem within a predefined temporal window;
an intervention subsystem (400) comprising at least two feedback modalities comprising mechanical feedback and neuromodulation; and
a safety subsystem (800) configured to enforce hardware-defined operational limits independent of software execution.

89. (canceled)

90. The platform architecture of claim 88, wherein the sensing subsystem (200) comprises at least one force sensor and at least one inertial sensor.

91. (canceled)
